(19) United States
(12) Patent Application Publication
(10) Pub. No.: US 2026/0325430 A1
(43) Pub. Date: Oct. 22, 2026

(54) COSMETIC HAIR CARE COMPOSITION COMPRISING AT LEAST ONE PARTICULAR AMINO SILICONE AND AT LEAST ONE NON-SILICONE FATTY SUBSTANCE, AND COSMETIC HAIR TREATMENT PROCESS

(71) Applicants: L'Oreal, Paris (FR); Momentive Performance Materials Inc., Niskayuna, NY (US)

(72) Inventors: Sophie Bourel, SAINT OUEN (FR); Adrien Benazzouz, SAINT OUEN (FR); Anne Dussaud, Tarrytown, NY (US)

(73) Assignees: L'Oreal, Paris (FR); Momentive Performance Materials Inc., Niskayuna, NY (US)

(21) Appl. No.: 19/116,638

(22) PCT Filed: Sep. 29, 2023
(86) PCT No.: PCT/EP2023/077059

§ 371 Date: Mar. 28, 2025

(30) Foreign Application Priority Data
Sep. 30, 2022 (FR) 2209999

(51) Int. Cl. A61K 8/898; A61Q 5/12; C08G 77/26

(52) U.S. Cl. CPC A61K 8/898; A61Q 5/12; C08G 77/26

(57) ABSTRACT

The present invention relates to a cosmetic hair care composition comprising at least one particular amino silicone and at least one non-silicone fatty substance.

The present invention relates to a cosmetic hair care composition comprising at least one particular amino silicone and at least one non-silicone fatty substance. The present invention also relates to a cosmetic hair treatment process, and more particularly a process for cleansing and/or conditioning the hair, comprising the application of a composition according to the invention.

The hair is sensitive to many types of attack and can be embrittled and damaged to various extents by the external environment, in particular by the action of atmospheric agents such as light, atmospheric pollution and bad weather, and also by mechanical or chemical treatments, such as brushing, combing, dyeing, bleaching, permanent-waving and/or relaxing, or even repeated washing.

The properties of damaged hair are then degraded and various manifestations of the damage can be seen, such as difficulties in disentangling and in styling the head of hair, a lack of softness of the hair, hair which can over time become dry, brittle or dull, in particular at fragile spots, and more particularly at the ends.

To overcome these drawbacks, and in particular to repair damaged hair, it is common practice to make use of cosmetic hair compositions for conditioning the hair by giving it satisfactory cosmetic properties. Current compositions generally aim to reduce surface defects of the hair in particular by depositing compounds with a conditioning affinity, such as silicones, and more particularly amino silicones. The effects obtained are generally based on the use of high contents of silicones, which however may have the drawback of a weighing-down effect, or loss of lightness, of the head of hair.

In addition, although there are numerous care formulas for hair repair, they do not generally give the hair entirely satisfactory and long-lasting cosmetic properties, most particularly when the hair is very embrittled, sensitized or damaged. Thus, it has been observed that, for the most damaged hair, the level of care may prove insufficient; in particular, the smooth nature (visual and to the touch) of the head of hair, and its softness, could be further improved.

Moreover, these effects can only generally be discerned at the time of application and for a few hours thereafter.

Finally, the use of high silicone contents may be viewed unfavourably by consumers.

The aim of the invention is therefore to propose a composition for cleansing and/or conditioning the hair which solves the above-mentioned problems, and in particular will provide a high level of care and conditioning to the hair, in particular to the most damaged hair, these effects being discernible even at low contents of amino silicone.

Another aim of the present invention is to propose compositions which are effective in terms of the cosmetic properties given to the hair, both at the time of application and also in a manner that lasts over time.

These aims and others are achieved by the present invention, a subject of which is therefore a cosmetic hair care composition, comprising:
at least one amino silicone of formula (I) as defined below; and
at least one non-silicone fatty substance.

It has been observed that the composition according to the invention is easy to rinse off, making it possible to avoid using large quantities of water to eliminate it, where appropriate, for example when the composition is in the form of a shampoo or conditioner to be rinsed off.

After application, it gives a good level of care, in particular a smooth nature to the touch and visually, softness, a uniform coating from the root to the end, and therefore repair of damaged ends, and also ultimately a clean and natural feel to the hair, without weighing it down. The care and repair benefits are highly pronounced, while maintaining added value in terms of lightness and cleanliness.

Better curl definition when the composition is applied to curly hair, a hair manageability and control effect, and good control of volume have also been observed.

These care, lightness and clean feel properties are significant immediately after application and remain discernible for at least 24 hours, or until the next shampoo wash. They may even persist after a shampoo wash.

In addition, these cosmetic performance qualities, in particular of disentangling, softness, smoothness and coating, may advantageously be obtained with a low silicone content, in particular a lower content than the content customarily used.

In the present description, and unless otherwise indicated:
the term “molecular weight” is equivalent to the term “molecular mass”;
the expression “at least one” is equivalent to the expression “one or more” and can be replaced therewith;
the expression “between” is equivalent to the expression “ranging from” and can be replaced therewith, and implies that the limits are included.

In the context of the invention, “cosmetic hair care composition” means a cosmetic composition for washing (or cleansing) the hair and/or for conditioning the hair.

In the context of the present invention, the cosmetic hair care composition is advantageously a pre-shampoo, a shampoo, a conditioner, a hair mask, a hair serum, it being possible for these compositions to be rinsed off or left on after application.

### Amino Silicone

The composition according to the invention comprises at least one amino silicone of formula (I)

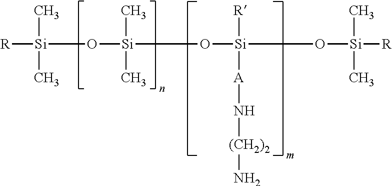

in which:
R, which is identical or different, represents a methyl group (CH₃), a hydroxyl radical (OH), or an alkoxy group comprising 1 to 4 carbon atoms (OR1, where R1═C1-C4 alkyl);
R′ represents a methyl group (CH₃) or a hydroxyl radical (OH);
A is a linear or branched divalent alkylene group comprising 3 or 4 carbon atoms; the amino silicone having an amine number ranging from 0.1 to 0.29 meq/g and a weight-average molecular mass (Mw) ranging from 10 000 to 100 000.

Preferably, R, which is identical or different, represents a methyl group and/or a hydroxyl radical. Better still, the two radicals R are identical and represent a methyl group or a hydroxyl radical, even better still a methyl radical.

Preferably, R′ represents a methyl group.

Preferably, A is a linear divalent alkylene group comprising 3 or 4 carbon atoms; better still, a linear group comprising 3 carbon atoms (—CH₂CH₂CH₂—).

In formula (I), the values of n and m are chosen such that the amino silicone has an amine number ranging from 0.1 to 0.29 meq/g and a weight-average molecular mass (Mw) ranging from 10 000 to 100 000.

Preferably, the amino silicone of formula (I) has an amine number ranging from 0.1 to 0.25 meq/g, better still ranging from 0.1 to 0.19 meq/g, and even better still ranging from 0.1 to 0.16 meq/g.

Preferably, the amino silicone of formula (I) has a weight-average molecular mass (Mw) ranging from 20 000 to 70 000, better still from 25 000 to 50 000, even better still from 35 000 to 45 000.

Those skilled in the art, on the basis of their general knowledge, will know how to determine the values of n and m in order to obtain a silicone having the desired amine number and weight-average molecular mass.

Preferably, in formula (I), m is between 1.4 and 13, better still between 1.4 and 9, even better still between 1.5 and 5, and the n/m ratio is between 45 and 330, better still between 100 and 300.

The value of n can thus readily be determined on the basis of all this data; in particular, n can be between 400 and 600.

According to a particularly preferred embodiment, the composition according to the invention comprises one or more amino silicones of formula (I):

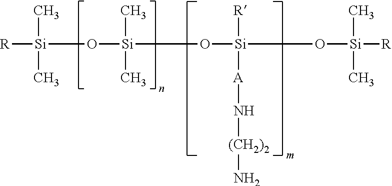

in which:
R, which are identical, and R′, represent a methyl group,
A is a linear or branched divalent alkylene group comprising 3 or 4 carbon atoms; preferably a linear divalent alkylene group comprising 3 carbon atoms (—CH₂CH₂CH₂—), the amino silicone has an amine number ranging from 0.1 to 0.19 meq/g and a weight-average molecular weight (Mw) ranging from 25 000 to 50 000.

Preferably, in this embodiment, m is between 1.4 and 13, the n/m ratio is between 45 and 300, and n can be between 400 and 600.

The amino silicone of formula (I) preferably has a dynamic viscosity, measured at 25° C., 1 atm, ranging from 2 to 8 Pa·s (2000-8000 cps), better still from 3 to 6 Pa·s (3000-6000 cps), even better still from 3 to 5 Pa·s.

Preferably, the composition according to the invention comprises the amino silicone(s) of formula (I) in a total content which can range from 0.001% to 10% by weight, in particular from 0.002% to 5% by weight, better still from 0.005% to 3% by weight, even better still from 0.01% to 2.5% by weight, preferentially from 0.02% to 2% by weight, better still from 0.05% to 1.5% by weight and even better still from 0.1% to 1.2% by weight, relative to the total weight of the composition.

The amino silicone of formula (I) can be prepared by any means conventionally employed in the silicone industry.

### Non-Silicone Fatty Substances

The composition according to the invention comprises at least one non-silicone fatty substance, which may be chosen from solid fatty substances, liquid fatty substances, and mixtures thereof.

The term “non-silicone fatty substance” means a fatty substance not containing any Si—O bonds.

The term “solid fatty substance” means a fatty substance having a melting point of greater than 25° C., preferably greater than or equal to 28° C., preferentially greater than or equal to 30° C., at atmospheric pressure (1.013×10⁵ Pa).

Advantageously, the solid fatty substances that may be used in the present invention are neither (poly)oxyalkylenated nor (poly)glycerolated.

The solid fatty substances may be chosen from solid fatty acids, solid fatty alcohols, solid esters of fatty acids and/or of fatty alcohols, waxes and ceramides, and mixtures thereof.

“Fatty acid” means a long-chain carboxylic acid comprising from 6 to 40 carbon atoms, preferably from 8 to 30 carbon atoms. The solid fatty acids according to the invention preferentially comprise from 10 to 30 carbon atoms and better still from 14 to 22 carbon atoms. These fatty acids are neither oxyalkylenated nor glycerolated. The solid fatty acids that may be used in the present invention are particularly chosen from myristic acid, cetylic acid, stearylic acid, palmitic acid, stearic acid, lauric acid, behenic acid, and mixtures thereof. Said fatty acids are other than the (poly)hydroxylated carboxylic acids comprising from 2 to 8 carbon atoms described previously.

“Fatty alcohol” means a long-chain aliphatic alcohol comprising from 6 to 40 carbon atoms, preferably from 8 to 30 carbon atoms, and comprising at least one hydroxyl group OH. These fatty alcohols are neither oxyalkylenated nor glycerolated. The solid fatty alcohols may be saturated or unsaturated, and linear or branched, and include from 8 to 40 carbon atoms, preferably from 10 to 30 carbon atoms, better still from 12 to 30 carbon atoms. Preferably, the solid fatty alcohols have the structure R—OH with R denoting a linear alkyl group, optionally substituted with one or more hydroxyl groups, comprising from 8 to 40, preferentially from 10 to 30 carbon atoms, better still from 12 to 30, or even from 12 to 24 atoms and even better still from 14 to 22 carbon atoms. The solid fatty alcohols that may be used are preferably chosen from saturated, and linear or branched, preferably linear and saturated, (mono)alcohols including from 8 to 40 carbon atoms, better still from 10 to 30, or even from 12 to 24 atoms and even better still from 14 to 22 carbon atoms.

The solid fatty alcohols that can be used may be chosen, alone or as a mixture, from:
myristyl alcohol (or 1-tetradecanol);
cetyl alcohol (or 1-hexadecanol);
stearyl alcohol (or 1-octadecanol);
arachidyl alcohol (or 1-eicosanol);
behenyl alcohol (or 1-docosanol);
lignoceryl alcohol (or 1-tetracosanol);
ceryl alcohol (or 1-hexacosanol);
montanyl alcohol (or 1-octacosanol);
myricyl alcohol (or 1-triacontanol).

Preferentially, the solid fatty alcohol is chosen from cetyl alcohol, stearyl alcohol, behenyl alcohol, myristyl alcohol, arachidyl alcohol, and mixtures thereof, such as cetylstearyl or cetearyl alcohol. Particularly preferably, the solid fatty alcohol is chosen from cetyl alcohol, stearyl alcohol or mixtures thereof, such as cetylstearyl alcohol; better still, the solid fatty alcohol is cetylstearyl alcohol.

The solid esters of a fatty acid and/or of a fatty alcohol that may be used are preferably chosen from esters derived from a C₉-C₂₆ carboxylic fatty acid and/or from a C₉-C₂₆ fatty alcohol.

Preferably, these solid fatty esters are esters of a linear or branched, saturated carboxylic acid including at least 10 carbon atoms, preferably from 10 to 30 carbon atoms and more particularly from 12 to 24 carbon atoms, and of a linear or branched, saturated monoalcohol including at least 10 carbon atoms, preferably from 10 to 30 carbon atoms and more particularly from 12 to 24 carbon atoms. The saturated carboxylic acids may optionally be hydroxylated, and are preferably monocarboxylic acids.

Esters of C₄-C₂₂ dicarboxylic or tricarboxylic acids and of C₁-C₂₂ alcohols and esters of mono-, di- or tricarboxylic acids and of C₂-C₂₆ di-, tri-, tetra- or pentahydroxy alcohols may also be used.

Mention may particularly be made of octyldodecyl behenate, isocetyl behenate, cetyl lactate, stearyl octanoate, octyl octanoate, cetyl octanoate, decyl oleate, hexyl stearate, octyl stearate, myristyl stearate, cetyl stearate, stearyl stearate, octyl pelargonate, cetyl myristate, myristyl myristate, stearyl myristate, diethyl sebacate, diisopropyl sebacate, diisopropyl adipate, di-n-propyl adipate, dioctyl adipate, dioctyl maleate, octyl palmitate, myristyl palmitate, cetyl palmitate, stearyl palmitate, and mixtures thereof.

Preferably, the solid esters of a fatty acid and/or of a fatty alcohol are chosen from C₉-C₂₆ alkyl palmitates, particularly myristyl palmitate, cetyl palmitate and stearyl palmitate; C₉-C₂₆ alkyl myristates, such as cetyl myristate, stearyl myristate and myristyl myristate; C₉-C₂₆ alkyl stearates, particularly myristyl stearate, cetyl stearate and stearyl stearate; and mixtures thereof.

Particularly preferably, the solid esters of a fatty acid and/or of a fatty alcohol are chosen from myristyl stearate, myristyl palmitate and mixtures thereof.

For the purposes of the present invention, a wax is a lipophilic compound, which is solid at 25′ C and atmospheric pressure, with a reversible solid/liquid change of state, having a melting point of greater than approximately 40° C. and which may be up to 200° C., and having anisotropic crystal organization in the solid state. In general, the size of the wax crystals is such that the crystals diffract and/or scatter light, giving the composition that comprises them a relatively opaque cloudy appearance. By bringing the wax to its melting point, it is possible to make it miscible with oils and to form a microscopically homogeneous mixture, but on returning the temperature of the mixture to ambient temperature, recrystallization of the wax, which is microscopically and macroscopically detectable (opalescence), is obtained.

In particular, the waxes that are suitable for use in the invention may be chosen from waxes of animal, plant or mineral origin, non-silicone synthetic waxes, and mixtures thereof.

Mention may particularly be made of hydrocarbon-based waxes, for instance beeswax or modified beeswaxes (cera bellina), lanolin wax and lanolin derivatives, spermaceti; cork fibre or sugarcane waxes, olive tree wax, rice bran wax, carnauba wax, candelilla wax, ouricury wax, esparto grass wax, berry wax, shellac wax, Japan wax and sumac wax, absolute waxes of flowers; montan wax, orange wax, lemon wax, microcrystalline waxes, paraffins, petroleum jelly, lignite and ozokerite; polyethylene waxes, the waxes obtained by Fischer-Tropsch synthesis and waxy copolymers, and also esters thereof. Mention may also be made of C2 to C60 microcrystalline waxes, such as Microwax HW.

Mention may also be made of the MW 500 polyethylene wax sold under the reference Permalen 50-L Polyethylene.

Mention may also be made of waxes obtained by catalytic hydrogenation of animal or plant oils having linear or branched C₈ to C₃₂ fatty chains.

Among these waxes, mention may particularly be made of isomerized jojoba oil such as trans-isomerized partially hydrogenated jojoba oil, particularly the product manufactured or sold by the company Desert Whale under the commercial reference Iso-Jojoba-50®, hydrogenated sunflower oil, hydrogenated castor oil, hydrogenated coconut kernel oil, hydrogenated lanolin oil and bis(1,1,1-trimethylolpropane) tetrastearate, particularly the product sold under the name Hest 2T-4S® by the company Heterene. The waxes obtained by hydrogenation of castor oil esterified with cetyl alcohol, such as those sold under the names Phytowax Castor 16L64® and 22L73® by the company Sophim, may also be used.

A wax that may also be used is a C20 to C40 alkyl (hydroxystearyloxy)stearate (the alkyl group comprising from 20 to 40 carbon atoms), alone or as a mixture. Such a wax is particularly sold under the names “Kester Wax K 82 P®”, “Hydroxypolyester K 82 P®” and “Kester Wax K 80 P®” by Koster Keunen.

It is also possible to use microwaxes in the compositions of the invention; mention may particularly be made of carnauba microwaxes, such as the product sold under the name MicroCare 350® by the company Micro Powders, synthetic-wax microwaxes, such as the product sold under the name MicroEase 114S® by the company Micro Powders, microwaxes constituted of a mixture of carnauba wax and polyethylene wax, such as the products sold under the names Micro Care 300® and 310® by the company Micro Powders, microwaxes constituted of a mixture of carnauba wax and of synthetic wax, such as the product sold under the name Micro Care 325® by the company Micro Powders, polyethylene microwaxes, such as the products sold under the names Micropoly 200®, 220®, 220L® and 250S® by the company Micro Powders, and polytetrafluoroethylene microwaxes, such as the products sold under the names Microslip 519® and 519 L® by the company Micro Powders.

The waxes are preferably chosen from mineral waxes, for instance paraffin, petroleum jelly, lignite or ozokerite wax; plant waxes, for instance cocoa butter, shea butter or cork fibre or sugar cane waxes, olive tree wax, rice bran wax, hydrogenated jojoba wax, ouricury wax, carnauba wax, candelilla wax, esparto grass wax, or absolute waxes of flowers, such as the essential wax of blackcurrant blossom sold by the company Bertin (France); waxes of animal origin, for instance beeswaxes or modified beeswaxes (cera bellina), spermaceti, lanolin wax and lanolin derivatives; microcrystalline waxes; and mixtures thereof.

Ceramides, or ceramide analogues, such as glycoceramides, that may be used in the compositions according to the invention, are known; mention may in particular be made of ceramides of classes I, II, III and V according to the Dawning classification. The ceramides or analogues thereof that may be used preferably correspond to the following formula:

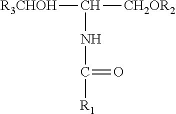

in which:
R₁ denotes a linear or branched, saturated or unsaturated alkyl group, derived from C₁₄-C₃₀ fatty acids, it being possible for this group to be substituted with a hydroxyl group in the alpha position, or a hydroxyl group in the omega position esterified with a saturated or unsaturated C₁₆-C₃₀ fatty acid;
R₂ denotes a hydrogen atom, a (glycosyl)ₙ group, a (galactosyl)ₘ, group or a sulfogalactosyl group, in which n is an integer ranging from 1 to 4 and m is an integer ranging from 1 to 8;
R₃ denotes a C₁₅-C₂₆ hydrocarbon-based group, which is saturated or unsaturated in the alpha position, it being possible for this group to be substituted with one or more C₁-C₁₄ alkyl groups;
it being understood that, in the case of natural ceramides or glycoceramides, R₃ may also denote a C₁₅-C₂₆ alpha-hydroxyalkyl group, the hydroxyl group optionally being esterified with a C₁₆-C₃₀ alpha-hydroxy acid.

Preferentially, use is made of ceramides for which R₁ denotes a saturated or unsaturated alkyl group derived from C₁₄-C₃₀ fatty acids; R₂ denotes a galactosyl or sulfogalactosyl group; and R₃ denotes a —CH═CH—(CH₂)₁₂—CH₃ group.

The ceramides that are more particularly preferred are the compounds for which R₁ denotes a saturated or unsaturated alkyl derived from C₁₆-C₂₂ fatty acids; R₂ denotes a hydrogen atom and R₃ denotes a saturated or unsaturated linear C₁₅ group.

Use may also be made of the compounds for which R₁ denotes a saturated or unsaturated alkyl radical derived from C₁₂-C₂₂ fatty acids; R₂ denotes a galactosyl or sulfogalactosyl radical; and R₃ denotes a saturated or unsaturated C₁₂-C₂₂ hydrocarbon-based radical and preferably a —CH═CH—(CH₂)₁₂—CH₃ group.

As compounds that are particularly preferred, mention may also be made of 2-N-linoleoylaminooctadecane-1,3-diol; 2-N-oleoylaminooctadecane-1,3-diol; 2-N-palmitoylaminooctadecane-1,3-diol; 2-N-stearoylaminooctadecane-1,3-diol; 2-N-behenoylaminooctadecane-1,3-diol; 2-N-[2-hydroxypalmitoyllaminooctadecane-1,3-diol; 2-N-stearoylaminooctadecane-1,3,4-triol and in particular N-stearoylphytosphingosine, 2-N-palmitoylaminohexadecane-1,3-diol, N-linoleoyldihydrosphingosine, Noleoyldihydrosphingosine, N-palmitoyldihydrosphingosine, N-stearoyldihydrosphingosine, and N-behenoyldihydrosphingosine, N-docosanoyl-N-methyl-D-glucamine, cetylic acid N-(2-hydroxyethyl)-N-(3-cetyloxy-2-hydroxypropyl)amide and bis(N-hydroxyethyl-N-cetyl)malonamide; and mixtures thereof. Use will preferably be made of Noleoyldihydrosphingosine.

As liquid fatty substances that may be used, mention may be made of liquid hydrocarbons, liquid fatty alcohols, liquid esters of fatty acids and/or fatty alcohols other than the triglycerides, oils of triglyceride type of plant or synthetic origin, mineral oils and mixtures thereof.

The liquid fatty substances have a melting point of less than or equal to 25° C., preferably of less than or equal to 20° C., at atmospheric pressure (1.013×10⁵ Pa). Advantageously, the liquid fatty substances are not (poly)oxyalkylenated.

It is recalled that the fatty alcohols, esters and acids more particularly have at least one saturated or unsaturated, linear or branched hydrocarbon-based group comprising from 6 to 40 and better still from 8 to 30 carbon atoms, which is optionally substituted, in particular with one or more hydroxyl groups (in particular 1 to 4). If they are unsaturated, these compounds may comprise one to three conjugated or unconjugated carbon-carbon double bonds.

The liquid hydrocarbons may be C₆ to C₁₈ liquid hydrocarbons and be linear, branched or optionally cyclic; they are preferably chosen from C₈-C₁₆, particularly C₁₀-C₁₄, alkanes. Examples that may be mentioned include hexane, cyclohexane, undecane, dodecane, isododecane, tridecane or isoparaffins, such as isohexadecane or isodecane, and mixtures thereof.

The liquid hydrocarbons may also be chosen from those comprising more than 16 carbon atoms, which may be linear or branched, of mineral or synthetic origin; mention may be made of liquid paraffins or liquid petroleum jelly, polydecenes, hydrogenated polyisobutene, such as Parleam®, and mixtures thereof.

The triglyceride oils of plant or synthetic origin may be chosen from liquid fatty acid triglycerides including from 6 to 30 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by the company Stéarinerie Dubois or those sold under the names Miglyol® 810, 812 and 818 by the company Dynamit Nobel, jojoba oil and shea butter oil, and mixtures thereof. The liquid fatty alcohols may be chosen from linear or branched, saturated or unsaturated alcohols, preferably unsaturated or branched alcohols, including from 6 to 40 carbon atoms and preferably from 8 to 30 carbon atoms. Examples that may be mentioned include octyldodecanol, 2-butyloctanol, 2-hexyldecanol, 2-undecylpentadecanol, isostearyl alcohol, oleyl alcohol, linolenyl alcohol, ricinoleyl alcohol, undecylenyl alcohol and linoleyl alcohol, and mixtures thereof.

As regards the liquid esters of fatty acids and/or fatty alcohols other than the triglycerides mentioned above, mention may particularly be made of esters of saturated or unsaturated, linear C₁ to C₂₆ or branched C₃ to C₂₆, aliphatic monoacids or polyacids and of saturated or unsaturated, linear C₁ to C₂₆ or branched C₃ to C₂₆, aliphatic monoalcohols or polyalcohols, the total carbon number of the esters being greater than or equal to 6, more advantageously greater than or equal to 10.

Preferably, for the esters of monoalcohols, at least one of the alcohol or the acid, from which the esters of the invention are derived, is branched.

Among the monoesters, mention may be made of dihydroabietyl behenate; octyldodecyl behenate; isocetyl behenate; isostearyl lactate; lauryl lactate; linoleyl lactate; oleyl lactate; isostearyl octanoate; isocetyl octanoate; octyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; octyl isononanoate; 2-ethylhexyl isononate; octyldodecyl erucate; oleyl erucate; ethyl and isopropyl palmitates such as 2-ethylhexyl palmitate, 2-octyldecyl palmitate; alkyl myristates such as isopropyl myristate, 2-octyldodecyl myristate, isobutyl stearate; 2-hexyldecyl laurate, and mixtures thereof.

Preferably, among the monoesters of monoacids and of monoalcohols, use will be made of ethyl palmitate or isopropyl palmitate, alkyl myristates, such as isopropyl or ethyl myristate, isocetyl stearate, 2-ethylhexyl isononanoate, isodecyl neopentanoate, isostearyl neopentanoate and mixtures thereof.

Still within the context of this variant, esters of C₄ to C₂₂ dicarboxylic or tricarboxylic acids and of C₁ to C₂₂ alcohols and esters of mono-, di- or tricarboxylic acids and of C₂ to C₂₆ di-, tri-, tetra- or pentahydroxy alcohols may also be used.

Mention may particularly be made of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates, and mixtures thereof.

The composition may also comprise, as fatty ester, sugar esters and diesters of C₆ to C₃₀, preferably C₁₂ to C₂₂, fatty acids. It is recalled that “sugar” means oxygen-containing hydrocarbon-based compounds bearing several alcohol functions, with or without aldehyde or ketone functions, and which comprise at least 4 carbon atoms. These sugars may be monosaccharides, oligosaccharides or polysaccharides.

Examples of suitable sugars that may be mentioned include sucrose (or saccharose), glucose, galactose, ribose, fucose, maltose, fructose, mannose, arabinose, xylose and lactose, and derivatives thereof, particularly alkyl derivatives, such as methyl derivatives, for instance methylglucose.

The sugar esters of fatty acids may be chosen particularly from the group comprising the esters or mixtures of esters of sugars described previously and of linear or branched, saturated or unsaturated C₆ to C₃₀ and preferably C₁₂ to C₂₂ fatty acids. If they are unsaturated, these compounds may comprise one to three conjugated or unconjugated carbon-carbon double bonds.

The esters according to this variant may also be chosen from mono-, di-, tri- and tetraesters, polyesters, and mixtures thereof.

These esters may be, for example, oleates, laurates, palmitates, myristates, behenates, cocoates, stearates, linoleates, linolenates, caprates, arachidonates or mixtures thereof, particularly such as the mixed oleo-palmitate, oleo-stearate and palmito-stearate esters.

More particularly, use is made of monoesters and diesters and particularly sucrose, glucose or methylglucose mono- or di-oleates, stearates, behenates, oleopalmitates, linoleates, linolenates and oleostearates, and mixtures thereof.

Mention may be made, by way of example, of the product sold under the name Glucate® DO by the company Amerchol, which is a methylglucose dioleate.

Preferably, use will be made of a liquid ester of a monoacid and of a monoalcohol.

Preferably, the non-silicone fatty substances are chosen from triglyceride oils of plant or synthetic origin, liquid esters of a fatty acid and/or a fatty alcohol other than triglycerides, liquid C₆-C₁₈ hydrocarbons, solid fatty alcohols, liquid fatty alcohols, solid esters of fatty acids and/or of fatty alcohols, and mixtures thereof.

Preferably, when they are present, the composition according to the invention may comprise the non-silicone fatty substance(s) in a total amount ranging from 0.1% to 20% by weight, better still from 1% to 18% by weight, preferentially from 2% to 15% by weight, even better still from 3% to 13% by weight, relative to the total weight of the composition.

### Additional Ingredients

The composition according to the invention may be any form that can be envisaged by those skilled in the art, and may comprise the additional ingredients customarily employed in this type of composition.

These hair care compositions may for example be shampoos, pre-shampoos (to be applied before a shampoo wash), conditioners, masks or serums, it being possible for these compositions to be rinsed off or left on after application.

In a known manner, they may be in the form of gels, hair lotions and relatively thick care creams.

They may therefore comprise, in a known manner, one or more surfactants, one or more non-silicone fatty substances, one or more additional silicones, one or more polymers, one or more colouring agents, one or more thickeners, one or more polyols, and also a mixture of these various ingredients.

In particular, when the composition according to the invention is a shampoo, it may advantageously comprise one or more anionic surfactants, and/or one or more amphoteric surfactants, and/or one or more nonionic surfactants, and/or one or more polymers, particularly cationic polymers.

In particular, when the composition of the invention is a pre-shampoo, a conditioner or a mask, it may advantageously comprise one or more cationic surfactants and/or one or more non-silicone fatty substances.

Regardless of its intended use, the composition according to the invention may advantageously comprise one or more additional silicones, one or more thickeners, and/or one or more polyols.

### Pigments

The composition according to the invention may thus comprise at least one pigment. Preferably, the composition according to the invention comprises at least one pigment. “Pigment” means all pigments that give colour to keratin materials. Their solubility in water at 25° C. and at atmospheric pressure (760 mmHg) is less than 0.05% by weight, and preferably less than 0.01%.

The pigments that may be used are particularly chosen from the organic and/or mineral pigments known in the art, particularly those described in Kirk-Othmer's Encyclopedia of Chemical Technology and in Ullmann's Encyclopedia of Industrial Chemistry.

They may be natural, of natural origin, or non-natural.

These pigments may be in pigment powder or paste form. They may be coated or uncoated.

The pigments may be chosen, for example, from mineral pigments, organic pigments, lakes, special effect pigments such as nacres or glitter flakes, lamellar pigments, and mixtures thereof.

The pigment may be a mineral pigment. “Mineral pigment” means any pigment that satisfies the definition in Ullmann's encyclopedia in the chapter on inorganic pigments. Among the mineral pigments that are useful in the present invention, mention may be made of iron oxides, chromium oxides, manganese violet, ultramarine blue, chromium hydrate, ferric blue and titanium oxide.

The pigment may be an organic pigment. “Organic pigment” means any pigment that satisfies the definition in Ullmann's Encyclopedia in the chapter on organic pigments.

The organic pigment may particularly be chosen from nitroso, nitro, azo, xanthene, pyrene, quinoline, anthraquinone, triphenylmethane, fluorane, phthalocyanine, metalcomplex, isoindolinone, isoindoline, quinacridone, perinone, perylene, diketopyrrolopyrrole, indigo, thioindigo, dioxazine, triphenylmethane and quinophthalone compounds.

In particular, the white or coloured organic pigments may be chosen from carmine, carbon black, aniline black, azo yellow, quinacridone, phthalocyanine blue, the blue pigments codified in the Colour Index under the references CI 42090, 69800, 69825, 74100, 74160, the yellow pigments codified in the Colour Index under the references CI 11680, 11710, 19140, 20040, 21100, 21108, 47000, 47005, the green pigments codified in the Colour Index under the references CI 61565, 61570, 74260, the orange pigments codified in the Colour Index under the references CI 11725, 45370, 71105, the red pigments codified in the Colour Index under the references CI 12085, 12120, 12370, 12420, 12490, 14700, 15525, 15580, 15620, 15630, 15800, 15850, 15865, 15880, 26100, 45380, 45410, 58000, 73360, 73915, 75470, the pigments obtained by oxidative polymerization of indole or phenol derivatives as described in patent FR 2 679 771.

Examples that may also be mentioned include pigment pastes of organic pigments, such as the products sold by the company Hoechst under the names:
Cosmenyl Yellow 10G: Yellow 3 pigment (CI 11710);
Cosmenyl Yellow G: Yellow 1 pigment (CI 11680);
Cosmenyl Orange GR: Orange 43 pigment (CI 71105);
Cosmenyl Red R: Red 4 pigment (CI 12085);
Cosmenyl Carmine FB: Red 5 pigment (CI 12490);
Cosmenyl Violet RL: Violet 23 pigment (CI 51319);
Cosmenyl Blue A2R: Blue 15.1 pigment (CI 74160);
Cosmenyl Green GG: Green 7 pigment (CI 74260);
Cosmenyl Black R: Black 7 pigment (CI 77266).

The pigments in accordance with the invention may also be in the form of composite pigments, as described in patent EP 1184 426. These composite pigments may particularly be composed of particles including an inorganic core, at least one binder for attaching the organic pigments to the core, and at least one organic pigment which at least partially covers the core.

The organic pigment may also be a lake. “Lake” means dyes adsorbed onto insoluble particles, the assembly thus obtained remaining insoluble during use.

The inorganic substrates onto which the dyes are adsorbed are, for example, alumina, silica, calcium sodium borosilicate or calcium aluminium borosilicate and aluminium.

Among the dyes, mention may be made of carminic acid. Mention may also be made of the dyes known under the following names: D & C Red 21 (CI 45 380), D & C Orange 5 (CI 45 370), D & C Red 27 (CI 45 410), D & C Orange 10 (CI 45 425), D & C Red 3 (CI 45 430), D & C Red 4 (CI 15 510), D & C Red 33 (CI 17 200), D & C Yellow 5 (CI 19 140), D & C Yellow 6 (CI 15 985), D & C Green (CI 61 570), D & C Yellow 1 O (CI 77 002), D & C Green 3 (CI 42 053), D & C Blue 1 (CI 42 090).

An example of a lake that may be mentioned is the product known under the following name: D&C Red 7 (CI 15 850:1).

The pigment may also be a special effect pigment. “Special effect pigments” means pigments that generally create a coloured appearance (characterized by a certain shade, a certain intensity and a certain level of luminance) that is non-uniform and that changes based on the conditions of observation (light, temperature, angles of observation, etc.). They are thus in contrast to coloured pigments, which afford a standard uniform opaque, semi-transparent or transparent hue.

Several types of special effect pigments exist: those with a low refractive index, such as fluorescent or photochromic pigments, and those with a higher refractive index, such as nacres, interference pigments or glitter flakes.

Examples of special effect pigments that may be mentioned include nacreous pigments such as mica covered with titanium or with bismuth oxychloride, coloured nacreous pigments such as mica covered with titanium and with iron oxides, mica covered with iron oxide, mica covered with titanium and particularly with ferric blue or with chromium oxide, mica covered with titanium and with an organic pigment as defined previously, and also nacreous pigments based on bismuth oxychloride. Nacreous pigments that may be mentioned include the nacres Cellini sold by BASF (mica-TiO2-lake), Prestige sold by Eckart (mica-TiO2), Prestige Bronze sold by Eckart (mica-Fe2O3), and Colorona sold by Merck (mica-TiO2-Fe2O3).

Mention may also be made of the gold-coloured nacres sold particularly by the company BASF under the name Brilliant gold 212G (Timica), Gold 222C (Cloisonne), Sparkle gold (Timica), Gold 4504 (Chromalite) and Monarch gold 233X (Cloisonne); the bronze nacres sold particularly by the company Merck under the name Bronze fine (17384) (Colorona) and Bronze (17353) (Colorona) and by the company BASF under the name Super bronze (Cloisonne); the orange nacres sold particularly by the company BASF under the name Orange 363C (Cloisonne) and Orange MCR 101 (Cosmica) and by the company Merck under the name Passion orange (Colorona) and Matte orange (17449) (Microna); the brown nacres sold particularly by the company BASF under the name Nu-antique copper 340XB (Cloisonne) and Brown CL4509 (Chromalite); the nacres with a copper tint sold particularly by the company BASF under the name Copper 340A (Timica); the nacres with a red tint sold particularly by the company Merck under the name Sienna fine (17386) (Colorona); the nacres with a yellow tint sold particularly by the company BASF under the name Yellow (4502) (Chromalite); the red nacres with a gold tint sold particularly by the company BASF under the name Sunstone G012 (Gemtone); the pink nacres sold particularly by the company BASF under the name Tan opale G005 (Gemtone); the black nacres with a gold tint sold particularly by the company BASF under the name Nu antique bronze 240 AB (Timica), the blue nacres sold particularly by the company Merck under the name Matte blue (17433) (Microna), the white nacres with a silvery tint sold particularly by the company Merck under the name Xirona Silver, and the golden-green pink-orange nacres sold particularly by the company Merck under the name Indian summer (Xirona), and mixtures thereof.

Still as examples of nacres, mention may also be made of particles including a borosilicate substrate coated with titanium oxide.

Particles comprising a glass substrate coated with titanium oxide are particularly sold under the name Metashine MC1080RY by the company Toyal.

Finally, examples of nacres that may also be mentioned include polyethylene terephthalate glitter flakes, particularly those sold by the company Meadowbrook Inventions under the name Silver 1P 0.004×0.004 (silver-coloured glitter flakes). It is also possible to envisage multilayer pigments based on synthetic substrates, such as alumina, silica, calcium sodium borosilicate, calcium aluminium borosilicate and aluminium.

The special effect pigments may also be chosen from reflective particles, i.e. particularly from particles whose size, structure, particularly the thickness of the layer(s) of which they are made and their physical and chemical nature, and surface state, allow them to reflect incident light. This reflection may, where appropriate, have an intensity sufficient to create, at the surface of the composition or of the mixture, when it is applied to the support to be made up, highlighted points that are visible to the naked eye, i.e. more luminous points that contrast with their environment, appearing to sparkle.

The reflective particles may be selected so as not to significantly alter the colouring effect generated by the colouring agents with which they are combined, and more particularly so as to optimize this effect in terms of colour result. They may more particularly have a yellow, pink, red, bronze, orangey, brown, gold and/or coppery colour or tint.

These particles may have varied forms and may particularly be in platelet or globular form, in particular in spherical form.

The reflective particles, regardless of their form, may or may not have a multilayer structure and, in the case of a multilayer structure, may have, for example, at least one layer of uniform thickness, particularly of a reflective material.

When the reflective particles do not have a multilayer structure, they may be composed, for example, of metal oxides, particularly titanium or iron oxides obtained synthetically.

When the reflective particles have a multilayer structure, they may include, for example, a natural or synthetic substrate, particularly a synthetic substrate at least partially coated with at least one layer of a reflective material, particularly of at least one metal or metallic material. The substrate may be made of one or more organic and/or inorganic materials.

More particularly, it may be chosen from glasses, ceramics, graphite, metal oxides, aluminas, silicas, silicates, particularly aluminosilicates and borosilicates, and synthetic mica, and mixtures thereof, this list not being limiting.

The reflective material may include a layer of metal or of a metallic material.

Reflective particles are particularly described in JP-A-09188830, JP-A-10158450, JP-A10158541, JP-A-07258460 and JP-A-05017710.

Again as an example of reflective particles including a mineral substrate coated with a layer of metal, mention may also be made of particles including a silver-coated borosilicate substrate.

Particles with a silver-coated glass substrate, in the form of platelets, are sold under the name Microglass Metashine REFSX 2025 PS by the company Toyal. Particles with a glass substrate coated with a nickel/chromium/molybdenum alloy are sold under the names Crystal Star GF 550 and GF 2525 by this same company.

Use may also be made of particles comprising a metal substrate, such as silver, aluminium, iron, chromium, nickel, molybdenum, gold, copper, zinc, tin, magnesium, steel, bronze or titanium, said substrate being coated with at least one layer of at least one metal oxide, such as titanium oxide, aluminium oxide, iron oxide, cerium oxide, chromium oxide, silicon oxides and mixtures thereof.

Examples that may be mentioned include aluminium powder, bronze powder or copper powder coated with SiO₂, sold under the name Visionaire by the company Eckart.

Mention may also be made of interference pigments which are not attached to a substrate, such as liquid crystals (Helicones HC from Wacker) or interference holographic glitter flakes (Geometric Pigments or Spectra f/x from Spectratek). Special effect pigments also comprise fluorescent pigments, whether these are substances that are fluorescent in daylight or that produce an ultraviolet fluorescence, phosphorescent pigments, photochromic pigments, thermochromic pigments and quantum dots, sold, for example, by the company Quantum Dots Corporation.

The variety of pigments that may be used in the present invention makes it possible to obtain a wide range of colours, and also particular optical effects such as metallic effects or interference effects.

The size of the pigment used in the composition according to the present invention is generally between 10 nm and 200 μm, preferably between 20 nm and 80 μm and more preferentially between 30 nm and 50 μm.

The pigments may be dispersed in the composition by means of a dispersant.

The dispersant serves to protect the dispersed particles against agglomeration or flocculation thereof. This dispersant may be a surfactant, an oligomer, a polymer or a mixture of several thereof, bearing one or more functionalities with strong affinity for the surface of the particles to be dispersed. In particular, they may become physically or chemically attached to the surface of the pigments. These dispersants also contain at least one functional group that is compatible with or soluble in the continuous medium.

In particular, esters of 12-hydroxystearic acid in particular and of C8 to C20 fatty acid and of polyols such as glycerol or diglycerol are used, such as poly(12-hydroxystearic acid) stearate with a molecular weight of approximately 750 g/mol, such as the product sold under the name Solsperse 21000 by the company Avecia, polyglyceryl-2 dipolyhydroxystearate (CTFA name) sold under the reference Dehymyls PGPH by the company Henkel, or else polyhydroxystearic acid such as the product sold under the reference Arlacel P100 by the company Uniqema, and mixtures thereof.

As other dispersants that may be used in the compositions of the invention, mention may be made of quaternary ammonium derivatives of polycondensed fatty acids, for instance Solsperse 17 000 sold by the company Avecia, and polydimethylsiloxane/oxypropylene mixtures such as those sold by the company Dow Corning under the references DC2-5185 and DC2-5225 C.

The pigments used in the composition may be surface-treated with an organic agent. Thus, the pigments surface-treated beforehand that are useful in the context of the invention are pigments which have been completely or partially subjected to a surface treatment of chemical, electronic, electrochemical, mechanochemical or mechanical nature with an organic agent, such as those described particularly in Cosmetics and Toiletries, February 1990, Vol. 105, pages 53-64, before being dispersed in the composition in accordance with the invention. These organic agents may be chosen, for example, from waxes, for example carnauba wax and beeswax; fatty acids, fatty alcohols and derivatives thereof, such as stearic acid, hydroxystearic acid, stearyl alcohol, hydroxystearyl alcohol and lauric acid and derivatives thereof; anionic surfactants; lecithins; sodium, potassium, magnesium, iron, titanium, zinc or aluminium salts of fatty acids, for example aluminium stearate or laurate; metal alkoxides; polyethylene; (meth)acrylic polymers, for example polymethyl methacrylates; polymers and copolymers containing acrylate units; alkanolamines; silicone compounds, for example silicones, in particular polydimethylsiloxanes; organofluorine compounds, for example perfluoroalkyl ethers; fluorosilicone compounds.

The surface-treated pigments that are useful in the composition may also have been treated with a mixture of these compounds and/or may have been subjected to several surface treatments.

The surface-treated pigments that are useful in the context of the present invention may be prepared according to surface-treatment techniques that are well known to those skilled in the art, or may be commercially available as is.

Preferably, the surface-treated pigments are covered with an organic layer.

The organic agent with which the pigments are treated may be deposited on the pigments by evaporation of solvent, chemical reaction between the molecules of the surface agent or creation of a covalent bond between the surface agent and the pigments.

The surface treatment may thus be performed, for example, by chemical reaction of a surface agent with the surface of the pigments and creation of a covalent bond between the surface agent and the pigments or the fillers. This method is particularly described in patent U.S. Pat. No. 4,578,266.

An organic agent covalently bonded to the pigments will preferably be used.

The agent for the surface treatment may represent from 0.1% to 50% by weight of the total weight of the surface-treated pigment, preferably from 0.5% to 30% by weight and even more preferentially from 1% to 20% by weight of the total weight of the surface-treated pigment.

Preferably, the surface treatments of the pigments are chosen from the following treatments:
a PEG-silicone treatment, for instance the AQ surface treatment sold by LCW;
a methicone treatment, for instance the SI surface treatment sold by LCW;
a dimethicone treatment, for instance the Covasil 3.05 surface treatment sold by LCW;
a dimethicone/trimethylsiloxysilicate treatment, for instance the Covasil 4.05 surface treatment sold by LCW;
a magnesium myristate treatment, for instance the MM surface treatment sold by LCW;
an aluminium dimyristate treatment, for instance the MI surface treatment sold by Miyoshi;
a perfluoropolymethyl isopropyl ether treatment, for instance the FHC surface treatment sold by LCW;
an isostearyl sebacate treatment, for instance the HS surface treatment sold by Miyoshi;
a perfluoroalkyl phosphate treatment, for instance the PF surface treatment sold by Daito;
an acrylate/dimethicone copolymer and perfluoroalkyl phosphate treatment, for instance the FSA surface treatment sold by Daito;
a polymethylhydrogenosiloxane/perfluoroalkyl phosphate treatment, for instance the FS01 surface treatment sold by Daito;
an acrylate/dimethicone copolymer treatment, for instance the ASC surface treatment sold by Daito;
an isopropyl titanium triisostearate treatment, for instance the ITT surface treatment sold by Daito;
an acrylate copolymer treatment, for instance the APD surface treatment sold by Daito;
a perfluoroalkyl phosphate/isopropyl titanium triisostearate treatment, for instance the PF+ITT surface treatment sold by Daito.

According to a particular embodiment of the invention, the dispersant is present with organic or mineral pigments in submicron-sized particulate form in the dye composition.

The term “submicron-sized” or “submicronic” means pigments having a particle size that has been micronized by a micronization method and having a mean particle size of less than a micrometre (μm), in particular between 0.1 and 0.9 μm, and preferably between 0.2 and 0.6 μm.

According to one embodiment, the dispersant and the pigment(s) are present in a (dispersant:pigment) amount of between 1:4 and 4:1, particularly between 1.5:3.5 and 3.5:1 or better still between 1.75:3 and 3:1.

According to a particular embodiment, the dispersant is suitable for dispersing the pigments and is compatible with a condensation-curable formulation.

The term “compatible” means, for example, that said dispersant is miscible in the oily phase of the composition or of the dispersion containing the pigment(s), and it does not retard or reduce the curing. The dispersant is preferably cationic.

The dispersant(s) may thus have a silicone backbone, such as silicone polyether and dispersants of aminosilicone type other than the alkoxysilanes described previously. Among the suitable dispersants that may be mentioned are:
aminosilicones, i.e. silicones comprising one or more amino groups such as those sold under the names and references: BYK LPX 21879 by BYK, GP-4, GP-6, GP-344, GP-851, GP-965, GP-967 and GP-988-1, sold by Genesee Polymers,
silicone acrylates such as Tego® RC 902, Tego® RC 922, Tego® RC 1041, and Tego® RC 1043, sold by Evonik,
polydimethylsiloxane (PDMS) silicones bearing carboxylic groups such as X-22162 and X-22370 by Shin-Etsu, epoxy silicones such as GP-29, GP-32, GP-502, GP-504, GP-514, GP-607, GP-682, and GP-695 by Genesee Polymers, or Tego® RC 1401, Tego® RC 1403, Tego® RC 1412 by Evonik.

According to a particular embodiment, the dispersant(s) are of amino silicone type other than the alkoxysilanes described previously and are cationic.

Preferably, the pigment(s) is (are) chosen from mineral, mixed mineral-organic, or organic pigments.

Preferably, the pigment(s) according to the invention are organic pigments, preferentially organic pigments surface-treated with an organic agent chosen from silicone compounds.

Preferably, the pigment(s) according to the invention are mineral pigments.

Preferably, the pigment(s) according to the invention are chosen from the group constituted by mixtures of mineral pigments.

Preferably, the pigment(s) according to the invention are chosen from the group constituted by mixtures of organic pigments.

Preferably, the pigment(s) according to the invention are chosen from the group constituted by mixtures of organic and mineral pigments.

Preferably, the pigments are pigments chosen from organic pigments, more preferentially from anthraquinone compounds, in particular alizurol purple monosodium salt (ext. Violet 2, CI 60 730).

The pigment(s) may be present in a total content ranging from 0.001% to 10% by weight, preferably from 0.005% to 5% by weight, preferentially from 0.007% to 2% by weight, relative to the total weight of the composition.

### Anionic Surfactants

The composition according to the invention may further comprise at least one anionic surfactant.

Preferably, the composition according to the invention comprises one or more anionic surfactants.

The anionic surfactants are non-silicone surfactants.

The term “anionic surfactant” means a surfactant including, as ionic or ionizable groups, only anionic groups.

In the present description, a species is termed as being “anionic” when it bears at least one permanent negative charge or when it can be ionized to a negatively charged species, under the conditions of use of the composition of the invention (for example the medium or the pH) and not comprising any cationic charge.

The anionic surfactants may be sulfate, sulfonate and/or carboxylic (or carboxylate) surfactants. Needless to say, a mixture of these surfactants may be used.

It is understood in the present description that:
the carboxylate anionic surfactants comprise at least one carboxylic or carboxylate function (—COOH or —COO⁻) and may optionally also comprise one or more sulfate and/or sulfonate functions;
the sulfonate anionic surfactants comprise at least one sulfonate function (—SO₃H or —SO₃⁻) and may optionally also comprise one or more sulfate functions, but do not comprise any carboxylate functions; and
the sulfate anionic surfactants comprise at least one sulfate function but do not comprise any carboxylate or sulfonate functions.

The carboxylic anionic surfactants that may be used thus include at least one carboxylic or carboxylate function (—COOH or —COO⁻).

They may be chosen from the following compounds: acylglycinates, acyllactylates, acylsarcosinates, acylglutamates; alkyl ether carboxylic acids, alkyl(C6-30 aryl)ether carboxylic acids, alkyl-D-galactosideuronic acids, alkylamido ether carboxylic acids; and also the salts of these compounds; the alkyl and/or acyl groups of these compounds including from 6 to 30 carbon atoms, in particular from 12 to 28, even better still from 14 to 24 or even from 16 to 22 carbon atoms; the aryl group preferably denoting a phenyl or benzyl group; it being possible for these compounds to be polyoxyalkylenated, in particular polyoxyethylenated, and then preferably including from 1 to 50 ethylene oxide units, better still from 2 to 10 ethylene oxide units.

Use may also be made of the C₆-C₂₄ alkyl monoesters of polyglycoside-polycarboxylic acids, such as C₆-C₂₄ alkyl polyglycoside-citrates, C₆-C₂₄ alkyl polyglycoside-tartrates and C₆-C₂₄ alkyl polyglycoside-sulfosuccinates, and salts thereof.

Among the above carboxylic surfactants, mention may be made most particularly of polyoxyalkylenated alkyl(amido) ether carboxylic acids and salts thereof, in particular those including from 2 to 50 alkylene oxide groups, in particular ethylene oxide groups, such as the compounds sold by the company Kao under the Akypo names.

The polyoxyalkylenated alkyl(amido) ether carboxylic acids that may be used are preferably chosen from those of formula (1):

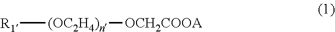

in which:
R_{1′} represents a linear or branched C₆-C₂₄ alkyl or alkenyl radical, a (C₈-C₉)alkylphenyl radical, a R_{2′}CONH—CH₂—CH₂— radical with R_{2′} denoting a linear or branched C₉-C₂₁ alkyl or alkenyl radical; preferably R_{1′} is a C₈-C₂₀, preferably C₈-C₁₈, alkyl radical;
n′ is an integer or decimal number (average value) ranging from 2 to 24 and preferably from 2 to 10,
A denotes H, ammonium, Na, K, Li, Mg or a monoethanolamine or triethanolamine residue.

It is also possible to use mixtures of compounds of formula (1), in particular mixtures of compounds containing different R_{1′} groups.

The polyoxyalkylenated alkyl(amido) ether carboxylic acids that are particularly preferred are those of formula (1) in which:
R_{1′} denotes a linear or branched C₈-C₂₂, in particular C₁₀-C₁₆, or even C₁₂-C₁₄, alkyl radical, or else a (C₈-C₉)alkylphenyl radical;
A denotes a hydrogen or sodium atom, and
n′ ranges from 2 to 20, preferably from 2 to 10.

More preferentially still, use is made of compounds of formula (1) in which R_{1′} denotes a C₁₂-C₁₄ alkyl radical or cocoyl, oleyl, nonylphenyl or octylphenyl radical, A denotes a hydrogen or sodium atom and n′ ranges from 2 to 10.

Among the commercial products that may preferably be used are the products sold by the company Kao under the names:
Akypo® NP 70 (R₁=nonylphenyl, n=7, A=H)
Akypo® NP 40 (R₁=nonylphenyl, n=4, A=H)
Akypo® OP 40 (R₁=octylphenyl, n=4, A=H)
Akypo® OP 80 (R₁=octylphenyl, n=8, A=H)
Akypo® OP 190 (R₁=octylphenyl, n=19, A=H)
Akypo® RLM 38 (R₁═(C₁₂-C₁₄)alkyl, n=4, A=H)
Akypo® RLM 38 NV (R₁═(C₁₂-C₁₄)alkyl, n=4, A=Na)
Akypo® RLM 45 CA (R₁═(C₁₂-C₁₄)alkyl, n=4.5, A=H)
Akypo® RLM 45 NV (R₁═(C₁₂-C₁₄)alkyl, n=4.5, A=Na)
Akypo® RLM 100 (R₁═(C₁₂-C₁₄)alkyl, n=10, A=H)
Akypo® RLM 100 NV (R₁═(C₁₂-C₁₄)alkyl, n=10, A=Na)
Akypo® RLM 130 (R₁═(C₁₂-C₁₄)alkyl, n=13, A=H)
Akypo® RLM 160 NV (R₁═(C₁₂-C₁₄)alkyl, n=16, A=Na),
   or by the company Sandoz under the names:
Sandopan DTC-Acid (R₁═(C₁₃)alkyl, n=6, A=H)
Sandopan DTC (R₁═(C₁₃)alkyl, n=6, A=Na)
Sandopan LS 24 (R₁═(C₁₂-C₁₄)alkyl, n=12, A=Na)
Sandopan JA 36 (R₁═(C₁₃)alkyl, n=18, A=H),
   and more particularly the products sold under the following names:
Akypo® RLM 45 (INCI: Laureth-5 carboxylic acid), Akypo® RLM 100, and Akypo® RLM 38.

Preferentially, the carboxylic anionic surfactants are chosen, alone or as a mixture, from:
C₆-C₂₄ or even C₁₂-C₂₀ acylglutamates, such as stearoylglutamates, and in particular disodium stearoylglutamate;
C₆-C₂₄ or even C₁₂-C₂₀ acylsarcosinates, such as palmitoylsarcosinates, and in particular sodium palmitoylsarcosinate;
C₁₂-C₂₈ or even C₁₄-C₂₄ acyllactylates, such as behenoyllactylates, and in particular sodium behenoyllactylate;
C₆-C₂₄ and in particular C₁₂-C₂₀ acylglycinates;
(C₆-C₂₄)alkyl ether carboxylates, and particularly (C₁₂-C₂₀)alkyl ether carboxylates; in particular those including from 2 to 50 ethylene oxide groups;
polyoxyalkylenated (C₆-C₂₄)alkylamido ether carboxylic acids, in particular those including from 2 to 50 ethylene oxide groups;
   in particular in acid form or in the form of alkali metal or alkaline-earth metal, ammonium or amino alcohol salts.

Polyoxyalkylenated (C₈-C₂₄)alkyl ether carboxylic acids and salts thereof are preferably used.

The sulfonate anionic surfactants that may be used include at least one sulfonate function (—SO₃H or —SO₃⁻). They may be chosen from the following compounds: alkylsulfonates, alkyl ether sulfonates, alkylamidesulfonates, alkylarylsulfonates, α-olefin sulfonates, paraffin sulfonates, alkyl sulfosuccinates, alkyl ether sulfosuccinates, alkylamidesulfosuccinates, alkylsulfoacetates, N-acyltaurates, acylisethionates; alkylsulfolaurates; and also the salts of these compounds;
the alkyl groups of these compounds including from 6 to 30 carbon atoms, particularly from 12 to 28, even better still from 14 to 24 or even from 16 to 22 carbon atoms; the aryl group preferably denoting a phenyl or benzyl group;
it being possible for these compounds to be polyoxyalkylenated, particularly polyoxyethylenated, and then preferably including from 1 to 50 ethylene oxide units and better still from 2 to 10 ethylene oxide units.

Preferentially, the sulfonate anionic surfactants are chosen, alone or as a mixture, from:
C₆-C₂₄ and in particular C₁₂-C₂₀ olefin sulfonates;
C₆-C₂₄ and in particular C₁₂-C₂₀ alkylsulfosuccinates, in particular laurylsulfosuccinates;
C₆-C₂₄ and in particular C₁₂-C₂₀ alkyl ether sulfosuccinates;
(C₆-C₂₄)acylisethionates and preferably (C₁₂-C_{IS})acylisethionates;
   in particular in the form of alkali metal or alkaline-earth metal, ammonium or amino alcohol salts.

The sulfate anionic surfactants that may be used include at least one sulfate function (—OSO₃H or —OSO₃⁻).

They may be chosen from the following compounds: alkyl sulfates, alkyl ether sulfates, alkylamido ether sulfates, alkylaryl polyether sulfates, monoglyceride sulfates; and the salts of these compounds;
the alkyl groups of these compounds including from 6 to 30 carbon atoms, particularly from 12 to 28, even better still from 14 to 24 or even from 16 to 22 carbon atoms; the aryl group preferably denoting a phenyl or benzyl group;
it being possible for these compounds to be polyoxyalkylenated, particularly polyoxyethylenated, and then preferably including from 1 to 50 ethylene oxide units and better still from 2 to 10 ethylene oxide units.

Preferentially, the sulfate anionic surfactants are chosen, alone or as a mixture, from:
in particular C₆-C₂₄ or even C₁₂-C₂₀ alkyl sulfates, and
in particular C₆-C₂₄ or even C₁₂-C₂₀ alkyl ether sulfates, preferably comprising from 1 to 20 ethylene oxide units;
   in particular in the form of alkali metal or alkaline-earth metal, ammonium or amino alcohol salts.

When the anionic surfactant is in salt form, said salt may be chosen from alkali metal salts, such as the sodium or potassium salt, ammonium salts, amine salts and in particular amino alcohol salts, and alkaline-earth metal salts, such as the magnesium salt. Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular sodium or magnesium salts are preferably used.

Preferentially, the anionic surfactants are chosen, alone or as a mixture, from:
C₆-C₂₄ and in particular C₁₂-C₂₀ alkyl sulfates;
C₆-C₂₄ and in particular C₁₂-C₂₀ alkyl ether sulfates; preferably comprising from 1 to 20 ethylene oxide units;
C₆-C₂₁ and in particular C₁₂-C₂₀ alkylsulfosuccinates;
C₆-C₂₄ and in particular C₁₂-C₂₀ olefin sulfonates;
C₆-C₂₄ and in particular C₁₂-C₂₀ alkyl ether sulfosuccinates;
(C₆-C₂₄)acylisethionates and preferably (C₁₂-C₁₈)acylisethionates;
C₆-C₂₄ and in particular C₁₂-C₂₀ acylsarcosinates;
(C₆-C₂₁)alkyl ether carboxylates, preferably (C₁₂-C₂₀)alkyl ether carboxylates; in particular those including from 2 to 50 ethylene oxide groups;
polyoxyalkylenated (C₆-C₂₄)alkylamido ether carboxylic acids and salts thereof, in particular those including from 2 to 50 alkylene oxide and in particular ethylene oxide groups;
C₆-C₂₄ and in particular C₁₂-C₂₀ acylglutamates;
C₆-C₂₄ and in particular C₁₂-C₂₀ acylglycinates;
   in particular in acid form or in the form of alkali metal or alkaline-earth metal, ammonium or amino alcohol salts.

Preferentially, the composition according to the invention comprises one or more anionic surfactants chosen from C₆-C₂₄ and in particular C₁₂-C₂₀ alkyl sulfates, C₆-C₂₄ and in particular C₁₂-C₂₀ alkyl ether sulfates preferably comprising from 1 to 20 ethylene oxide units; these surfactants more particularly being in the form of alkali metal or alkaline-earth metal, ammonium or amino alcohol salts; and mixtures thereof.

The composition according to the invention may also be free (0%) of sulfate surfactants (sulfate-free); in that case, it advantageously comprises one or more anionic surfactants chosen, alone or as a mixture, from
C₆-C₂₄ and in particular C₁₂-C₂₀ alkylsulfosuccinates;
C₆-C₂₄ and in particular C₁₂-C₂₀ olefinsulfonates,
C₆-C₂₄ and in particular C₁₂-C₂₀ alkyl ether sulfosuccinates;
(C₆-C₂₄)acylisethionates, preferably (C₁₂-C₁₈)acylisethionates,
C₆-C₂₄ and in particular C₁₂-C₂₀ acylsarcosinates;
(C₆-C₂₁)alkyl ether carboxylates, preferably (C₁₂-C₂₀)alkyl ether carboxylates; in particular those including from 2 to 50 ethylene oxide groups;
   in particular in acid form or in the form of alkali metal or alkaline-earth metal, ammonium or amino alcohol salts.

When they are present, the anionic surfactant(s) are preferably present in the composition according to the invention in a total amount ranging from 0.01% to 30% by weight, which may particularly range from 1% to 28% by weight, in particular from 5 to 25% by weight, preferably from 7% to 23% by weight, better still from 8% to 20% by weight, relative to the total weight of the composition.

### Amphoteric Surfactants

The composition according to the invention may further comprise at least one amphoteric surfactant.

Preferably, the composition according to the invention comprises one or more amphoteric surfactant(s).

The amphoteric surfactant(s) that can be used in the context of the invention are nonsilicone.

The term “amphoteric surfactant” means a surfactant including, as ionic or ionizable groups, one or more anionic groups and one or more cationic groups.

Mention may in particular be made, alone or as a mixture, of (C₈-C₂₀)alkylbetaines, (C₈-C₂₀)alkylsulfobetaines, (C₈-C₂₀)alkylamido(C₃-C₈)alkylbetaines and (C₈-C₂₀)alkylamido(C₆-C₈)alkylsulfobetaines.

They may also be chosen from optionally quaternized secondary or tertiary aliphatic amine derivatives, in which the aliphatic group is a linear or branched chain including from 8 to 22 carbon atoms, said amine derivatives containing at least one anionic group, for instance a carboxylate, sulfonate, sulfate, phosphate or phosphonate group.

Among the optionally quaternized secondary or tertiary aliphatic amine derivatives that may be used, as defined above, mention may also be made of the compounds having the respective structures (A), (B) and (C) below:

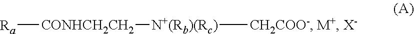

in which:
Rₐ represents a C₁₀ to C₃₀ alkyl or alkenyl group derived from an acid RₐCOOH preferably present in hydrolyzed coconut kernel oil, or a heptyl, nonyl or undecyl group;
R_{b} represents a β-hydroxyethyl group; and
R_{c} represents a carboxymethyl group;
M⁺ represents a cationic counterion derived from an alkali metal or alkaline-earth metal, such as sodium, an ammonium ion or an ion derived from an organic amine, and
X⁻ represents an organic or inorganic anionic counterion, such as that chosen from halides, acetates, phosphates, nitrates, (C₁-C₄)alkyl sulfates, (C₁-C₄)alkyl- or (C₁-C₄)alkylaryl-sulfonates, in particular methyl sulfate and ethyl sulfate; or alternatively M⁺ and X⁻ are absent;

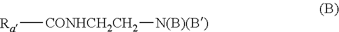

in which:
B represents the group —CH₂CH₂OX′;
B′ represents the group —(CH₂)_{z}Y′, with z=1 or 2;
X′ represents the group —CH₂COOH, —CH₂—COOZ′, —CH₂CH₂COOH or CH₂CH₂—COOZ′, or a hydrogen atom;
Y′ represents the group —COOH, —COOZ′ or —CH₂CH(OH)SO₃H or the group CH₂CH(OH)SO₃—Z′;
Z′ represents a cationic counterion derived from an alkali metal or alkaline-earth metal, such as sodium, an ammonium ion or an ion derived from an organic amine;
R_{a′} represents a C₁₀ to C₃₀ alkyl or alkenyl group of an acid R_{a′}—COOH which is preferably present in hydrolysed linseed oil or coconut kernel oil, an alkyl group, particularly a C₁₇ alkyl group, and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M Concentrate.

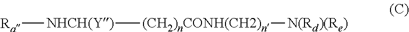

in which:
Y″ represents the group —COOH, —COOZ″ or —CH₂CH(OH)SO₃H or the group CH₂CH(OH)SO₃—Z″;
R_{d} and R_{e′} independently of each other, represent a C₁ to C₄ alkyl or hydroxyalkyl radical;
Z″ represents a cationic counterion derived from an alkali metal or alkaline-earth metal, such as sodium, an ammonium ion or an ion derived from an organic amine;
R_{a″} represents a C₁₀ to C₃₀ alkyl or alkenyl group of an acid R_{a″}COOH which is preferably present in hydrolysed linseed oil or coconut kernel oil;
n and n′ denote, independently of each other, an integer ranging from 1 to 3.

Among the amphoteric surfactants, use is preferably made of (C₈-C₂₀)alkylbetaines such as cocoylbetaine, (C₈-C₂₀)alkylamido(C₃-C₈)alkylbetaines such as cocamidopropylbetaine, and mixtures thereof, and the compounds of formula (C) such the salts, particularly the sodium salt, of diethylaminopropyl laurylaminosuccinamate (INCI name: sodium diethylaminopropyl cocoaspartamide).

Preferentially, the amphoteric surfactants are chosen from (C₈-C₂₀)alkylbetaines such as cocoylbetaine, (C₈-C₂₀)alkylamido(C₃-C₈)alkylbetaines such as cocamidopropylbetaine, and mixtures thereof.

When they are present, the amphoteric surfactant(s) are preferably present in the composition according to the invention in a total amount which may range from 0.01% to 15% by weight, better still from 0.1 to 12% by weight, particularly from 0.5% to 8% by weight, better still from 1% to 5% by weight, relative to the total weight of the composition.

### Cationic Surfactants

The composition according to the invention may further comprise at least one cationic surfactant.

Preferably, the composition according to the invention comprises one or more cationic surfactants.

Said cationic surfactants are non-silicone surfactants, that is to say that they do not contain any Si—O groups.

They are preferably chosen from quaternary ammonium salts, optionally polyoxyalkylenated primary, secondary or tertiary fatty amines, or salts thereof, and mixtures thereof.

The composition according to the invention may comprise one or more cationic surfactants chosen, alone or as a mixture, from the following compounds, which are quaternary ammonium salts:
the compounds corresponding to the general formula (II) below:

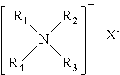

in which:
X⁻ is an anion, particularly chosen from the group of halides, phosphates, acetates, lactates, (C₁-C₄)alkyl sulfates, (C₁-C₄)alkylsulfonates or (C₁-C₄)alkylarylsulfonates; the groups R₁ to R₄, which may be identical or different, represent a linear or branched aliphatic group including from 1 to 30 carbon atoms, or an aromatic group such as aryl or alkylaryl, at least one of the groups R₁ to R₄ denoting a linear or branched aliphatic group including from 8 to 30 carbon atoms, preferably from 12 to 24 carbon atoms.

The aliphatic groups may comprise heteroatoms, particularly such as oxygen, nitrogen, sulfur and halogens. The aliphatic groups are chosen, for example, from C₁-C₃₀ alkyl, C₁-C₃₀ alkoxy, (C₂—C) polyoxyalkylene, C₁-C₃₀ alkylamide, (C₁₂-C₂₂)alkylamido(C₂-C₆)alkyl, (C₁₂-C₂₂)alkyl acetate, and C₁-C₃₀ hydroxyalkyl groups.

Among the quaternary ammonium salts of formula (II), preference is given to tetraalkylammonium salts, for instance dialkyldimethylammonium or alkyltrimethylammonium salts, in which the alkyl group includes from approximately 12 to 22 carbon atoms, in particular behenyltrimethylammonium, stearyltrimethylammonium, distearyldimethylammonium, cetyltrimethylammonium, dicetyldimethylammonium, or benzyldimethylstearylammonium salts, and also palmitylamidopropyltrimethylammonium salts, stearamidopropyltrimethylammonium salts, stearamidopropyldimethylcetearylammonium salts, or stearamidopropyldimethyl(myristyl acetate)ammonium salts such as those sold under the name Ceraphyl® 70 by the company Van Dyk.

It is preferred in particular to use the chloride, bromide or methyl sulfate salts of these compounds.
the quaternary ammonium salts of imidazoline, such as those of formula (III):

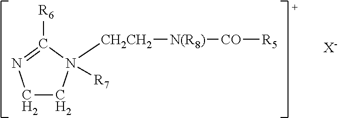

in which
R₆ represents an alkenyl or alkyl group including from 8 to 30 carbon atoms, for example derived from tallow fatty acids,
R₆ represents a hydrogen atom, a C₁-C₄ alkyl group or an alkenyl or alkyl group including from 8 to 30 carbon atoms,
R₇ represents a C₁-C₄ alkyl group,
R₈ represents a hydrogen atom or a C₁-C₄ alkyl group,
X⁻ is an anion chosen from the group of halides, phosphates, acetates, lactates, alkyl sulfates, alkyl- or alkylaryl-sulfonates in which the alkyl and aryl groups preferably comprise, respectively, from 1 to 20 carbon atoms and from 6 to 30 carbon atoms.

Preferably, R₅ and R₆ denote a mixture of alkenyl or alkyl groups including from 12 to 21 carbon atoms, for example derived from tallow fatty acids, R₇ denotes a methyl group and R₈ denotes a hydrogen atom.

Such a product is sold, for example, under the name Rewoquat® W 75 by the company Rewo.
quaternary diammonium or triammonium salts, in particular of formula (IV):

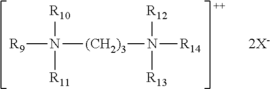

in which
R₉ denotes an alkyl radical including from approximately 16 to 30 carbon atoms which is optionally hydroxylated and/or optionally interrupted with one or more oxygen atoms,
R₁₀ is chosen from hydrogen or an alkyl radical including from 1 to 4 carbon atoms or a group (R₉ₐ)(R₁₀ₐ)(R₁₁ₐ)N—(CH₂)₃, with R₉ₐ, R₁₀ₐ, R₁₁ₐ, R₁₁, R₁₂, R₁₃ and R₁₄, which are identical or different, being chosen from hydrogen or an alkyl radical including from 1 to 4 carbon atoms, and
X⁻ is an anion chosen from the group of halides, acetates, phosphates, nitrates, (C₁-C₄)alkyl sulfates, (C₁-C₄)alkylsulfonates and (C₁-C₄)alkylarylsulfonates, in particular methyl sulfate and ethyl sulfate.

Such compounds are, for example, Finquat CT-P, sold by the company Finetex (Quaternium 89), and Finquat CT, sold by the company Finetex (Quaternium 75).
quaternary ammonium salts containing at least one ester function, such as those of formula (V) below:

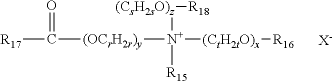

in which:
R₁₅ is chosen from C₁-C₆ alkyl groups and C₁-C₆ hydroxyalkyl or dihydroxyalkyl groups;
R₁₆ is chosen from the group R₁₉—C(O)—; groups R₂₀ which are linear or branched, saturated or unsaturated C₁-C₂₂ hydrocarbon-based groups; a hydrogen atom;
R₁₈ is chosen from the group R21-C(O)—; groups R₂₂ which are linear or branched, saturated or unsaturated C₁-C₆ hydrocarbon-based groups; a hydrogen atom;
R₁₇, R₁₉, and R₂₁, which are identical or different, are chosen from linear or branched, saturated or unsaturated C₇-C₂₁ hydrocarbon-based groups;
r, s and t, which are identical or different, are integers ranging from 2 to 6;
y is an integer ranging from 1 to 10;
x and z, which are identical or different, are integers ranging from 0 to 10;
X⁻ is a simple or complex, organic or inorganic anion;
with the proviso that the sum x+y+z is from 1 to 15, that when x is 0 then R₁₆ denotes R₂₀, and that when z is 0 then R₁₈ denotes R₂₂.

The alkyl groups R₁₅ may be linear or branched, and more particularly linear. Preferably, R₁₅ denotes a methyl, ethyl, hydroxyethyl or dihydroxypropyl group, and more particularly a methyl or ethyl group.

Advantageously, the sum x+y+z is from 1 to 10.

When R₁₆ is a hydrocarbon-based group R₂₀, it may be long and contain from 12 to 22 carbon atoms, or may be short and contain from 1 to 3 carbon atoms.

When R₁₈ is a hydrocarbon-based group R₂₂, it preferably contains 1 to 3 carbon atoms.

Advantageously, R₁₇, R₁₉ and R₂₁, which are identical or different, are chosen from linear or branched, saturated or unsaturated C₁₁-C₂₁ hydrocarbon-based groups, and more particularly from linear or branched, saturated or unsaturated C₁₁-C₂₁ alkyl and alkenyl groups.

Preferably, x and z, which are identical or different, are equal to 0 or 1.

Advantageously, y is equal to 1.

Preferably, r, s and t, which are identical or different, are equal to 2 or 3, and even more particularly are equal to 2.

The anion X is preferably a halide (chloride, bromide or iodide) or an alkyl sulfate, more particularly methyl sulfate. However, use may be made of methanesulfonate, phosphate, nitrate, tosylate, an anion derived from an organic acid, such as acetate or lactate, or any other anion that is compatible with ammonium bearing an ester function.

The anion X⁻ is even more particularly chloride or methyl sulfate.

Use may be made more particularly, in the composition according to the invention, of the ammonium salts of formula (V) in which
R₁₅ denotes a methyl or ethyl group,
x and y are equal to 1; z is equal to 0 or 1;
r, s and t are equal to 2;
R₁₆ is chosen from the group R₁₉—C(O)—; methyl, ethyl or C₁₁-C₂₂ hydrocarbon-based groups, and a hydrogen atom;
R₁₈ is chosen from the group R₂₁—C(O)— and a hydrogen atom,
R₁₇, R₁₉ and R₂₁, which are identical or different, are chosen from linear or branched, saturated or unsaturated C₁₃-C₁₇ hydrocarbon-based groups, and preferably from linear or branched, saturated or unsaturated C₁₃-C₁₇ alkyl and alkenyl groups.

Advantageously, the hydrocarbon-based groups are linear.

Mention may be made, for example, of the compounds of formula (V) such as the diacyloxyethyldimethylammonium, diacyloxyethylhydroxyethylmethylammonium, monoacyloxyethyldihydroxyethylmethylammonium, triacyloxyethylmethylammonium and monoacyloxyethylhydroxyethyldimethylammonium salts (particularly chloride or methyl sulfate), and mixtures thereof. The acyl groups preferably have 14 to 18 carbon atoms and originate more particularly from a plant oil such as palm oil or sunflower oil. When the compound contains several acyl groups, these groups may be identical or different. These products are obtained, for example, by direct esterification of triethanolamine, triisopropanolamine, alkyldiethanolamine or alkyldiisopropanolamine, which are optionally oxyalkylenated, with C₁₀-C₃₀ fatty acids or with mixtures of C₁₀-C₃₀ fatty acids of plant or animal origin, or by transesterification of the methyl esters thereof. This esterification is followed by a quaternization using an alkylating agent such as an alkyl halide (preferably a methyl or ethyl halide), a dialkyl sulfate (preferably a methyl or ethyl sulfate), methyl methanesulfonate, methyl para-toluenesulfonate, glycol chlorohydrin or glycerol chlorohydrin.

Such compounds are sold, for example, under the names Dehyquart® by the company Henkel, Stepanquat® by the company Stepan, Noxamium® by the company CECA or Rewoquat® WE 18 by the company Rewo-Witco.

The composition according to the invention may contain, for example, a mixture of quaternary ammonium monoester, diester and triester salts with a weight majority of diester salts.

Use may also be made of the ammonium salts containing at least one ester function that are described in patents U.S. Pat. Nos. 4,874,554 and 4,137,180.

Use may be made of behenoylhydroxypropyltrimethylammonium chloride sold by Kao under the name Quatarmin BTC 131.

Preferably, the ammonium salts containing at least one ester function contain two ester functions.

Among the quaternary ammonium salts containing at least one ester function that may be used, it is preferred to use dipalmitoylethylhydroxyethylmethylammonium salts.

The term “fatty amine” means a compound comprising at least one optionally (poly)oxyalkylenated primary, secondary or tertiary amine function, or salts thereof and comprising at least one C₈-C₃₀ and preferably C₈-C₀ hydrocarbon-based chain.

Preferably, the fatty amines which are useful according to the invention are not (poly)oxyalkylenated.

Fatty amines that may be mentioned include amidoamines. The amidoamines according to the invention may be chosen from fatty amidoamines, it being possible for the fatty chain to be borne by the amine group or by the amido group.

The term “amidoamine” means a compound comprising at least one amide function and at least one primary, secondary or tertiary amine function.

The term “fatty amidoamine” means an amidoamine comprising, in general, at least one C₆-C₃₀ hydrocarbon-based chain. Preferably, the fatty amidoamines which are useful according to the invention are not quaternized.

Preferably, the fatty amidoamines which are useful according to the invention are not (poly)oxyalkylenated.

Among the fatty amidoamines which are useful according to the invention, mention may be made of the amidoamines of formula (VI) below:

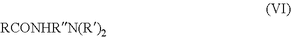

in which:
R represents a substituted or unsubstituted, linear or branched, saturated or unsaturated monovalent hydrocarbon-based radical containing from 5 to 29 carbon atoms, preferably from 7 to 23 carbon atoms, and in particular a linear or branched C₅-C₂₉ and preferably C₇-C₂₃ alkyl radical, or a linear or branched C₅-C₉ and preferably C₇-C₂₃ alkenyl radical;
R″ represents a divalent hydrocarbon-based radical containing less than 6 carbon atoms, preferably from 2 to 4 carbon atoms and better still 3 carbon atoms; and
R′, which are identical or different, represent a substituted or unsubstituted, saturated or unsaturated, linear or branched, monovalent hydrocarbon-based radical containing less than 6 carbon atoms, preferably from 1 to 4 carbon atoms, preferably a methyl radical.

The fatty amidoamines of formula (VI) are chosen, for example, from oleamidopropyldimethylamine, stearamidopropyldimethylamine, particularly the product sold by the company Inolex Chemical Company under the name Lexamine S13, isostearamidopropyldimethylamine, stearamidoethyldimethylamine, lauramidopropyldimethylamine, myristamidopropyldimethylamine, behenamidopropyldimethylamine, dilinoleamidopropyldimethylamine, palmitamidopropyldimethylamine, ricinoleamindopropyldimethylamine, soyamidopropyldimethylamine, avocadoamidopropyldimethylamine, cocamidopropyldimethylamine, minkamidopropyldimethylamine, oatamidopropyldimethylamine, sesamidopropyldimethylamine, tallamidopropyldimethylamine, olivamidopropyldimethylamine, palmitamidopropyldimethylamine, stearamidoethyldiethylamine, brassicamidopropyldimethylamine and mixtures thereof.

Preferably, the fatty amidoamines are chosen from oleamidopropyldimethylamine, stearamidopropyldimethylamine, brassicamidopropyldimethylamine and mixtures thereof.

The cationic surfactant(s) are preferably chosen from those of formula (II) above, those of formula (V) above, those of formula (VI) above, and mixtures thereof; better still from those of formula (II) above, those of formula (VI) above, and mixtures thereof; even better still from those of formula (II) above.

Preferentially, the cationic surfactant(s) may be chosen from salts such as chlorides, bromides or methosulfates, of tetraalkylammonium, for instance dialkyldimethylammonium or alkyltrimethylammonium salts in which the alkyl group includes from approximately 12 to 22 carbon atoms, in particular behenyltrimethylammonium, stearyltrimethylammonium, distearyldimethylammonium, cetyltrimethylammonium, dicetyldimethylammonium or benzyldimethylstearylammonium salts; dipalmitoylethylhydroxyethylmethylammonium salts such as dipalmitoylethylhydroxyethylmethylammonium methosulfate; and mixtures thereof.

Even more preferentially, they are chosen from cetyltrimethylammonium chloride, behenyltrimethylammonium chloride, dipalmitoylethylhydroxyethylmethylammonium methosulfate, and mixtures thereof.

When they are present, the total content of cationic surfactant(s) in the composition according to the invention preferably ranges from 0.1% to 10% by weight, particularly from 0.2% to 8% by weight, better still from 0.3% to 7% by weight, even better still from 0.5% to 5% by weight, relative to the total weight of the composition.

### Nonionic Surfactants

The composition according to the invention may further comprise at least one nonionic surfactant.

Preferably, the composition according to the invention comprises one or more nonionic surfactants.

Said nonionic surfactants may be chosen from:
alcohols, α-diols and (C₁-C₂₀)alkylphenols, these compounds being polyethoxylated and/or polypropoxylated and/or polyglycerolated, it being possible for the number of ethylene oxide and/or propylene oxide groups to range from 1 to 100, and the number of glycerol groups to range from 2 to 30; or else these compounds comprising at least one fatty chain including from 8 to 40 carbon atoms and particularly from 16 to 30 carbon atoms; in particular, oxyethylenated alcohols comprising at least one saturated or unsaturated, linear or branched C₈ to C₄₀ alkyl chain, comprising from 1 to 100 mol of ethylene oxide, preferably from 2 to 50 and more particularly from 2 to 40 mol of ethylene oxide and including one or two fatty chains;
condensates of ethylene oxide and propylene oxide with fatty alcohols;
polyethoxylated fatty amides preferably containing from 2 to 30 ethylene oxide units, polyglycerolated fatty amides including on average from 1 to 5 and in particular from 1.5 to 4 glycerol groups;
ethoxylated fatty acid esters of sorbitan (or oxyethylenated sorbitan ester), preferably having from 2 to 40 ethylene oxide units;
fatty acid esters of sucrose;
polyoxyalkylenated, preferably polyoxyethylenated, fatty acid esters having from 2 to 150 mol of ethylene oxide, including oxyethylenated plant oils;
N—(C₆-C₂₄ alkyl)glucamine derivatives;
amine oxides such as (C10-C14 alkyl)amine oxides or N—(C₁₀-C₁₄ acyl)aminopropylmorpholine oxides;
nonionic surfactants of alkyl(poly)glycoside type.

Nonionic surfactants of alkyl(poly)glycoside type may be represented by the following general formula:

R₁O—(R₂O)ₜ-(G)ᵥ

in which:
R1 represents a linear or branched alkyl or alkenyl radical including 6 to 24 carbon atoms and particularly 8 to 18 carbon atoms, or an alkylphenyl radical of which the linear or branched alkyl radical includes 6 to 24 carbon atoms and particularly 8 to 18 carbon atoms,
R2 represents an alkylene radical including 2 to 4 carbon atoms;
G represents a sugar unit including 5 to 6 carbon atoms;
t denotes a value ranging from 0 to 10 and preferably from 0 to 4;
v denotes a value ranging from 1 to 15 and preferably from 1 to 4.

Preferably, the alkyl(poly)glycoside surfactants are of the formula described above in which:
R1 denotes a linear or branched, saturated or unsaturated alkyl radical including from 8 to 18 carbon atoms,
R2 represents an alkylene radical including 2 to 4 carbon atoms;
t denotes a value ranging from 0 to 3 and preferably equal to 0,
G denotes glucose, fructose or galactose, preferably glucose,
it being possible for the degree of polymerization, i.e. the value of v, to range from 1 to 15 and preferably from 1 to 4; the mean degree of polymerization more particularly being between 1 and 2.

The glucoside bonds between the sugar units are generally of 1-6 or 1-4 type and preferably of 1-4 type. Preferably, the alkyl (poly)glycoside surfactant is an alkyl (poly)glucoside surfactant. 1,4 C₈/C₁₆ alkyl(poly)glucosides, and particularly decyl glucosides and caprylyl/capryl glucosides, are most particularly preferred.

The oxyethylenated sorbitan esters that can be used in the context of the invention particularly comprise the oxyethylenated derivatives of C₈-C₃₀ fatty acid monoesters and polyesters of sorbitan, having from 2 to 40 ethylene oxide units. Use is preferably made of the oxyethylenated derivatives of C₁₂-C₂₄ fatty acid monoesters and polyesters of sorbitan, having from 4 to 20 ethylene oxide units.

Such compounds also known under the name of polysorbates. They are, inter alia, sold under the name Tween by the company Uniqema.

Mention may particularly be made of oxyethylene sorbitan monolaurate with 4 EO, for example sold under the name TWEEN 21, oxyethylene sorbitan monolaurate with 20 EO, for example sold under the name TWEEN 20, oxyethylene sorbitan monopalmitate with 20 EO, for example sold under the name TWEEN 40, oxyethylene sorbitan monostearate with 20 EO, for example sold under the name TWEEN 60, oxyethylene sorbitan monostearate with 4 EO, for example sold under the name TWEEN 61, oxyethylene sorbitan tristearate with 20 EO, for example sold under the name TWEEN 65, oxyethylene sorbitan monooleate with 20 EO, for example sold under the name TWEEN 80, oxyethylene sorbitan monooleate with 5 EO, for example sold under the name TWEEN 81, oxyethylene sorbitan trioleate with 20 EO, for example sold under the name TWEEN 85.

In the present document, and in a manner well known per se, a “compound with X EO” denotes an oxyethylenated compound comprising X oxyethylene units per molecule.

Preferably, the fatty acid of the oxyethylenated sorbitan ester is a saturated fatty acid, in particular a C₁₂-C₂₄ saturated fatty acid.

The preferred sorbitan esters are the oxyethylenated derivatives of C₁₂-C₂₄ fatty acid monoesters of sorbitan, having from 4 to 20 ethylene oxide units, more preferentially oxyethylene sorbitan monolaurate with 4 EO, oxyethylene sorbitan monolaurate with 20 EO, oxyethylene sorbitan monostearate with 20 EO, and mixtures thereof.

Preferentially, the nonionic surfactants are chosen from, alone or as a mixture, (C₆-C₂₄ alkyl)(poly)glycosides, and more particularly (C₈-C₁₈ alkyl)(poly)glycosides; and oxyethylenated sorbitan esters, in particular those derived from C₁₂-C₂₄ saturated fatty acids and comprising 4 to 20 ethylene oxide units.

Preferably, the nonionic surfactant(s) may be present in the composition according to the invention in a total content ranging from 0.01% to 10% by weight, preferentially ranging from 0.05% to 8% by weight, in particular ranging from 0.1% to 5% by weight, and even better still from 0.2% to 2% by weight, relative to the total weight of the composition.

### Cationic Polymers

The composition according to the invention may optionally comprise one or more nonsilicone polymers which may be chosen from anionic polymers, nonionic polymers, amphoteric polymers and cationic polymers.

Preferably, the composition according to the invention comprises one or more cationic polymers.

Advantageously, the polymer(s), particularly cationic polymer(s), are other than associative polymers (they are therefore non-associative).

The term “cationic polymer” denotes any non-silicone (not comprising any silicon atoms) polymer containing cationic groups and/or groups that can be ionized into cationic groups and not containing any anionic groups and/or groups that can be ionized into anionic groups.

The cationic polymers that may be employed preferably have a cationic charge density of less than or equal to 5 milliequivalents/gram (meq/g), better still of less than or equal to 4 meq/g.

The cationic charge density of a polymer corresponds to the number of moles of cationic charges per unit mass of polymer under the conditions in which the latter is completely ionized. It may be determined by calculation if the structure of the polymer is known, i.e. the structure of the monomers constituting the polymer and their molar proportion or weight proportion. It may also be determined experimentally by the Kjeldahl method.

The cationic polymers that may be used preferably have a weight-average molar mass (Mw) of between 500 and 5×10⁶ approximately and preferably between 10³ and 3×10⁶ approximately.

Among the cationic polymers that may be used, mention may be made of:

(1) homopolymers or copolymers derived from acrylic or methacrylic esters or amides and including at least one of the units having the following formulae:

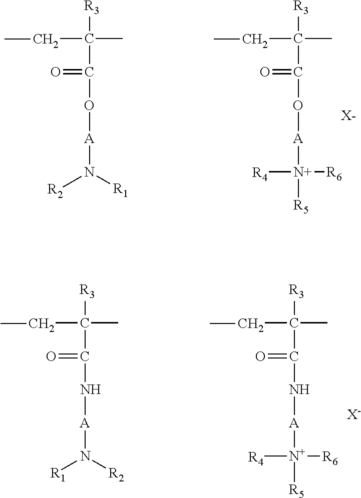

in which:
R3, which are identical or different, denote a hydrogen atom or a CH₃ radical;
A, which are identical or different, represent a linear or branched divalent alkyl group of 1 to 6 carbon atoms, preferably 2 or 3 carbon atoms, or a hydroxyalkyl group of 1 to 4 carbon atoms;
R4, R5 and R6, which are identical or different, represent an alkyl group having from 1 to 18 carbon atoms or a benzyl radical, preferably an alkyl group having from 1 to 6 carbon atoms;
R1 and R2, which are identical or different, represent a hydrogen atom or an alkyl group having from 1 to 6 carbon atoms, preferably methyl or ethyl;
X denotes an anion derived from a mineral or organic acid, such as a methosulfate anion or a halide such as chloride or bromide.

The copolymers of family (1) may also contain one or more units deriving from comonomers that may be chosen from the family of the acrylamides, methacrylamides, diacetone acrylamides, acrylamides and methacrylamides substituted on the nitrogen with lower (C₁-C₄) alkyls, acrylic or methacrylic acid esters, vinyllactams such as vinylpyrrolidone or vinylcaprolactam, and vinyl esters.

Among these copolymers of family (1), mention may be made of:
copolymers of acrylamide and of dimethylaminoethyl methacrylate quaternized with dimethyl sulfate or with a dimethyl halide, such as the product sold under the name Hercofloc by the company Hercules,
copolymers of acrylamide and of methacryloyloxyethyltrimethylammonium chloride, such as the products sold under the name Bina Quat P 100 by the company Ciba Geigy,
the copolymer of acrylamide and of methacryloyloxyethyltrimethylammonium methosulfate, such as the product sold under the name Reten by the company Hercules,
quaternized or non-quaternized vinylpyrrolidone/dialkylaminoalkyl acrylate or methacrylate copolymers, such as the products sold under the name Gafquat by the company ISP, for instance Gafquat 734 or Gafquat 755, or alternatively the products known as Copolymer 845, 958 and 937,
dimethylaminoethyl methacrylate/vinylcaprolactam/vinylpyrrolidone terpolymers, such as the product sold under the name Gaffix VC 713 by the company ISP,
vinylpyrrolidone/methacrylamidopropyldimethylamine copolymers, such as the products sold under the name Styleze CC 10 by ISP;
quaternized vinylpyrrolidone/dimethylaminopropylmethacrylamide copolymers such as the product sold under the name Gafquat HS 100 by the company ISP;
polymers, preferably crosslinked polymers, of methacryloyloxy(C₁-C₄)alkyl tri(C₁-C₄)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with an olefinically unsaturated compound, in particular methylenebisacrylamide. Use may be made more particularly of a crosslinked acrylamide/methacryloyloxyethyltrimethylammonium chloride copolymer (20/80 by weight) in the form of a dispersion comprising 50% by weight of said copolymer in mineral oil. This dispersion is sold under the name Salcare® SC 92 by the company Ciba.

Use may also be made of a crosslinked methacryloyloxyethyltrimethylammonium chloride homopolymer comprising approximately 50% by weight of the homopolymer in mineral oil or in a liquid ester. These dispersions are sold under the names Salcare® SC 95 and Salcare® SC 96 by the company Ciba.

(2) cationic polysaccharides, particularly cationic celluloses and galactomannan gums. Among the cationic polysaccharides, mention may be made more particularly of cellulose ether derivatives including quaternary ammonium groups, cationic cellulose copolymers or cellulose derivatives grafted with a water-soluble quaternary ammonium monomer and cationic galactomannan gums.

The cellulose ether derivatives including quaternary ammonium groups are particularly described in FR1492597; they are also defined in the CTFA dictionary as quaternary ammoniums of hydroxyethylcellulose that has reacted with an epoxide substituted with a trimethylammonium group.

Mention may particularly be made of the polymers sold under the name Ucare Polymer JR (JR 400 LT, JR 125 and JR 30M) or LR (LR 400 and LR 30M) by the company Amerchol.

Cationic cellulose copolymers and cellulose derivatives grafted with a water-soluble quaternary ammonium monomer are described particularly in patent U.S. Pat. No. 4,131,576; mention may be made of hydroxyalkyl celluloses, for instance hydroxymethyl, hydroxyethyl or hydroxypropyl celluloses particularly grafted with a methacryloylethyltrimethylammonium, methacrylamidopropyltrimethylammonium or dimethyldiallylammonium salt. Mention may be made most particularly of crosslinked or non-crosslinked quaternized hydroxyethylcelluloses, it being possible for the the quaternizing agent to particularly be diallyldimethylammonium chloride; and most particularly hydroxypropyltrimethylammonium hydroxyethylcellulose.

Among the commercial products corresponding to this definition, mention may be made of the products sold under the names Celquat L 200 and Celquat H 100 by the company National Starch.

A particularly preferred cationic cellulose that may particularly be mentioned is the polymer having the INCI name Polyquaternium-10.

The cationic galactomannan gums are particularly described in patents U.S. Pat. Nos. 3,589,578 and 4,031,307; mention may be made of cationic guar gums, particularly those comprising cationic trialkylammonium groups, particularly trimethylammonium. Mention may thus be made of guar gums modified with a 2,3-epoxypropyltrimethylammonium salt (for example a chloride).

Preferably, 2% to 30% by number of the hydroxyl functions of the guar gums bear cationic trialkylammonium groups. Even more preferentially, 5% to 20% by number of the hydroxyl functions of these guar gums are branched with cationic trialkylammonium groups. Among these trialkylammonium groups, mention may most particularly be made of the trimethylammonium and triethylammonium groups. Even more preferentially, these groups represent from 5% to 20% by weight relative to the total weight of the modified guar gum. According to the invention, use may be made of guar gums modified with 2,3-epoxypropyltrimethylammonium chloride.

Mention may be made in particular of the products having the INCI names Hydroxypropyl guar hydroxypropyltrimonium chloride and Guar hydroxypropyltrimonium chloride. Such products are particularly sold under the names Jaguar C13S, Jaguar C15, Jaguar C17 and Jaguar C162 by the company Solvay.

Among the cationic polysaccharides that may be used, mention may also be made of cationic derivatives of cassia gum, particularly those including quaternary ammonium groups; in particular, mention may be made of the product having the INCI name Cassia hydroxypropyltrimonium chloride.

(3) polymers formed from piperazinyl units and divalent alkylene or hydroxyalkylene radicals containing linear or branched chains, optionally interrupted with oxygen, sulfur or nitrogen atoms or with aromatic or heterocyclic rings, and also the oxidation and/or quaternization products of these polymers.

(4) water-soluble polyaminoamides prepared in particular by polycondensation of an acidic compound with a polyamine; these polyaminoamides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyaminoamide; these polyaminoamides can be alkylated or, if they include one or more tertiary amine functions, they can be quaternized.

(5) polyaminoamide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl radical includes from 1 to 4 carbon atoms and preferably denotes methyl, ethyl or propyl. Among these derivatives, mention may be made more particularly of the adipic acid/dimethylaminohydroxypropyl/diethylenetriamine polymers sold under the name Cartaretine F, F4 or F8 by the company Sandoz.

(6) polymers obtained by reacting a polyalkylene polyamine including two primary amine groups and at least one secondary amine group with a dicarboxylic acid chosen from diglycolic acid and saturated aliphatic dicarboxylic acids having from 3 to 8 carbon atoms; the mole ratio between the polyalkylene polyamine and the dicarboxylic acid preferably being between 0.8:1 and 1.4:1; the resulting polyaminoamide being reacted with epichlorohydrin in a mole ratio of epichlorohydrin relative to the secondary amine group of the polyaminoamide preferably of between 0.5:1 and 1.8:1. Polymers of this type are sold in particular under the name Hercosett 57 by the company Hercules Inc. or under the name PD 170 or Delsette 101 by the company Hercules in the case of the adipic acid/epoxy-propyl/diethylenetriamine copolymer.

(7) cyclopolymers of alkyldiallylamine or of dialkyldiallylammonium, such as the homopolymers or copolymers including, as main constituent of the chain, units corresponding to formula (I) or (II):

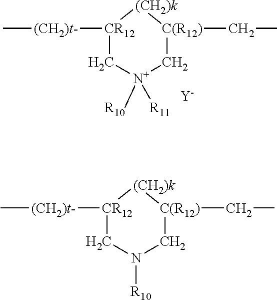

in which
k and t are equal to 0 or 1, the sum k+t being equal to 1;
R₁₂ denotes a hydrogen atom or a methyl radical;
R₁₀ and R₁₁, independently of one another, denote a C₁-C₆ alkyl group, a C₁-C₅ hydroxyalkyl group, a C₁-C₄ amidoalkyl group; or alternatively R₁₀ and R₁₁ may denote, together with the nitrogen atom to which they are attached, a heterocyclic group such as piperidinyl or morpholinyl; R₁₀ and R₁₁, independently of one another, preferably denote a C₁-C₄ alkyl group;
Y⁻ is an anion such as bromide, chloride, acetate, borate, citrate, tartrate, bisulfate, bisulfite, sulfate or phosphate.

Mention may be made more particularly of the homopolymer of dimethyldiallylammonium salts (for example chloride) (INCI name polyquaternium-6) for example sold under the name Merquat 100 by the company Nalco and the copolymers of diallyldimethylammonium salts (for example chloride) and of acrylamide (INCI name polyquaternium-7), particularly sold under the name Merquat 550 or Merquat 7SPR;

(8) quaternary diammonium polymers comprising repeating units of formula:

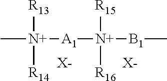

in which:
R₁₃, R₁₄, R₁₅ and R₁₆, which are identical or different, represent aliphatic, alicyclic or arylaliphatic radicals comprising from 1 to 20 carbon atoms or C₁-C₁₂ hydroxyalkyl aliphatic radicals;
   or else R₁₃, R₁₄, R₁₅ and R₁₆, together or separately, form, with the nitrogen atoms to which they are attached, heterocycles optionally comprising a second heteroatom other than nitrogen;
   or else R₁₃, R₁₄, R₁₅ and R₁₆ represent a linear or branched C₁-C₆ alkyl radical substituted with a nitrile, ester, acyl, amide or —CO—O—R₁₇-D or —CO—NH—R₁₇-D group, where R₁₇ is an alkylene and D is a quaternary ammonium group;
A1 and B1 represent linear or branched, saturated or unsaturated, divalent polymethylene groups comprising from 2 to 20 carbon atoms, which may contain, linked to or intercalated in the main chain, one or more aromatic rings or one or more oxygen or sulfur atoms or sulfoxide, sulfone, disulfide, amino, alkylamino, hydroxyl, quaternary ammonium, ureido, amide or ester groups, and
X⁻ denotes an anion derived from a mineral or organic acid;
   it being understood that A₁, R₁₃ and R₁₅ can form, with the two nitrogen atoms to which they are attached, a piperazine ring;
   in addition, if A1 denotes a linear or branched, saturated or unsaturated alkylene or hydroxyalkylene radical, B1 may also denote a group (CH₂)n-CO-D-OC—(CH₂)p- with n and p, which are identical or different, being integers ranging from 2 to 20, and D denoting:
   a) a glycol residue of formula —O—Z—O—, in which Z denotes a linear or branched hydrocarbon-based radical or a group corresponding to one of the following formulae: —(CH₂CH₂O)ₓ—CH₂CH₂— and —[CH₂CH(CH₃)O]_{y}—CH₂CH(CH₃)—, where x and y denote an integer from 1 to 4, representing a defined and unique degree of polymerization or any number from 1 to 4 representing an average degree of polymerization;
   b) a bis-secondary diamine residue, such as a piperazine derivative;
   c) a bis-primary diamine residue of formula —NH—Y—NH—, in which Y denotes a linear or branched hydrocarbon-based radical, or else the divalent radical —CH₂—CH₂—S—S—CH₂—CH₂—;
   d) a ureylene group of formula —NH—CO—NH—.

Preferably, X is an anion such as chloride or bromide.

These polymers have a number-average molar mass (Mn) generally of between 1000 and 100 000.

Mention may be made more particularly of polymers which are constituted of repeating units corresponding to the formula:

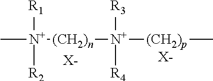

in which R₁, R₂, R₃ and R₄, which are identical or different, denote an alkyl or hydroxyalkyl radical having from 1 to 4 carbon atoms, n and p are integers ranging from 2 to 20, and
X⁻ is an anion derived from a mineral or organic acid.

A particularly preferred compound is that for which R₁, R₂, R₃ and R₄ represent a methyl radical and n=3, p=6 and X=Cl, which is known as Hexadimethrine chloride according to the INCI (CTFA) nomenclature.

(9) polyquaternary ammonium polymers comprising units of formula:

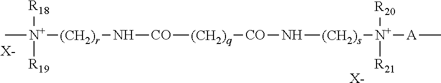

in which:
R₁₈, R₁₉, R₂₀ and R₂₁, which are identical or different, represent a hydrogen atom or a methyl, ethyl, propyl, β-hydroxyethyl, β-hydroxypropyl or —CH₂CH₂(OCH₂CH₂)pOH radical, where p is equal to 0 or to an integer of between 1 and 6, with the proviso that R₁₈, R₁₉, R₂₀ and R₂₁ do not simultaneously represent a hydrogen atom,
r and s, which are identical or different, are integers between 1 and 6,
q is equal to 0 or to an integer between 1 and 34,
X⁻ denotes an anion, such as a halide,
A denotes a divalent dihalide radical or preferably represents —CH₂—CH₂—O—CH₂—CH₂—.

Examples that may be mentioned include the products Mirapol® A 15, Mirapol® AD1, Mirapol® AZ1 and Mirapol® 175 sold by the company Miranol.

(10) quaternary polymers of vinylpyrrolidone and of vinylimidazole, for instance the products sold under the names Luviquat® FC 905, FC 550 and FC 370 by the company BASF.

(11) polyamines such as Polyquart® H sold by Cognis, which is referenced under the name “Polyethylene Glycol (15) Tallow Polyamine” in the CTFA dictionary.

(12) polymers including in their structure:
(a) one or more units corresponding to formula (A) below:

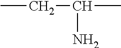

(b) optionally one or more units corresponding to formula (B) below:

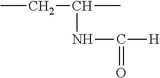

In other words, these polymers may be particularly chosen from homopolymers or copolymers including one or more units derived from vinylamine and optionally one or more units derived from vinylformamide.

Preferably, these cationic polymers are chosen from polymers including, in their structure, from 5 mol % to 100 mol % of units corresponding to formula (A) and from 0 to 95 mol % of units corresponding to formula (B), preferentially from 10 mol % to 100 mol % of units corresponding to formula (A) and from 0 to 90 mol % of units corresponding to formula (B).

These polymers may be obtained, for example, by partial hydrolysis of polyvinylformamide. This hydrolysis may take place in acidic or basic medium.

The weight-average molecular mass of said polymer, measured by light scattering, may range from 1000 to 3 000 000 g/mol, preferably from 10 000 to 1 000 000 and more particularly from 100 000 to 500 000 g/mol.

The polymers including units of formula (A) and optionally units of formula (B) are particularly sold under the name Lupamin by the company BASF; for instance, in a nonlimiting manner, the products sold under the names Lupamin 9095, Lupamin 5095, Lupamin 1095, Lupamin 9030 (or Luviquat 9030) and Lupamin 9010.

Preferably, the cationic polymers that may be employed in the context of the invention are chosen, alone or as a mixture, from the polymers of family (1) and in particular the polymers, preferably crosslinked, of methacryloyloxyalkyl (C₁-C₄)alkyltri(C₁-C₄)alkylammonium salts, such as Polyquaternium-7, and cationic polysaccharides, particularly cationic celluloses, such as Polyquaternium-10; and cationic galactomannan gums, particularly cationic guar gums; and also mixtures thereof.

When they are present, the composition according to the invention may comprise the polymer(s) in a total amount ranging from 0.01% to 10% by weight, better still from 0.05% to 5% by weight, even better still from 0.1% to 2% by weight, relative to the total weight of the composition.

When they are present, the composition according to the invention may comprise the cationic polymer(s) in a total amount ranging from 0.01% to 10% by weight, better still from 0.05% to 5% by weight, even better still from 0.1% to 2% by weight, relative to the total weight of the composition.

### Additional Silicones

The composition according to the invention may also comprise at least one additional silicone.

Preferably, the composition according to the invention comprises one or more additional silicones.

The additional silicone(s) may in particular be chosen from amino silicones, non-amino silicones and mixtures thereof.

The additional silicone(s) are other than the amino silicones of formula (I) as defined above.

This means that said additional silicones do not come under the definition of formula (I), for example because they do not have an amine number and/or molecular mass as defined for the silicones of formula (I) and/or because the definition of at least one of their radicals R, R′ and/or A is other than those indicated for the silicones of formula (I), or else because they have a different chemical structure to that of the silicones of formula (I).

The composition according to the invention may thus comprise one or more non-amino silicones, which may be solid or liquid (at 25° C., 1 atm), and volatile or nonvolatile. Preferably, the non-amino silicones are chosen from nonvolatile liquid silicones.

The non-amino silicones that may be used may be soluble or insoluble in the composition according to the invention; they may be in oil, wax, resin or gum form; silicone oils and gums are preferred.

Silicones are particularly described in detail in Walter Noll's “Chemistry and Technology of Silicones” (1968), Academic Press.

The volatile silicones may be chosen from those with a boiling point of between 60° C. and 260° C. (at atmospheric pressure, 1 atm) and in particular from:

i) cyclic polydialkylsiloxanes including from 3 to 7 and preferably 4 to 5 silicon atoms, such as
octamethylcyclotetrasiloxane (D4) and decamethylcyclopentasiloxane (D5). Mention may be made of the products sold under the name Volatile Silicone 7207 by Union Carbide or Silbione 70045 V 2 by Rhodia, Volatile Silicone 7158 by Union Carbide or Silbione 70045 V 5 by Rhodia;
cyclocopolymers of the dimethylsiloxane/methylalkylsiloxane type having the chemical structure:

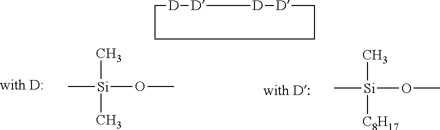

Mention may be made of Volatile Silicone FZ 3109 sold by Union Carbide.
mixtures of cyclic silicones with silicon-derived organic compounds, such as the mixture of octamethylcyclotetrasiloxane and of tetratrimethylsilylpentaerythritol (50/50) and the mixture of octamethylcyclotetrasiloxane and of 1,1′-oxy(2,2,2′,2′,3,3′-hexatrimethylsilyloxy)bisneopentane;

ii) linear polydialkylsiloxanes having 2 to 9 silicon atoms, which generally have a viscosity of less than or equal to 5×10.6 m²/s at 25° C., such as decamethyltetrasiloxane. Other silicones belonging to this category are described in the article published in Cosmetics and Toiletries, Vol. 91, January 76, pages 27-32—Todd & Byers Volatile silicone fluids for cosmetics; mention may be made of the product sold under the name SH 200 by Toray Silicone.

Among the nonvolatile silicones, mention may be made, alone or as a mixture, of polydialkylsiloxanes and particularly polydimethylsiloxanes (PDMS or dimethicone), polydiarylsiloxanes, polyalkylarylsiloxanes, silicone gums and resins, and also non-amino organopolysiloxanes (or organomodified polysiloxanes, or alternatively organomodified silicones) which are polysiloxanes including in their structure one or more non-amino organofunctional groups, generally attached via a hydrocarbon-based group, and preferably chosen from aryl groups, alkoxy groups and polyoxyethylene and/or polyoxypropylene groups.

The organomodified silicones may be polydiarylsiloxanes, particularly polydiphenylsiloxanes, and polyalkylarylsiloxanes functionalized with the organofunctional groups mentioned previously. The polyalkylarylsiloxanes are particularly chosen from linear and/or branched polydimethyl/methylphenylsiloxanes and polydimethyl/diphenylsiloxanes.

Among the organomodified silicones, mention may be made of organopolysiloxanes including:
polyoxyethylene and/or polyoxypropylene groups optionally including C₆-C₂₄ alkyl groups, such as dimethicone copolyols, and particularly those sold by the company Dow Corning under the name DC 1248 or the oils Silwet® L 722, L 7500, L 77 and L 711 from the company Union Carbide; or alternatively (C₁₂)alkylmethicone copolyols, and particularly those sold by the company Dow Corning under the name Q2-5200;
thiol groups, such as the products sold under the names GP 72 A and GP 71 from Genesee;
alkoxylated groups, such as the product sold under the name Silicone Copolymer F-755 by SWS Silicones and Abil Wax® 2428, 2434 and 2440 by the company Goldschmidt;
hydroxylated groups, such as polyorganosiloxanes bearing a hydroxyalkyl function;
acyloxyalkyl groups, such as the polyorganosiloxanes described in patent US-A-4 957 732;
anionic groups of the carboxylic acid type, as described, for example, in EP 186 507, or of the alkylcarboxylic type, such as the product X-22-3701E from the company Shin-Etsu; or else of the 2-hydroxyalkylsulfonate or 2-hydroxyalkylthiosulfate type, such as the products sold by the company Goldschmidt under the names Abil® S201 and Abil® S255;

The silicones can also be chosen from polydialkylsiloxanes, among which mention may be made mainly of polydimethylsiloxanes having trimethylsilyl end groups (CTFA: dimethicone). Among these polydialkylsiloxanes, mention may be made of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 with a viscosity of 60 000 mm²/s;
the Viscasil® oils from Momentive Performance Materials and certain oils of the SF series (SF 96, SF 18) from Momentive Performance Materials.

Mention may also be made of polydimethylsiloxanes having dimethylsilanol end groups (CTFA: dimethiconol), such as the oils of the 48 series from the company Rhodia.

In this category of polydialkylsiloxanes, mention may also be made of the products sold under the names Abil Wax® 9800 and 9801 by the company Goldschmidt, which are polydi(C₁-C₂₀)alkylsiloxanes.

Products that may be used more particularly in accordance with the invention are mixtures such as:
mixtures formed from a polydimethylsiloxane with a hydroxyl-terminated chain, or dimethiconol (CTFA), and from a cyclic polydimethylsiloxane, also known as cyclomethicone (CTFA), such as the product Q2-1401 sold by the company Dow Corning.

The polyalkylarylsiloxanes are particularly chosen from linear and/or branched polydimethyl/methylphenylsiloxanes and polydimethyl/diphenylsiloxanes with a viscosity ranging from 1×10⁻⁵ to 5×10⁻² m²/s at 25° C.

Among these polyalkylarylsiloxanes, mention may be made of the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
the silicones of the PN and PH series from Bayer, such as the products PN1000 and PH1000;
certain oils of the SF series from Momentive Performance Materials, such as SF 1023, SF 1154, SF 1250 and SF 1265.

The non-amino silicones that are more particularly preferred according to the invention are polydimethylsiloxanes containing trimethylsilyl end groups (CTFA: dimethicone).

The composition according to the invention may comprise one or more amino silicones other than the amino silicones of formula (I) as defined above,

The term “amino silicone” denotes any silicone including at least one primary, secondary or tertiary amine or a quaternary ammonium group.

The amino silicones that may be used according to the present invention may be volatile or nonvolatile and cyclic, linear or branched, and preferably have a viscosity ranging from 5×10⁻⁶ to 2.5 m²/s at 25° C., for example from 1×10⁻⁵ to 1 m²/s. Preferably, the additional amino silicones are chosen from nonvolatile liquid silicones (25° C., 1 atm).

Preferably, the amino silicone(s) are chosen, alone or as mixtures, from the following compounds:

A) the polysiloxanes corresponding to formula (Ia):

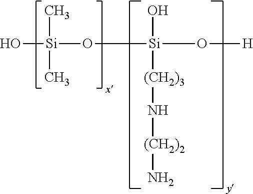

in which x′ and y′ are integers such that the weight-average molecular mass (Mw) is between 5000 and 500 000 g/mol;

B) the amino silicones corresponding to formula (IIa):

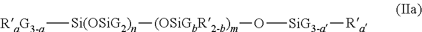

in which:
G, which is identical or different, denotes a hydrogen atom or a phenyl, OH, C₁-C₈ alkyl, for example methyl, or C₁-C₈ alkoxy, for example methoxy, group;
a and a′, which are identical or different, denote 0 or an integer from 1 to 3, in particular 0, with the proviso that at least one of a and a′ is equal to zero,
b denotes 0 or 1, in particular 1,
m and n are numbers such that the sum (n+m) ranges from 1 to 2000 and in particular from 50 to 150, it being possible for n to denote a number from 0 to 1999 and particularly from 49 to 149, and m to denote a number from 1 to 2000 and particularly from 1 to 10; and
R′, which is identical or different, denotes a monovalent radical of formula —C_{q}H_{2q}L in which q is a number ranging from 2 to 8 and L is an optionally quaternized amino group chosen from the following groups: —NR″-Q-N(R″)₂, —N(R″)₂, —N⁺(R″)₃A⁻, —N⁺H(R″)₂A⁻, —N⁺H₂(R″)A⁻, —NR″-Q-N⁺(R″)H₂A⁻, —NR″-Q-N⁺(R″)₂H A⁻ and —NR″-Q-N⁺(R″)₃A⁻, in which R″, which is identical or different, denotes hydrogen, phenyl, benzyl or a saturated monovalent hydrocarbon-based radical, for example a C₁-C₂₀ alkyl radical; Q denotes a linear or branched group of formula CᵣH₂ᵣ, r being an integer ranging from 2 to 6, preferably from 2 to 4; and A represents a cosmetically acceptable anion, particularly a halide, such as fluoride, chloride, bromide or iodide.

Preferably, the amino silicones of formula (IIa) may be chosen from:

(i) the “trimethylsilyl amodimethicone” silicones corresponding to formula (III):

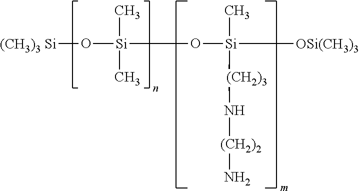

in which m and n are numbers such that the sum (n+m) ranges from 1 to 2000, preferably from 20 to 1000, in particular from 50 to 600, better still from 50 to 150; it being possible for n to denote a number from 0 to 1999 and particularly from 49 to 149 and it being possible for m to denote a number from 1 to 2000 and particularly from 1 to 10; (ii) the silicones of formula (IV) below:

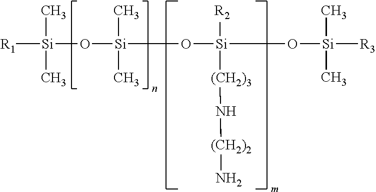

in which:
m and n are numbers such that the sum (n+m) ranges from 1 to 1000, in particular from 50 to 250 and more particularly from 100 to 200; n denoting a number from 0 to 999 and particularly from 49 to 249 and more particularly from 125 to 175, and m denoting a number from 1 to 1000, particularly from 1 to 10 and more particularly from 1 to 5; and
R₁, R₂ and R₃, which are identical or different, represent a hydroxyl or C₁-C₄ alkoxy radical, at least one of the radicals R₁ to R₃ denoting an alkoxy radical.

Preferably, the alkoxy radical is a methoxy radical.

The hydroxyl/alkoxy mole ratio preferably ranges from 0.2:1 to 0.4:1 and preferably from 0.25:1 to 0.35:1 and more particularly is equal to 0.3:1.

The weight-average molecular mass (Mw) of these silicones preferably ranges from 2000 to 1 000 000 g/mol and more particularly from 3500 to 200 000 g/mol;

(iii) the silicones of formula (V) below:

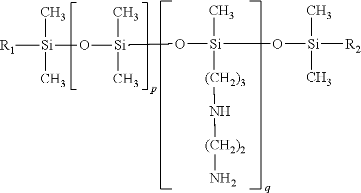

in which:
p and q are numbers such that the sum (p+q) ranges from 1 to 1000, in particular from 50 to 350 and more particularly from 150 to 250; p denoting a number from 0 to 999, particularly from 49 to 349 and more particularly from 159 to 239, and q denoting a number from 1 to 1000, particularly from 1 to 10 and more particularly from 1 to 5; and
R₁ and R₂, which are different, represent a hydroxyl or C₁-C₄ alkoxy radical, at least one of the radicals R₁ or R₂ denoting an alkoxy radical.

Preferably, the alkoxy radical is a methoxy radical.

The hydroxyl/alkoxy mole ratio generally ranges from 1:0.8 to 1:1.1 and preferably from 1:0.9 to 1:1 and more particularly is equal to 1:0.95.

The weight-average molecular mass (Mw) of the silicone preferably ranges from 2000 to 200 000 g/mol, more preferentially from 5000 to 100 000 g/mol and in particular from 10 000 to 50 000 g/mol.

The commercial products comprising silicones of structure (IV) or (V) may include in their composition one or more other amino silicones, the structure of which is different from formula (IV) or (V). A product containing amino silicones of structure (IV) is sold by the company Wacker under the name Belsil® ADM 652. A product containing amino silicones of structure (V) is sold by Wacker under the name Fluid WR 1300®. Another product containing amino silicones of structure (XIV) is sold by Wacker under the name Belsil ADM LOG 1®.

When these amino silicones are used, one particularly advantageous embodiment consists in using them in the form of an oil-in-water emulsion. The oil-in-water emulsion may comprise one or more surfactants. The surfactants may be of any nature but are preferably cationic and/or nonionic. The number-average size of the silicone particles in the emulsion generally ranges from 3 nm to 500 nm. Preferably, particularly as amino silicones of formula (V), use is made of microemulsions of which the mean particle size ranges from 5 nm to 60 nm (limits included) and more particularly from 10 nm to 50 nm (limits included). Thus, use may be made according to the invention of the amino silicone microemulsions of formula (V) sold under the names Finish CT 96 E® or SLM 28020® by the company Wacker;

(iv) the silicones of formula (VI) below:

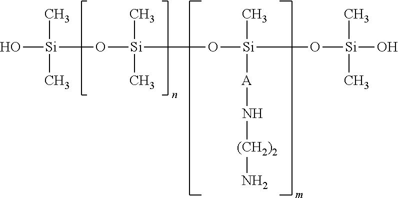

in which:
m and n are numbers such that the sum (n+m) ranges from 1 to 2000 and in particular from 50 to 150, n denoting a number from 0 to 1999 and particularly from 49 to 149, and m denoting a number from 1 to 2000 and particularly from 1 to 10; and
A denotes a linear or branched alkylene radical having from 4 to 8 carbon atoms and preferably 4 carbon atoms. This radical is preferably linear.

The weight-average molecular mass (Mw) of these amino silicones preferably ranges from 2000 to 1 000 000 g/mol and more particularly from 3500 to 200 000 g/mol.

A silicone corresponding to this formula is, for example, Xiameter MEM 8299 Emulsion from Dow Corning;

(v) the silicones of formula (VII) below:

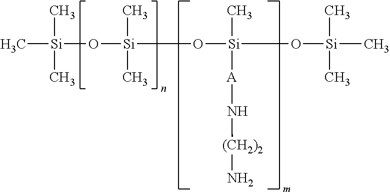

in which:
m and n are numbers such that the sum (n+m) ranges from 1 to 2000 and in particular from 50 to 150, it being possible for n to denote a number from 0 to 1999 and particularly from 49 to 149, and m to denote a number from 1 to 2000 and particularly from 1 to 10; and
A denotes a linear or branched alkylene radical having from 4 to 8 carbon atoms and preferably 4 carbon atoms. This radical is preferably branched.

The weight-average molecular mass (Mw) of these amino silicones preferably ranges from 500 to 1 000 000 g/mol and more particularly from 1000 to 200 000 g/mol.

A silicone corresponding to this formula is, for example, DC2-8566 Amino Fluid from Dow Corning;

C) the amino silicones corresponding to formula (VIII):

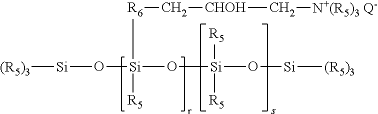

in which:
R₅ represents a monovalent hydrocarbon-based radical having from 1 to 18 carbon atoms, and in particular a C₁-C₁₈ alkyl or C₂-C₁₈ alkenyl radical, for example methyl;
R₆ represents a divalent hydrocarbon-based radical, particularly a C₁-C₁₈ alkylene radical or a divalent C₁-C₁₈, for example C₁-C₈, alkyleneoxy radical linked to the Si via an SiC bond;
Q⁻ is an anion such as a halide ion, particularly chloride, or an organic acid salt, particularly acetate;
r represents a mean statistical value ranging from 2 to 20 and in particular from 2 to 8;
   and
s represents a mean statistical value ranging from 20 to 200 and in particular from 20 to 50.

D) the quaternary ammonium silicones of formula (IX):

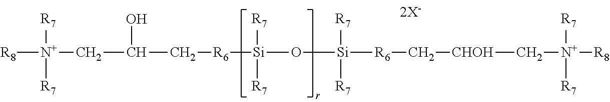

in which:
R_{7′} which are identical or different, represent a monovalent hydrocarbon-based radical having from 1 to 18 carbon atoms, and in particular a C₁-C₁₈ alkyl radical, a C₂-C₁₈ alkenyl radical or a ring comprising 5 or 6 carbon atoms, for example methyl;
R₆ represents a divalent hydrocarbon-based radical, particularly a C₁-C₁₈ alkylene radical or a divalent C₁-C₁₈, for example C₁-C₈, alkyleneoxy radical linked to the Si via an SiC bond;
R_{8′} which are identical or different, represent a hydrogen atom, a monovalent hydrocarbon-based radical having from 1 to 18 carbon atoms, and in particular a C₁-C₁₈ alkyl radical, a C₂-C₁₈ alkenyl radical or a —R₆—NHCOR₇ radical;
X⁻ is an anion such as a halide ion, particularly chloride, or an organic acid salt, particularly acetate; and
r represents a mean statistical value ranging from 2 to 200 and in particular from 5 to 100.

These silicones are for example described in patent application EP-A-0 530 974; mention may in particular be made of the silicone having the INCI name: Quaternium 80.

Silicones falling within this category are the silicones sold by the company Goldschmidt under the names Abil Quat 3270, Abil Quat 3272 and Abil Quat 3474;

E) the amino silicones of formula (X):

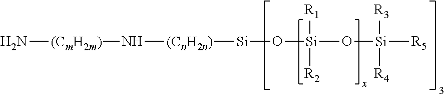

in which:
R₁, R₂, R₃ and R₄, which are identical or different, denote a C₁-C₄ alkyl radical or a phenyl group,
R₆ denotes a C₁-C₄ alkyl radical or a hydroxyl group,
n is an integer ranging from 1 to 5,
m is an integer ranging from 1 to 5, and
x is chosen such that the amine number ranges from 0.01 to 1 meq/g.

F) the multiblock polyoxyalkylenated amino silicones, of (AB)ₙ type, A being a polysiloxane block and B being a polyoxyalkylenated block including at least one amine group.

Said silicones are preferably constituted of repeating units having the following general formulae:

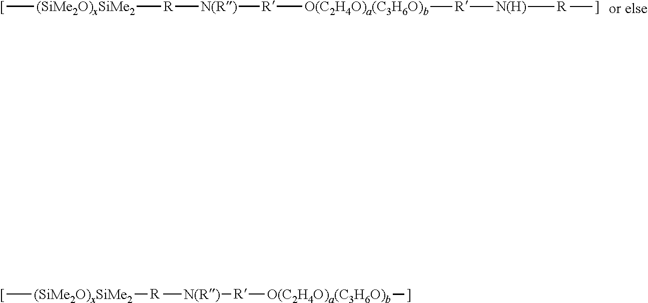

in which:
a is an integer greater than or equal to 1, preferably ranging from 5 to 200 and more particularly ranging from 10 to 100;
b is an integer between 0 and 200, preferably ranging from 4 to 100 and more particularly between 5 and 30;
x is an integer ranging from 1 to 10 000 and more particularly from 10 to 5000;
R″ is a hydrogen atom or a methyl;
R, which are identical or different, represent a linear or branched divalent C₂-C₁₂ hydrocarbon-based radical, optionally including one or more heteroatoms such as oxygen; preferably, R, which are identical or different, denote an ethylene radical, a linear or branched propylene radical, a linear or branched butylene radical or a CH₂CH₂CH₂OCH₂CH(OH)CH₂— radical; preferentially, R denote a CH₂CH₂CH₂OCH₂CH(OH)CH₂— radical; and
R′, which are identical or different, represent a linear or branched divalent C₂-C₁₂ hydrocarbon-based radical, optionally including one or more heteroatoms such as oxygen; preferably, R′, which are identical or different, denote an ethylene radical, a linear or branched propylene radical, a linear or branched butylene radical or a CH₂CH₂CH₂OCH₂CH(OH)CH₂— radical; preferentially, R′ denote —CH(CH₃)—CH₂—.

The siloxane blocks preferably represent between 50 mol % and 95 mol % of the total weight of the silicone, more particularly from 70 mol % to 85 mol %.

The amine content is preferably between 0.02 and 0.5 meq/g of copolymer in a 30% solution in dipropylene glycol, more particularly between 0.05 and 0.2.

The weight-average molecular mass (Mw) of the silicone is preferably between 5000 and 1 000 000 g/mol and more particularly between 10 000 and 200 000 g/mol.

Mention may particularly be made of the silicones sold under the name Silsoft A-843 or Silsoft A+ by Momentive.

G) the amino silicones of formulae (XI) and (XII):

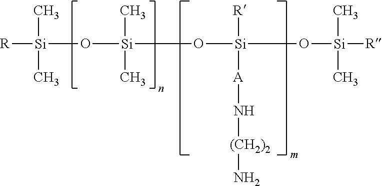

in which:
R, R′ and R″, which are identical or different, denote a C₁-C₄ alkyl group or a hydroxyl group,
A denotes a C₃ alkylene radical; and
m and n are numbers such that the weight-average molecular mass of the compound is between 5000 and 500 000;

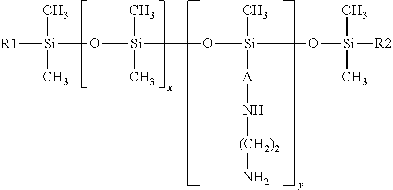

in which:
x and y are numbers ranging from 1 to 5000; preferably, x ranges from 10 to 2000 and more preferentially from 100 to 1000; preferably, y ranges from 1 to 100;
R₁ and R₂, which are identical or different, preferably identical, denote a linear or branched, saturated or unsaturated alkyl group comprising from 6 to 30 carbon atoms, preferably from 8 to 24 carbon atoms and more preferentially from 12 to 20 carbon atoms; and
A denotes a linear or branched alkylene radical having from 2 to 8 carbon atoms.

Preferably, A comprises from 3 to 6 carbon atoms, more preferentially 4 carbon atoms; preferably, A is branched. Mention may be made in particular of the following divalent groups: —CH₂CH₂CH₂— and —CH₂CH(CH₃)CH₂—.

Preferably, R₁ and R₂ are independent saturated linear alkyl groups comprising 6 to 30 carbon atoms, preferably 8 to 24 carbon atoms and in particular from 12 to 20 carbon atoms; mention may be made in particular of dodecyl, tetradecyl, pentadecyl, hexadecyl, heptadecyl, octadecyl, nonadecyl and eicosyl groups; and preferentially, R₁ and R₂, which are identical or different, are chosen from hexadecyl (cetyl) and octadecyl (stearyl) groups.

Preferably, in the silicone of formula (XII):
x ranges from 10 to 2000 and in particular from 100 to 1000;
y ranges from 1 to 100;
A comprises from 3 to 6 carbon atoms and particularly 4 carbon atoms; preferably, A is branched; more particularly, A is chosen from the following divalent groups: —CH₂CH₂CH₂ and —CH₂CH(CH₃)CH₂—; and
R₁ and R₂ independently are saturated linear alkyl groups comprising from 6 to 30 carbon atoms, preferably from 8 to 24 carbon atoms and in particular from 12 to 20 carbon atoms; particularly chosen from dodecyl, tetradecyl, pentadecyl, hexadecyl, heptadecyl, octadecyl, nonadecyl and eicosyl groups; preferentially, R₁ and R₂, which are identical or different, are chosen from hexadecyl (cetyl) and octadecyl (stearyl) groups.

A silicone of formula (XII) that is preferred is bis-cetearyl amodimethicone. Mention may be made in particular of the amino silicone sold under the name Silsoft AX by Momentive.

H) polysiloxanes and particularly polydimethylsiloxanes, including primary amine groups at only one chain end or on side chains, such as those of formula (XIV), (XV) or (XVI):

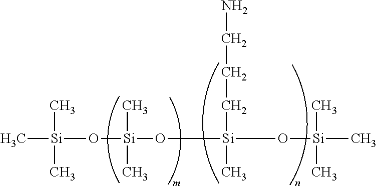

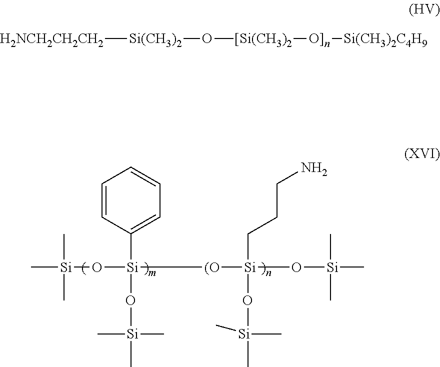

In formula (XIV), the values of n and m are such that the weight-average molecular mass of the amino silicone is between 1000 and 55 000.

As examples of amino silicones of formula (XIV), mention may be made of the products sold under the names AMS-132, AMS-152, AMS-162, AMS-163, AMS-191 and AMS-1203 by the company Gelest and KF-8015 by the company Shin-Etsu.

In formula (XV), the value of n is such that the weight-average molecular mass of the amino silicone is between 500 and 3000.

As examples of amino silicones of formula (XV), mention may be made of the products sold under the names MCR-A11 and MCR-A12 by the company Gelest.

In formula (XVI), the values of n and m are such that the weight-average molecular mass of the amino silicone is between 500 and 50 000.

As examples of amino silicones of formula (XVI), mention may be made of the aminopropyl phenyl trimethicone sold under the name DC 2-2078 Fluid by the company Dow Corning.

The composition according to the invention may also comprise, as silicone, an amino silicone corresponding to formula (XVIII) below:

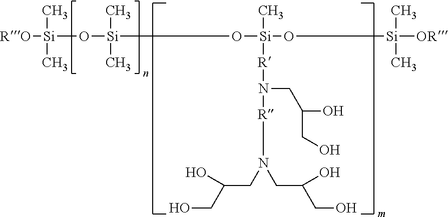

in which:
n is a number between 1 and 1000, preferably between 10 and 500, better still between 25 and 100, even better still between 50 and 80;
m is a number between 1 and 200, preferably between 1 and 100, better still between 1 and 10 and even better still between 1 and 5;
R′″, which are identical or different, preferably identical, are saturated or unsaturated, linear or branched, alkyl radicals comprising from 8 to 30 carbon atoms, preferably from 10 to 24 carbon atoms, particularly from 12 to 18 carbon atoms; it being possible for said radicals optionally to be substituted with one or more hydroxyl OH groups;
R′ is a linear or branched divalent alkylene radical having from 1 to 6 carbon atoms, particularly from 2 to 5 carbon atoms;
R″ is a linear or branched divalent alkylene radical having from 1 to 6 carbon atoms, particularly from 1 to 5 carbon atoms.

Preferably, the R′″, which are identical or different, are saturated linear alkyl radicals comprising from 8 to 30 carbon atoms, preferably from 10 to 24 carbon atoms, particularly from 12 to 18 carbon atoms; mention may be made in particular of dodecyl, C13, tetradecyl, pentadecyl, hexadecyl, heptadecyl and octadecyl radicals; preferentially, the R′″, which are identical or different, are chosen from saturated linear alkyl radicals having from 12 to 16 carbon atoms, particularly C13, C14 or C15 radicals, alone or as a mixture, and better still represent a mixture of C13, C14 and C15.

Preferably, the R′″ are identical.

Preferably, R′ is a linear or branched, preferably branched, divalent alkylene radical comprising from 1 to 6 carbon atoms, particularly from 2 to 5 carbon atoms; particularly a —CH₂—CH₂—CH₂—, —CH₂—CH(CH₃)—CH₂— or —CH₂—CH₂—CH(CH₃)— radical.

Preferably, R″ is a linear divalent alkylene radical comprising from 1 to 6 carbon atoms, particularly from 1 to 4 carbon atoms, in particular a —CH₂—CH₂— radical.

Preferentially, the composition may comprise one or more silicones of formula (XVIII) in which:
n is a number between 50 and 80;
m is a number between 1 and 5;
R′″, which are identical, are saturated linear alkyl radicals comprising from 12 to 18 carbon atoms;
R′ is a divalent alkylene radical having from 2 to 5 carbon atoms;
R″ is a linear divalent alkylene radical having from 1 to 4 carbon atoms.

Even better still, the composition may comprise one or more silicones of formula (XVIII) in which:
n is a number between 50 and 80;
m is a number between 1 and 5;
R′″, which are identical, are saturated linear alkyl radicals comprising from 13 to 15 carbon atoms;
R′ is a —(CH₂)₃—, —CH₂—CH(CH₃)—CH₂— or —CH₂—CH₂—CH(CH₃)— radical, and
R″ is a —(CH₂)₂— radical.

A most particularly preferred silicone of formula (XVIII) is Bis(C13-15 Alkoxy) PG-Amodimethicone (INCI name). Mention may particularly be made of the silicone sold under the name Dowsil 8500 Conditioning Agent by Dow.

Preferably, the additional silicone(s) may be chosen, alone or as a mixture, from nonvolatile liquid non-amino silicones, and particularly polydialkysiloxanes and more particularly polydimethylsiloxanes, in particular polydimethylsiloxanes having trimethylsilyl end groups; and/or nonvolatile liquid amino silicones, such as amodimethicones, aminopropyldimethicones and bis-aminopropyldimethicones, and also mixtures thereof.

In a preferred embodiment, the composition according to the invention does not comprise any additional silicones (0%).

When they are present, the composition according to the invention can comprise the additional silicone(s) in a total content preferably ranging from 0.01% to 2% by weight, better still ranging from 0.02% to 1% by weight and preferentially ranging from 0.05% to 0.5% by weight, relative to the total weight of the composition.

### Associative Polymers

The composition according to the invention may further comprise at least one associative polymer.

Preferably, the composition according to the invention comprises one or more associative polymers, preferably chosen from nonionic or cationic associative polymers.

For the purposes of the present invention, the term “polymer” means any compound derived from the polymerization by polycondensation or from the radical polymerization of monomers, at least one of which is other than an alkylene oxide, and of a monofunctional compound of formula RX, R denoting an optionally hydroxylated C₁₀-C₃₀ alkyl or alkenyl group, and X denoting a carboxylic acid, amine, amide, hydroxyl or ester group. All the compounds resulting solely from the simple condensation of an alkylene oxide with a fatty alcohol, a fatty ester, a fatty acid, a fatty amide or a fatty amine are in particular excluded.

For the purposes of the present invention, the term “associative polymer” means an amphiphilic polymer that is capable, in an aqueous medium, of reversibly associating with itself or with other molecules. It generally includes, in its chemical structure, at least one hydrophilic region or group and at least one hydrophobic region or group. The associative polymers according to the invention are polymers comprising at least one fatty chain including from 8 to 30 carbon atoms, the molecules of which are capable, in the formulation medium, of associating with each other or with molecules of other compounds. Preferably, the fatty chain includes from 10 to 30 carbon atoms.

A particular case of associative polymers is amphiphilic polymers, that is to say polymers including one or more hydrophilic parts or groups which make them water-soluble and one or more hydrophobic regions or groups (including at least one fatty chain) via which the polymers interact and assemble with each other or with other molecules.

The term “hydrophobic group” means a group or a polymer containing a saturated or unsaturated, linear or branched hydrocarbon-based chain, which may contain one or more heteroatoms such as P, O, N or S, or a radical containing a perfluoro or silicone chain. When it denotes a hydrocarbon-based group, the hydrophobic group includes at least 10 carbon atoms, preferably from 10 to 30 carbon atoms, in particular from 12 to 30 carbon atoms and preferentially from 18 to 30 carbon atoms. Preferentially, the hydrocarbon-based hydrophobic group originates from a monofunctional compound. By way of example, the hydrophobic group may be derived from a fatty alcohol, such as stearyl alcohol, dodecyl alcohol or decyl alcohol, or else from a polyalkylenated fatty alcohol, such as Steareth-100. It may also denote a hydrocarbon-based polymer, for instance polybutadiene.

For the purposes of the present invention, the term “fatty chain” means a linear or branched alkyl or alkenyl chain including at least 8 carbon atoms, preferably from 8 to 30 carbon atoms and better still from 10 to 22 carbon atoms.

For the purposes of the present invention, the term “fatty”, for instance a fatty alcohol, a fatty acid or a fatty amide, means a compound comprising, in its main chain, at least one saturated or unsaturated hydrocarbon-based chain, such as an alkyl or alkenyl chain, including at least 8 carbon atoms, preferably from 8 to 30 carbon atoms and better still from 10 to 22 carbon atoms.

Among the the anionic associative polymers, mention may particularly be made of:
(A) those including at least one hydrophilic unit and at least one fatty-chain allyl ether unit; and more particularly those:
   of which the hydrophilic unit is constituted by an ethylenic unsaturated anionic monomer, even more particularly by a vinylcarboxylic acid and most particularly by an acrylic acid or a methacrylic acid or mixtures thereof; and
   of which the fatty-chain allyl ether unit corresponds to the monomer of formula (I′) below:

CH₂═C(R′)—CH₂OBₙR

in which R′ denotes H or CH₃, B denotes an ethyleneoxy radical, n ranges from 0 to 100 and R denotes a hydrocarbon-based radical chosen from the alkyl, arylalkyl, aryl, alkylaryl and cycloalkyl radicals, comprising from 8 to 30 carbon atoms, preferably from 10 to 24 carbon atoms and even more particularly from 12 to 18 carbon atoms. Preferably, R′ denotes H, n=10 and R denotes a stearyl (C₁₈) radical.

Among these anionic associative polymers, preference is particularly given to polymers formed from 20% to 60% by weight of acrylic acid and/or of methacrylic acid, from 5% to 60% by weight of C₁-C₄ alkyl (meth)acrylates, from 2% to 50% by weight of fatty-chain allyl ether of formula (I′) and from 0% to 1% by weight of a crosslinking agent which is preferably a copolymerizable polyethylenic unsaturated monomer, such as diallyl phthalate, allyl (meth)acrylate, divinylbenzene, (poly)ethylene glycol dimethacrylate or methylenebisacrylamide.

Preference is most particularly given to crosslinked terpolymers of methacrylic acid, of ethyl acrylate and of polyethylene glycol (10 EO) stearyl alcohol ether (Steareth-10), particularly the product sold by the company BASF under the name Salcare SC80, which is a 30% aqueous emulsion of a crosslinked terpolymer of methacrylic acid, of ethyl acrylate and of steareth-10 allyl ether (40/50/10) having the INCI name Steareth-10 Allyl Ether/Acrylates Copolymer;
(B) those including at least one hydrophilic unit of unsaturated olefinic carboxylic acid type and at least one hydrophobic unit of (C₁₀-C₃₀)alkyl ester of unsaturated carboxylic acid type.

These polymers are preferably chosen from those for which the hydrophilic unit of unsaturated olefinic carboxylic acid type corresponds to the monomer of formula (II) below:

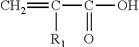

in which R₁ denotes H or CH₃ or C₂H₅, and for which the hydrophobic unit of (C₁₀-C₃₀)alkyl ester of unsaturated carboxylic acid type corresponds to the monomer of formula (III) below:

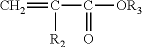

in which R₂ denotes H, CH₃ or C₂H₅ and R₃ denotes a C₁₀-C₃₀ and preferably C₁₂-C₂₂ alkyl radical.

(C₁₀-C₃₀) Alkyl esters of unsaturated carboxylic acids in accordance with the invention comprise, for example, lauryl acrylate, stearyl acrylate, decyl acrylate, isodecyl acrylate and dodecyl acrylate, and the corresponding methacrylates, lauryl methacrylate, stearyl methacrylate, decyl methacrylate, isodecyl methacrylate and dodecyl methacrylate.

Among these anionic associative polymers, use will more particularly be made of polymers formed from a mixture of monomers comprising:
(i) (meth)acrylic acid,
(ii) an ester of formula (III) described above in which R₂ denotes H or CH₃, R₃ denotes an alkyl radical having 12 to 22 carbon atoms, and optionally
(iii) and a crosslinking agent, which is a well-known copolymerizable polyethylenic unsaturated monomer, for instance diallyl phthalate, allyl (meth)acrylate, divinylbenzene, (poly)ethylene glycol dimethacrylate or methylenebisacrylamide.

Among anionic associative polymers of this type, preference is more particularly given to those constituted of from 95% to 60% by weight of (meth)acrylic acid, 4% to 40% by weight of C₁₀-C₃₀ alkyl acrylate and 0% to 6% by weight of crosslinking polymerizable monomer, or else to those constituted of from 98% to 96% by weight of (meth)acrylic acid, 1% to 4% by weight of C₁₀-C₃₀ alkyl acrylate and 0.1% to 0.6% by weight of crosslinking polymerizable monomer, such as those described previously.

Mention may in particular be made of the products sold by the company Lubrizol under the trade names Pemulen TR1, Pemulen TR2, Carbopol 1382, Carbopol ETD 2020, Carbopol Ultrez 20, Carbopol Ultrez 21, having the INCI name Acrylates/C10-30 Alkyl Acrylate Crosspolymer, and even more preferentially Pemulen TR1 and Carbopol 1382;
(C) maleic anhydride/C₃₀-C₃₈ α-olefin/alkyl maleate terpolymers, such as the maleic anhydride/C₃₀-C₃₈ α-olefin/isopropyl maleate copolymer, in particular the product sold under the name Performa V 1608 by the company Newphase Technologies (INCI name: C30-38 Olefin/Isopropyl Maleate/MA Copolymer);
(D) acrylic terpolymers comprising
   (a) 20% to 70% by weight of an α,β-monoethylenically unsaturated carboxylic acid,
   (b) 20% to 80% by weight of a non-surfactant α,β-monoethylenically unsaturated monomer other than (a), and
   (c) 0.5% to 60% by weight of a nonionic monourethane which is the reaction product of a monohydric surfactant with a monoethylenically unsaturated monoisocyanate.

Mention may particularly be made of the methacrylic acid/methyl acrylate/dimethyl(meta-isopropenyl)benzyl isocyanate of ethoxylated (40 EO) behenyl alcohol terpolymer, particularly as a 25% aqueous dispersion, such as the product Viscophobe DB1000 sold by the company Amerchol (Dow Chemical), having the INCI name Polyacrylate-3.
(E) copolymers including, among their monomers
   (i) an α,β-monoethylenically unsaturated carboxylic acid, such as acrylic acid or methacrylic acid, and
   (ii) an ester of α,β-monoethylenically unsaturated carboxylic acid, particularly acrylic or methacrylic acid, and of fatty alcohol, particularly C8-C32 oxyalkylenated fatty alcohol, particularly comprising 2 to 100 mol of ethylene oxide, in particular 4 to 50, or even 10 to 40 EO.

Mention may in particular made, as monomers, of behenyl or stearyl (meth)acrylate comprising 10 to 40 EO, in particular 18 to 30 EO.

Preferentially, these compounds also comprise, as monomer, an ester of an α,β-monoethylenically unsaturated carboxylic acid and of a C₁-C₄ alcohol, particularly a C₁-C₄ alkyl (meth)acrylate.

Preferably, these copolymers comprise at least one (meth)acrylic acid monomer, at least one C₁-C₄ alkyl (meth)acrylate monomer and at least one C₈-C₃₂ alkyl (meth)acrylate monomer which is oxyethylenated, comprising from 2 to 100 mol EO, in particular from 4 to 50 EO, or even from 10 to 40 EO.

By way of example, mention may be made of Aculyn 22 sold by the company Rohm and Haas, which is an methacrylic acid/ethyl acrylate/oxyalkylenated stearyl methacrylate terpolymer (INCI name: Acrylates/Steareth-20 Methacrylate Copolymer), or also of Aculyn 28 sold by Rohm and Haas, which is an methacrylic acid/ethyl acrylate/oxyalkylenated behenyl methacrylate terpolymer (INCI name: Acrylates/Beheneth-25 Methacrylate Copolymer), and also of the Novethix L-10 Polymer sold by Lubrizol;
(F) associative polymers including at least one ethylenically unsaturated monomer bearing a sulfonic group, in free or partially or totally neutralized form and comprising at least one hydrophobic portion.

Among the polymers of this type, mention may be made more especially of:
crosslinked or non-crosslinked, neutralized or non-neutralized copolymers including from 15% to 60% by weight of AMPS (2-acrylamido-2-methylpropanesulfonic acid or salt) units and from 40% to 85% by weight of (C₈-C₁₆)alkyl (meth)acrylate units relative to the polymer, such as those described in patent application EP-A-750 899;
terpolymers comprising from 10 mol % to 90 mol % of acrylamide units, from 0.1 mol % to 10 mol % of AMPS units and from 5 mol % to 80 mol % of n-(C₆-C₈)alkylacrylamide units, such as those described in U.S. Pat. No. 5,089,578;
copolymers of totally neutralized AMPS and of dodecyl methacrylate, and also copolymers of AMPS and of n-dodecylmethacrylamide, which are non-crosslinked and crosslinked;
copolymers constituted of AMPS units and of steareth-25 methacrylate units, such as Aristoflex HMS sold by the company Clariant (INCI name: Ammonium Acryloyldimethyltaurate/Steareth-25 Methacrylate Crosspolymer), or beheneth-25 methacrylate units, such as Aristoflex® HMB (INCI name: Ammonium Acryloyldimethyltaurate/Beheneth-25 Methacrylate Crosspolymer) sold by the company Clariant, or also steareth-8 methacrylate units, such as Aristoflex SNC® from Clariant (INCI name: Ammonium Acryloyldimethyltaurate/Steareth-8 Methacrylate Copolymer).
(G) associative polymers including at least one vinyllactam monomer and at least one α,β-monoethylenically unsaturated carboxylic acid monomer, such as terpolymers of vinylpyrrolidone, of acrylic acid and of C₁-C₂₀ alkyl methacrylate, for example lauryl methacrylate, such as the product sold by the company ISP under the name Acrylidone® LM (INCI name: VP/Acrylates/Lauryl Methacrylate Copolymer).

Among the cationic associative polymers, mention may be made of:
(A′) cationic associative polyurethanes, which may be represented by the general formula (Ia) below:

R—X—(P)ₙ-[L-(Y)ₘ]ᵣ-L′-(P′)ₚ—X′—R′

in which:
R and R′, which are identical or different, represent a hydrophobic group or a hydrogen atom;
X and X′, which are identical or different, represent a group including an amine function optionally bearing a hydrophobic group, or alternatively a group L″;
L, L′ and L″, which are identical or different, represent a group derived from a diisocyanate;
P and P′, which are identical or different, represent a group including an amine function optionally bearing a hydrophobic group;
Y represents a hydrophilic group;
r is an integer between 1 and 100 inclusive, preferably between 1 and 50 inclusive and in particular between 1 and 25 inclusive;
n, m and p are each, independently of each other, between 0 and 1000 inclusive; the molecule containing at least one protonated or quaternized amine function and at least one hydrophobic group.

Preferably, the only hydrophobic groups are the groups R and R′ at the chain ends.

One preferred family of cationic associative polyurethanes is the one corresponding to formula (Ia) described above, in which:
R and R′ both independently represent a hydrophobic group,
X and X′ each represent a group L″,
n and p are integers that are between 1 and 1000 inclusive, and
L, L′, L″, P, P′, Y and m have the meanings given above.

Another preferred family of cationic associative polyurethanes is the one corresponding to formula (Ia) above in which:
n=p=0 (the polymers do not include any units derived from a monomer containing an amine function, incorporated into the polymer during the polycondensation), the protonated amine functions result from the hydrolysis of excess isocyanate functions, at the chain end, followed by alkylation of the primary amine functions formed with alkylating agents containing a hydrophobic group, i.e. compounds of the type RQ or R′Q, in which R and R′ are as defined above and Q denotes a leaving group such as a halide or a sulfate, etc.

Yet another preferred family of cationic associative polyurethanes is the one corresponding to formula (Ia) above in which:
R and R′ both independently represent a hydrophobic group,
X and X′ both independently represent a group including a quaternary amine,
n=p=0, and
L, L′, Y and m have the meaning indicated above.

The number-average molecular mass (Mn) of the cationic associative polyurethanes is preferably between 400 and 500 000 inclusive, in particular between 1000 and 400 000 inclusive and ideally between 1000 and 300 000 inclusive.

Preferentially, the hydrocarbon-based group originates from a monofunctional compound.

By way of example, the hydrophobic group may be derived from a fatty alcohol such as stearyl alcohol, dodecyl alcohol or decyl alcohol. It may also denote a hydrocarbon-based polymer, for instance polybutadiene.

When X and/or X′ denote(s) a group including a tertiary or quaternary amine, X and/or X′ may represent one of the following formulae:

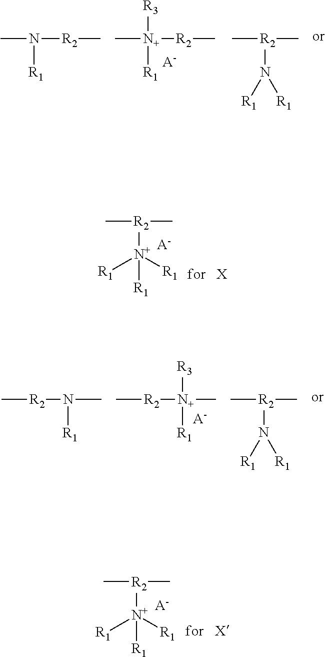

in which:
R₂ represents a linear or branched alkylene radical having from 1 to 20 carbon atoms, optionally including a saturated or unsaturated ring, or an arylene radical, it being possible for one or more of the carbon atoms to be replaced with a heteroatom chosen from N, S, O and P;
R₁ and R₃, which are identical or different, denote a linear or branched C₁-C₃₀ alkyl or alkenyl radical or an aryl radical, it being possible for at least one of the carbon atoms to be replaced with a heteroatom chosen from N, S, O and P;
A⁻ is a physiologically acceptable anionic counterion, such as a halide, for instance a chloride or bromide, or a mesylate.

The groups L, L′ and L″ represent a group of formula:

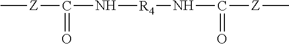

in which:
Z represents —O—, —S— or —NH—; and
R₄ represents a linear or branched alkylene radical having from 1 to 20 carbon atoms, optionally including a saturated or unsaturated ring, or an arylene radical, it being possible for one or more of the carbon atoms to be replaced with a heteroatom chosen from N, S, O and P.

The groups P and P′ comprising an amine function may represent at least one of the following formulae:

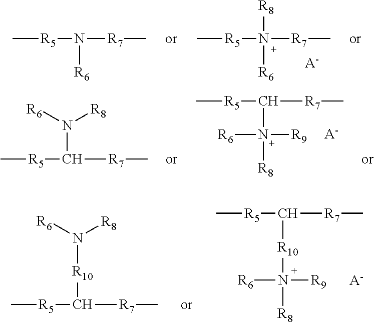

in which:
R₅ and R₇ have the same meanings as R_{z} defined above;
R₆, R₈ and R₉ have the same meanings as R₁ and R₃ defined above;
R₁₀ represents a linear or branched, optionally unsaturated alkylene group which may contain one or more heteroatoms chosen from N, O, S and P;
and A⁻ is a physiologically acceptable anionic counterion, such as a halide, for instance chloride or bromide, or mesylate.

As regards the meaning of Y, the term “hydrophilic group” means a polymeric or nonpolymeric water-soluble group.

By way of example, when it is not a polymer, mention may be made of ethylene glycol, diethylene glycol and propylene glycol.

When it is a hydrophilic polymer, mention may be made, for example, of polyethers, sulfonated polyesters and sulfonated polyamides, or a mixture of these polymers. The hydrophilic compound is preferentially a polyether and particularly a poly(ethylene oxide) or poly(propylene oxide).

The cationic associative polyurethanes of formula (Ia) according to the invention are formed from diisocyanates and from various compounds bearing functions containing labile hydrogen. The functions containing labile hydrogen may be alcohol, primary or secondary amine or thiol functions, giving, after reaction with the diisocyanate functions, polyurethanes, polyureas and polythioureas, respectively. In the present invention, the term “polyurethanes” encompasses these three types of polymer, namely polyurethanes per se, polyureas and polythioureas, and also copolymers thereof.

A first type of compound involved in the preparation of the polyurethane of formula (Ia) is a compound including at least one unit bearing an amine function. This compound may be multifunctional, but the compound is preferentially difunctional, that is to say that, according to a preferential embodiment, this compound includes two labile hydrogen atoms borne, for example, by a hydroxyl, primary amine, secondary amine or thiol function. A mixture of multifunctional and difunctional compounds in which the percentage of multifunctional compounds is low may also be used.

As mentioned above, this compound may include more than one unit containing an amine function. In this case, it is a polymer bearing a repetition of the unit containing an amine function.

Compounds of this type may be represented by one of the following formulae: HZ—(P)ₙ—ZH or HZ—(P′)ₚ—ZH, in which Z, P, P′, n and p are as defined above.

Examples that may be mentioned include N-methyldiethanolamine, N-tert-butyldiethanolamine and N-sulfoethyldiethanolamine.

The second compound involved in the preparation of the polyurethane of formula (Ia) is a diisocyanate corresponding to the formula: O═C═N—R₄—N═C═O in which R₄ is as defined above.

By way of example, mention may be made of methylenediphenyl diisocyanate, methylenecyclohexane diisocyanate, isophorone diisocyanate, toluene diisocyanate, naphthalene diisocyanate, butane diisocyanate and hexane diisocyanate.

A third compound involved in the preparation of the polyurethane of formula (Ia) is a hydrophobic compound intended to form the hydrophobic end groups of the polymer of formula (Ia).

This compound is constituted of a hydrophobic group and a function containing labile hydrogen, for example a hydroxyl, primary or secondary amine, or thiol function.

By way of example, this compound may be a fatty alcohol such as stearyl alcohol, dodecyl alcohol or decyl alcohol. When this compound includes a polymeric chain, it may be, for example, α-hydroxylated hydrogenated polybutadiene.

The hydrophobic group of the polyurethane of formula (Ia) may also result from the quaternization reaction of the tertiary amine of the compound including at least one tertiary amine unit. Thus, the hydrophobic group is introduced via the quaternizing agent. This quaternizing agent is a compound of the type RQ or R′Q, in which R and R′ are as defined above and Q denotes a leaving group such as a halide, a sulfate, etc.

The cationic associative polyurethane may also comprise a hydrophilic block. This block is provided by a fourth type of compound involved in the preparation of the polymer.

This compound may be multifunctional. It is preferably difunctional. It is also possible to have a mixture in which the percentage of multifunctional compound is low.

The functions containing labile hydrogen are alcohol, primary or secondary amine or thiol functions. This compound may be a polymer terminated at the chain ends with one of these functions containing labile hydrogen.

By way of example, when it is not a polymer, mention may be made of ethylene glycol, diethylene glycol and propylene glycol.

When it is a hydrophilic polymer, mention may be made, for example, of polyethers, sulfonated polyesters and sulfonated polyamides, or a mixture of these polymers. The hydrophilic compound is preferentially a polyether and particularly a poly(ethylene oxide) or poly(propylene oxide).

The hydrophilic group termed Y in formula (Ia) is optional. Specifically, the units containing a quaternary or protonated amine function may be sufficient to provide the solubility or water-dispersibility required for this type of polymer in an aqueous solution.

Although the presence of a hydrophilic group Y is optional, cationic associative polyurethanes including such a group are, however, preferred.
(B′) quaternized cellulose derivatives, and in particular:
i) quaternized celluloses modified with groups including at least one fatty chain, such as linear or branched alkyl, linear or branched arylalkyl or linear or branched alkylaryl groups including at least 8 carbon atoms, or mixtures thereof;
ii) quaternized hydroxyethylcelluloses modified with groups including at least one fatty chain, such as linear or branched alkyl, linear or branched arylalkyl or linear or branched alkylaryl groups including at least 8 carbon atoms, or mixtures thereof;
iii) the hydroxyethylcelluloses of formula (Ib):

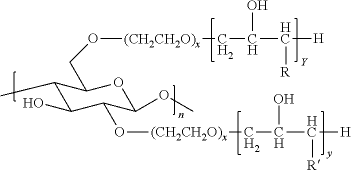

in which:
R and R′, which are identical or different, represent an ammonium group —RₐR_{b}R_{c}N⁺ Q⁻ in which Rₐ, R_{b} and R_{c}, which are identical or different, represent a hydrogen atom or a linear or branched C₁-C₃₀, preferentially C₁-C₂₀, alkyl group, such as methyl or dodecyl; and Q⁻ represents an anionic counterion, such as a halide, for instance a chloride or bromide; and
   n, x and y, which are identical or different, represent an integer between 1 and 10 000.

The alkyl radicals borne by the above quaternized celluloses i) or hydroxyethylcelluloses ii) preferably include from 8 to 30 carbon atoms. The aryl radicals preferably denote phenyl, benzyl, naphthyl or anthryl groups.

Mention may also be made, as examples of quaternized alkylhydroxyethylcelluloses bearing C₈ to C₃₀ fatty chains, of the product Quatrisoft LM 200® sold by the company Amerchol/Dow Chemical (INCI name: Polyquaternium-24) and the products Crodacel QM® (INCI name: PG-Hydroxyethylcellulose cocodimonium chloride), Crodacel QL® (C₁₂ alkyl) (INCI name: PG-Hydroxyethylcellulose lauryldimonium chloride) and Crodacel QS® (C₁₈ alkyl) (INCI name: PG-Hydroxyethylcellulose stearyldimonium chloride) sold by the company Croda.

Mention may also be made of the hydroxyethylcelluloses of formula (Ib) in which R represents a trimethylammonium halide and R′ represents a dimethyldodecylammonium halide; more preferentially, R represents trimethylammonium chloride —(CH₃)₃N⁺Cl⁻ and R′ represents dimethyldodecylammonium chloride —(CH₃)₂(C₁₂H₂₅)N⁺Cl⁻. This type of polymer is known under the trade name Softcat Polymer SL®, such as SL-100, SL-60, SL-30 and SL-5, from the company Amerchol/Dow Chemical, having the INCI name Polyquaternium-67.

More particularly, the polymers of formula (Ib) are those for which the viscosity is between 2000 and 3000 cPs inclusive. Preferentially, the viscosity is between 2700 and 2800 cPs inclusive. Typically, Softcat Polymer SL-5 has a viscosity of 2500 cPs, Softcat Polymer SL-30 has a viscosity of 2700 cPs, Softcat Polymer SL-60 has a viscosity of 2700 cPs and Softcat Polymer SL-100 has a viscosity of 2800 cPs.
(C′) cationic polyvinyllactams, particularly those comprising:
a) at least one monomer of vinyllactam or alkylvinyllactam type;
b) at least one monomer of structure (Ic) or (IIc) below:

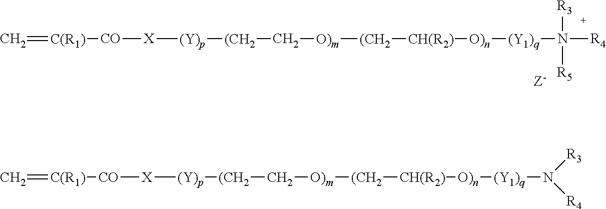

in which:
X denotes an oxygen atom or an NR6 radical,
R₁ and R₆ denote, independently of each other, a hydrogen atom or a linear or branched C₁-C₆ alkyl radical,
R₂ denotes a linear or branched C₁-C₄ alkyl radical,
   R₃, R₄ and R₅ denote, independently of each other, a hydrogen atom, a linear or branched C₁-C₃₀ alkyl radical or a radical of formula (IIIc):

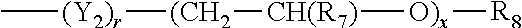

in which:
Y, Y₁ and Y₂ denote, independently of each other, a linear or branched C₂-C₁₆ alkylene radical,
R₇ denotes a hydrogen atom or a linear or branched C₁-C₄ alkyl radical or a linear or branched C₁-C₄ hydroxyalkyl radical,
R₈ denotes a hydrogen atom or a linear or branched C₁-C₃₀ alkyl radical,
p, q and r denote, independently of each other, either the value zero or the value 1,
m and n denote, independently of each other, an integer ranging from 0 to 100 inclusive,
x denotes an integer ranging from 1 to 100 inclusive,
Z denotes an anionic counterion of an organic or mineral acid, such as a halide, for instance chloride or bromide, or mesylate;
with the proviso that:
   at least one of the substituents R₃, R₄, R₅ or R₈ denotes a linear or branched C₉-C₃₀ alkyl radical,
   if m or n is other than zero, then q is equal to 1,
   if m or n is equal to zero, then p or q is equal to 0.

The cationic poly(vinyllactam) polymers according to the invention may be crosslinked or non-crosslinked and may also be block polymers.

Preferably, the counterion Z of the monomers of formula (Ic) is chosen from halide ions, phosphate ions, the methosulfate ion and the tosylate ion.

Preferably, R₃, R₄ and R₆ denote, independently of each other, a hydrogen atom or a linear or branched C₁-C₃₀ alkyl radical.

More preferentially, the monomer b) is a monomer of formula (Ic) for which, preferentially, m and n are equal to 0.

The vinyllactam or alkylvinyllactam monomer is preferably a compound of structure (IVc):

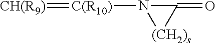

in which s denotes an integer ranging from 3 to 6; R₉ denotes a hydrogen atom or a linear or branched C₁-C₆ alkyl radical and R₁₀ denotes a hydrogen atom or a linear or branched C₁-C₅ alkyl radical, with the proviso that one at least of the radicals R₉ and R₁₀ denotes a hydrogen atom.

Even more preferentially, the monomer (IVc) is vinylpyrrolidone.

The cationic poly(vinyllactam) polymers according to the invention may also contain one or more additional monomers, preferably cationic or nonionic monomers.

As compounds that are particularly preferred, mention may be made of the following terpolymers comprising at least:
a) one monomer of formula (IVc),
b) one monomer of formula (Ic) in which p=1, q=0, R₃ and R₄ denote, independently of each other, a hydrogen atom or a C₁-C₅ alkyl radical and R₅ denotes a linear or branched C₉-C₂₄ alkyl radical, and
c) one monomer of formula (IIc) in which R₃ and R₄ denote, independently of each other, a hydrogen atom or a linear or branched C₁-C₅ alkyl radical.

Even more preferentially, use will be made of terpolymers comprising, by weight:
40% to 95% of monomer (a),
0.1% to 55% of monomer (c), and
0.25% to 50% of monomer (b).

Such polymers are particularly described in patent application WO-00/68282.

As cationic poly(vinyllactam) polymers according to the invention, use is in particular made of:
vinylpyrrolidone/dimethylaminopropylmethacrylamide/dodecyldimethylmethacrylamidopropylammonium tosylate terpolymers,
vinylpyrrolidone/dimethylaminopropylmethacrylamide/cocoyldimethylmethacrylamidopropylammonium tosylate terpolymers,
vinylpyrrolidone/dimethylaminopropylmethacrylamide/lauryldimethylmethacrylamidopropylammonium tosylate or chloride terpolymers.

The vinylpyrrolidone/dimethylaminopropylmethacrylamide/lauryldimethylmethylacrylamidopropylammonium chloride terpolymer is particularly sold by the company ISP under the names Styleze W10® and Styleze W20L® (INCI name: Polyquaternium-55).

The weight-average molecular mass (Mw) of the cationic poly(vinyllactam) polymers is preferably between 500 and 20 000 000, more particularly between 200 000 and 2 000 000 and preferentially between 400 000 and 800 000.
(D′) the cationic polymers obtained by polymerization of a mixture of monomers comprising one or more vinyl monomers substituted with one or more amino groups, one or more hydrophobic nonionic vinyl monomers and one or more associative vinyl monomers, such as described in patent application WO 2004/024779.

Among these polymers, mention will more particularly be made of the products from the polymerization of a monomer mixture comprising:
a di(C₁-C₄ alkyl)amino(C₁-C₆ alkyl) methacrylate,
one or more C₁-C₃₀ alkyl esters of (meth)acrylic acid,
a polyethoxylated C₁₀-C₃₀ alkyl methacrylate (20-25 mol of ethylene oxide units),
a 30/5 polyethylene glycol/polypropylene glycol allyl ether,
a hydroxy(C₂-C₆ alkyl) methacrylate, and
an ethylene glycol dimethacrylate.

Such a polymer is, for example, the compound sold by the company Lubrizol under the name Carbopol Aqua CC® and which corresponds to the INCI name Polyacrylate-1 Crosspolymer.

The nonionic associative polymers are preferably chosen, alone or as a mixture, from: (1) celluloses modified with groups including at least one fatty chain, particularly C₈-C₃₂ and better still C₁₄-C₂₈ alkyl; preferably from:
hydroxyethylcelluloses modified with groups including at least one fatty chain, particularly C₈-C₃₂ and better still C₁₄-C₂₈ alkyl, such as alkyl, arylalkyl or alkylaryl groups, or mixtures thereof, and in which the alkyl groups are preferably C₈-C₂₂, for instance the cetylhydroxyethylcellulose sold particularly under the reference Natrosol Plus Grade 330 CS (C₁₆ alkyls) sold by the company Ashland, or the product Polysurf 67CS sold by the company Ashland,
hydroxyethylcelluloses modified with polyalkylene glycol alkylphenol ether groups, such as the product Amercell Polymer HM-1500 (polyethylene glycol (15) nonylphenol ether) sold by the company Amerchol,
and mixtures thereof.

(2) hydroxypropyl guars modified with groups including at least one fatty chain, particularly C₈-C₃₂ and better still C₁₄-C₂₈ alkyl, such as the product Esaflor HM 22 (C₂₂ alkyl chain) sold by the company Lamberti, and the products RE210-18 (C₁₄ alkyl chain) and RE205-1 (C₂₀ alkyl chain) sold by the company Rhodia.

(3) copolymers of vinylpyrrolidone and of fatty-chain hydrophobic monomers, particularly C₈-C₃₂ and better still C₁₄-C₂₉ alkyl. Examples that may be mentioned include:
the vinylpyrrolidone/hexadecene copolymer and particularly the products Antaron V216 or Ganex V216 sold by the company ISP;
the vinylpyrrolidone/eicosene copolymer and particularly the products Antaron V220 or Ganex V220 sold by the company ISP.

(4) copolymers of C₁-C₆ alkyl methacrylates or acrylates and of amphiphilic monomers including at least one fatty chain, particularly C₈-C₃₂ and better still C_{W}-C₂₈ alkyl, for instance the methyl acrylate/stearyl acrylate oxyethylenated copolymer sold by the company Goldschmidt under the name Antil 208.

(5) copolymers of hydrophilic methacrylates or acrylates and of hydrophobic monomers including at least one fatty chain, particularly C₈-C₃₂ and better still C₁₄-C₂₉ alkyl, for instance the polyethylene glycol methacrylate/lauryl methacrylate copolymer.

(6) polyurethane polyethers including in their chain both hydrophilic blocks usually of polyoxyethylenated nature and hydrophobic blocks, which may be aliphatic sequences alone and/or cycloaliphatic and/or aromatic sequences.

(7) polymers comprising an aminoplast ether backbone having at least one fatty chain, particularly C₈-C₃₂ and better still C₁₄-C₂₈ alkyl, such as the Pure Thix compounds sold by the company Sud-Chemie.

Preferably, the polyurethane polyethers include at least two hydrocarbon-based lipophilic chains having from 8 to 30 carbon atoms, separated by a hydrophilic block, it being possible for the hydrocarbon-based chains to be pendent chains or chains at the end of the hydrophilic block. In particular, it is possible for one or more pendent chains to be envisaged. In addition, the polymer may include a hydrocarbon-based chain at one end or at both ends of a hydrophilic block.

The polyurethane polyethers may be multiblock, in particular in triblock form. The hydrophobic blocks may be at each end of the chain (for example: triblock copolymer bearing a hydrophilic central block) or distributed both at the ends and in the chain (for example, multiblock copolymer). These same polymers may also be graft polymers or star polymers.

The fatty-chain nonionic polyurethane polyethers may be triblock copolymers, the hydrophilic block of which is a polyoxyethylenated chain including from 50 to 1000 oxyethylene groups. The nonionic polyurethane polyethers comprise a urethane bond between the hydrophilic blocks, giving rise to their name.

By extension, also included among the fatty-chain nonionic polyurethane polyethers are those in which the hydrophilic blocks are linked to the lipophilic blocks via other chemical bonds.

As examples of fatty-chain nonionic polyurethane polyethers that may be used in the invention, use may also be made of Rheolate 205® containing a urea function, sold by the company Rheox, or Rheolate® 208, 204 or 212, and also Acrysol RM 184®. Mention may also be made of the product Elfacos T210® containing a C12-C14 alkyl chain, and the product Elfacos T212® containing a C18 alkyl chain, from Akzo. Use may also be made of the product DW 1206B from Rohm & Haas containing a C20 alkyl chain and containing a urethane bond, provided at a solids content of 20% in water.

Use may also be made of solutions or dispersions of these polymers, particularly in water or in an aqueous/alcoholic medium. Mention may be made, as examples of such polymers, of Rheolate® 255, Rheolate® 278 and Rheolate® 244, sold by the company Rheox. Use may also be made of the products DW 1206F and DW 1206J sold by the company Rohm & Haas.

The polyurethane polyethers that may be used according to the invention are in particular those described in the article by G. Fonnum, J. Bakke and Fk. Hansen—Colloid Polym. Sci. 271, 380.389 (1993).

Even more particularly, preference is given to using a polyurethane polyether that may be obtained by polycondensation of at least three compounds comprising
(i) at least one polyethylene glycol comprising from 150 to 180 mol of ethylene oxide,
(ii) stearyl alcohol or decyl alcohol and
(iii) at least one diisocyanate.

Such polyether polyurethanes are sold in particular by the company Rohm & Haas under the names Aculyn 46® and Aculyn 44® [Aculyn 46® is a polycondensate of polyethylene glycol having 150 or 180 mol of ethylene oxide, of stearyl alcohol and of methylenebis(4-cyclohexyl isocyanate) (SMDI), at 15% by weight in a matrix of maltodextrin (4%) and water (81%); Aculyn 44® is a polycondensate of polyethylene glycol having 150 or 180 mol of ethylene oxide, of decyl alcohol and of methylenebis(4-cyclohexyl isocyanate) (SMDI), at 35% by weight in a mixture of propylene glycol (39%) and water (26%)].

Preferably, the composition according to the invention comprises one or more nonionic associative polymers, preferentially chosen from polyurethane polyethers, and/or one or more cationic associative polymers, preferably chosen from quaternized cellulose derivatives, and in particular celluloses, including quaternized hydroxyethylcelluloses modified with groups including at least one fatty chain, such as linear or branched alkyl groups, linear or branched arylalkyl groups, linear or branched alkylaryl groups including at least 8 carbon atoms, or mixtures thereof, and/or quaternized celluloses corresponding to formula (Ib) above, particularly Polyquaternium-67; preferentially one or more cationic associative polymers chosen from quaternized cellulose derivatives.

Preferably, the associative polymer(s) may be present in the composition in a total content ranging from 0.01% to 10% by weight, preferentially from 0.05% to 5% by weight, more preferentially from 0.1% to 1.5% by weight, relative to the total weight of the composition.

Preferably, the cationic associative polymer(s), chosen from quaternized cellulose derivatives, may be present in the composition in a total content ranging from 0.01% to 10% by weight, preferentially from 0.05% to 5% by weight, more preferentially from 0.1% to 1.5% by weight, relative to the total weight of the composition.

### Polyols

The composition according to the invention may also comprise at least one polyol.

Preferably, the composition according to the invention comprises one or more polyols. For the purposes of the present invention, the term “polyol” means an organic compound constituted of a hydrocarbon-based chain optionally interrupted with one or more oxygen atoms and bearing at least two free hydroxyl groups (—OH), preferably borne by different carbon atoms, it being possible for this compound to be cyclic or acyclic, linear or branched, and saturated or unsaturated.

More particularly, the polyol(s) comprise from 2 to 30 hydroxyl groups, better still from 2 to 10 hydroxyl groups, preferentially from 2 to 3 hydroxyl groups. They preferably comprise from 2 to 10 carbon atoms, particularly from 2 to 8 carbon atoms and better still from 2 to 6 carbon atoms.

Advantageously, the polyol(s) are chosen from diglycerol, glycerol, propylene glycol, propane-1,3-diol, 1,3-butylene glycol, pentane-1,2-diol, octane-1,2-diol, dipropylene glycol, hexylene glycol, ethylene glycol, polyethylene glycols, sorbitol, sugars such as glucose and mixtures thereof; preferably from glycerol, propylene glycol, propane-1,3-diol, 1,3-butylene glycol, pentane-1,2-diol, octane-1,2-diol, dipropylene glycol, hexylene glycol, ethylene glycol, sorbitol and mixtures thereof; and even better still from glycerol, propylene glycol, propane-1,3-diol, sorbitol and mixtures thereof.

Preferably, the polyol(s) may be present in the composition in a total content ranging from 0.01% to 45% by weight, better still ranging from 0.1% to 12% by weight, even better still ranging from 0.2% to 6% by weight, and preferentially ranging from 0.3% to 3% by weight, relative to the total weight of the composition.

### Thickeners

The composition according to the invention may also comprise at least one thickener.

Preferably, the composition according to the invention comprises one or more thickeners.

The thickener(s) may particularly be aqueous-phase thickeners (or hydrophilic thickeners).

The terms “thickener” or “aqueous-phase thickener” means a compound which increases the viscosity of the aqueous phase into which it is introduced at a concentration of 0.05% by weight by at least 20 cPs (20 mPa·s), preferably by at least 50 cPs (50 mPa·s), the viscosity being measured at 25° C., 1 atm, at a shear rate of 1 s⁻¹ (the viscosity can be measured using a cone/plate viscometer, a Haake R600 rheometer or the like).

The thickener is other than the surfactants, polymers, silicones, fatty substances and polyols described above. In particular, the thickener is other than the associative polymers above and than the cationic polymers above.

The thickener may advantageously be chosen from non-associative thickening polymers bearing sugar units, non-associative thickening polymers without sugar units, and mixtures thereof. Better still, the thickener may advantageously be chosen from anionic, nonionic or amphoteric non-associative thickening polymers bearing sugar units, anionic, nonionic or amphoteric non-associative thickening polymers without sugar units, and mixtures thereof.

For the purposes of the present invention, the term “sugar unit” means an oxygen-comprising hydrocarbon-based compound which has several alcohol functions, with or without aldehyde or ketone functions, and which includes at least 4 carbon atoms.

The sugar units can be optionally modified by substitution, and/or by oxidation and/or by dehydration.

The sugar units that may be included in the composition of the aqueous-phase thickening polymers of the invention are preferably derived from the following sugars: glucose, galactose, arabinose, rhamnose, mannose, xylose, fucose, anhydrogalactose, galacturonic acid, glucuronic acid, mannuronic acid, galactose sulfate, anhydrogalactose sulfate and fructose.

Mention may particularly be made, as non-associative thickening polymers bearing sugar units, of native gums, such as:
a) tree or shrub exudates, such as:
   gum arabic (branched polymer of galactose, arabinose, rhamnose and glucuronic acid);
   ghatti gum (polymer derived from arabinose, galactose, mannose, xylose and glucuronic acid);
   karaya gum (polymer derived from galacturonic acid, galactose, rhamnose and glucuronic acid);
   gum tragacanth (polymer of galacturonic acid, galactose, fucose, xylose and arabinose);
b) gums derived from algae, such as:
   agar (polymer derived from galactose and anhydrogalactose);
   alginates (polymers of mannuronic acid and of glucuronic acid);
   carrageenans and furcellerans (polymers of galactose sulfate and of anhydrogalactose sulfate);
c) gums derived from seeds or tubers, such as:
   guar gum (polymer of mannose and galactose);
   locust bean gum (polymer of mannose and galactose);
   fenugreek gum (polymer of mannose and galactose);
   tamarind gum (polymer of galactose, xylose and glucose);
   konjac gum (polymer of glucose and mannose);
d) microbial gums, such as:
   xanthan gum (polymer of glucose, mannose acetate, mannose/pyruvic acid and glucuronic acid);
   gellan gum (polymer of partially acylated glucose, rhamnose and glucuronic acid);
   scleroglucan gum (glucose polymer);
e) plant extracts, such as:
   cellulose (glucose polymer);
   starch (glucose polymer) and
   inulin.

These polymers may be physically or chemically modified.

As physical treatment, mention may be made of temperature.

As chemical treatment, mention may be made of esterification, etherification, amidation and oxidation reactions. These treatments make it possible to produce polymers that may particularly be nonionic, anionic or amphoteric.

Preferably, these chemical or physical treatments are applied to guar gums, locust bean gums, starches and celluloses.

The nonionic guar gums that may be used according to the invention may be modified with C₁-C₆ (poly)hydroxyalkyl groups.

Among the C₁-C₆ (poly)hydroxyalkyl groups, mention may be made, by way of example, of hydroxymethyl, hydroxyethyl, hydroxypropyl and hydroxybutyl groups.

These guar gums are well known from the prior art and may be prepared, for example, by reacting corresponding alkene oxides, for instance propylene oxides, with the guar gum so as to obtain a guar gum modified with hydroxypropyl groups.

The degree of hydroxyalkylation preferably ranges from 0.4 to 1.2 and corresponds to the number of alkylene oxide molecules consumed by the number of free hydroxyl functions present on the guar gum.

Such nonionic guar gums optionally modified with hydroxyalkyl groups are sold, for example, under the trade names Jaguar HP8, Jaguar HP60 and Jaguar HP120 by the company Rhodia Chimie.

The botanical origin of the starch molecules that may be used in the present invention may be cereals or tubers. Thus, the starches are chosen, for example, from corn starch, rice starch, cassava starch, barley starch, potato starch, wheat starch, sorghum starch and pea starch.

The starches may be chemically or physically modified, particularly by one or more of the following reactions: pregelatinization, oxidation, crosslinking, esterification, etherification, amidation, heat treatments.

Distarch phosphates or compounds rich in distarch phosphate will preferentially be used, for instance the product sold under the references Prejel VA-70-T AGGL (gelatinized hydroxypropyl cassava distarch phosphate), Prejel TK1 (gelatinized cassava distarch phosphate) or Prejel 200 (gelatinized acetylated cassava distarch phosphate) by the company Avebe, or Structure Zea from National Starch (gelatinized maize distarch phosphate).

According to the invention, amphoteric starches may also be used, these amphoteric starches comprising one or more anionic groups and one or more cationic groups. The anionic and cationic groups can be bonded to the same reactive site of the starch molecule or to different reactive sites; they are preferably bonded to the same reactive site.

The anionic groups may be of carboxylic, phosphate or sulfate type, preferably carboxylic type. The cationic groups may be of primary, secondary, tertiary or quaternary amine type.

The starch molecules may be derived from any plant source of starch, particularly such as corn, potato, oat, rice, tapioca, sorghum, barley or wheat. It is also possible to use hydrolyzates of the starches mentioned above. The starch is preferably derived from potato.

The non-associative thickening polymers of the invention may be cellulose-based polymers not comprising a C₁₀-C₃₀ fatty chain in their structure.

According to the invention, the term “cellulose” polymer means any polysaccharide compound having, in its structure, sequences of glucose residues joined via β-1,4 bonds; in addition to unsubstituted celluloses, the cellulose derivatives other than the polymers described above can be anionic, amphoteric or nonionic.

Thus, the cellulose-based polymers that may be used according to the invention may be chosen from unsubstituted celluloses, including those in a microcrystalline form, and cellulose ethers.

Among these cellulose-based polymers, cellulose ethers, cellulose esters and cellulose ether esters are distinguished.

Among the cellulose esters are inorganic esters of cellulose (cellulose nitrates, sulfates, phosphates, etc.), organic esters of cellulose (cellulose monoacetates, triacetates, amidopropionates, acetatebutyrates, acetatepropionates or acetatetrimellitates, for example), and mixed organic/inorganic esters of cellulose, such as cellulose acetatebutyrate sulfates and cellulose acetatepropionate sulfates. Among the cellulose ester ethers, mention may be made of hydroxypropylmethylcellulose phthalates and ethylcellulose sulfates.

Among the nonionic cellulose ethers not bearing a C₁₀-C₃₀ fatty chain, i.e. “non-associative” cellulose ethers, mention may be made of (C₁-C₄)alkylcelluloses, such as methylcelluloses and ethylcelluloses (for example, Ethocel standard 100 Premium from Dow Chemical); (poly)hydroxy(C₁-C₄)alkylcelluloses, such as hydroxymethylcelluloses, hydroxyethylcelluloses (for example, Natrosol 250 HHR provided by Aqualon) and hydroxypropylcelluloses (for example, Klucel EF from Aqualon); mixed (poly)hydroxy(C₁-C₄)alkyl(C₁-C₄)alkylcelluloses, such as hydroxypropylmethylcelluloses (for example, Methocel E4M from Dow Chemical), hydroxyethylmethylcelluloses, hydroxyethylethylcelluloses (for example, Bermocoll E 481 FQ from Akzo Nobel) and hydroxybutylmethylcelluloses.

Among the anionic cellulose ethers without a fatty chain, mention may be made of (poly)carboxy(C₁-C₄)alkylcelluloses and salts thereof. Mention may be made, by way of example, of carboxymethylcelluloses, carboxymethylmethylcelluloses (for example Blanose 7M from the company Aqualon) and carboxymethylhydroxyethylcelluloses, and the sodium salts thereof.

Among the non-associative thickening polymers not bearing sugar units that may be used according to the invention, mention may be made of acrylic acid or methacrylic acid homopolymers or copolymers, 2-acrylamido-2-methylpropanesulfonic acid homopolymers and the acrylamide copolymers thereof, alone or as mixtures, it being possible for said polymers to be crosslinked or uncrosslinked.

A first family of non-associative thickening polymers that is suitable for use is represented by acrylic acid homopolymers, preferably crosslinked.

Among the homopolymers of this type, mention may be made of those crosslinked with an allyl alcohol ether of the sugar series, for instance the products sold under the names Carbopol 980, 981, 954, 2984 and 5984 by Noveon or the products sold under the names Synthalen M and Synthalen K by 3 VSA. These polymers have the INCI name Carbomer.

The non-associative thickening polymers may also be crosslinked (meth)acrylic acid copolymers, such as the polymer sold under the name Aqua SF1 by the company Noveon.

The non-associative thickening polymers may also be chosen from crosslinked 2-acrylamido-2-methylpropanesulfonic acid homopolymers and the crosslinked acrylamide copolymers thereof.

Among the partially or totally neutralized crosslinked copolymers of 2-acrylamido-2-methylpropanesulfonic acid and of acrylamide, mention may be made in particular of the product described in Example 1 of document EP 503 853, and reference may be made to said document as regards these polymers.

Preferably, the thickener(s) may be chosen from polymers not comprising sugar units, in particular from crosslinked or uncrosslinked non-associative thickening polymers bearing acrylic or methacrylic units; in particular from acrylic or methacrylic acid homopolymers or copolymers, preferentially from crosslinked acrylic or methacrylic acid homopolymers or copolymers, alone or as mixtures; even better still from crosslinked acrylic acid homopolymers, particularly crosslinked by a pentaerythrityl allyl ether, a sucrose allyl ether or a propylene allyl ether.

Preferably, the thickener(s) may be present in the composition in a total content ranging from 0.01% to 10% by weight, better still ranging from 0.02% to 4% by weight, even better still ranging from 0.05% to 2% by weight, and preferentially ranging from 0.1% to 1% by weight, relative to the total weight of the composition.

### Other Ingredients

The composition according to the invention advantageously comprises water, which may be present in a proportion of from 65% to 98% by weight, better still from 70% to 97% by weight and preferably from 75% to 95% by weight, relative to the total weight of the composition.

The composition according to the invention may also comprise, furthermore, one or more water-miscible organic solvents other than the polyols described previously, particularly chosen from non-aromatic C1-C6 alcohols such as ethanol and/or isopropanol, aromatic alcohols such as benzyl alcohol and/or phenylethyl alcohol; and mixtures thereof. When they are present, said organic solvent(s) generally represent from 0.1% to 15% by weight and preferably from 0.5% to 10% by weight and better still from 1% to 5% by weight, of the total weight of the composition.

The composition according to the present invention may also optionally comprise one or more additives customarily used in the field, particularly chosen from antidandruff agents, anti-seborrhoea agents, vitamins and provitamins including panthenol, sunscreens, sequestrants, plasticizers, solubilizers, acidifying agents, opacifiers or pearlescent agents, antioxidants, hydroxy acids, fragrances, preserving agents, dyes and fillers.

Needless to say, those skilled in the art will take care to choose this or these additives such that the advantageous properties intrinsically associated with the composition of the invention are not, or are not substantially, adversely affected by the envisaged addition(s). The above additives may generally be present in an amount, for each of them, of between 0 and 20% by weight, relative to the total weight of the composition.

According to one embodiment, the composition further comprises:
at least one polyol, and/or
at least one associative polymer, preferably chosen from nonionic or cationic associative polymers; and/or
at least one thickener.

### Process

Another subject of the invention is a cosmetic hair treatment process, comprising a step of applying the composition as defined previously to the hair.

The cosmetic hair treatment method according to the invention may more particularly be a method for washing and/or conditioning the hair. It is preferably a method for conditioning the hair.

This method comprises a step of applying a composition according to the invention to the hair.

The composition according to the invention may be applied to wet hair or dry hair, preferably to wet hair.

The step of applying the composition according to the invention may be followed by a step of washing the hair, for example with a shampoo; this is particularly the case when the composition according to the invention is used as a pre-shampoo.

The step of applying the composition according to the invention may be preceded by a step of washing the hair, for example with a shampoo; this is particularly the case when the composition according to the invention is used as a conditioner or hair mask.

The step of applying the composition according to the invention may be followed by a leave-on time of the composition that may range from 1 to 15 minutes, preferably from 2 to 10 minutes.

The step of applying the composition according to the invention may be followed by a step of rinsing the composition, for example with water.

The step of applying the composition according to the invention may be followed, after a possible leave-on time and/or an optional rinsing step, with a step of drying, for example using a hairdryer. The hair may also be left to dry, particularly left to dry naturally.

### Methods for Determining the Amine Number, the Weight-Average Molecular Mass and the Viscosity

### A/ Amine Number

The amine number (or amine content) may be determined as described thereafter, based on a ASTM standard for example “Determination of Amine content (tertiary amine) of Amino alkyl silanes and siloxane fluids”.

The amine number may be determined by acid-base titration and defined as the amount (in milliequivalents or meq) of perchloric acid needed to neutralize 1 g of amine in the sample.

### Procedure:

weigh 10 g of sample into a 500 ml Erlenmeyer flask
add 50 ml of toluene, mix until the sample has dissolved, then add 125 ml of acetic acid
while stirring, add 5 drops of crystal violet indicator (at 0.5% by weight/volume in the acetic acid, i.e. 0.5 g of crystal violet in 100 ml of acetic acid)
titrate with 0.1 N perchloric acid HClO₄ (in the acetic acid) until the green colour disappears; note that the colour changes occur in the following order: violet→blue→green→yellow

This thus gives the amount of perchloric acid needed to neutralize the 10 g of sample.

The amine number (AN) is calculated as follows:

$$Amine number ( meq / g ) = [ ( ml {HClO}_{4} ) \times ( N {HClO}_{4} ) ] / weight of sample ( g )$$

In the present case (with N=0.1 N and sample weight=10 g), this gives the following simplified equation:

$$AN ( meq / g ) = [ ( ml {HClO}_{4} ) \times 0.1 ] / 10 = ( ml {HClO}_{4} ) / 100.$$

### B/ Weight-Average Molecular Mass

The weight-average molecular mass may be determined by any known method, particularly by dynamic light scattering, gel permeation chromatography or NMR. It is generally expressed in daltons.

Preferably, the weight-average molecular masses of the aminosilicones of formula I are measured by gel permeation chromatography (GPC) at room temperature, as polystyrene equivalents. The column used are μ styragel columns. The eluent is THF and the flow rate is 1 mL/minute. 200 μL of a solution containing 0.5% by weight of silicone in THF are injected. Detection is performed by refractometry and UV-metry.

An alternative method can be used to determine the weight-average molecular masses of these aminosilicones:
1. Using nuclear magnetic resonance (NMR) spectroscopy to determine an average general structure and;
2. Calculating the number average molecular weight from the average general structure.

The average general structure can be obtained using nuclear magnetic resonance (NMR) spectroscopy on a Bruker AV 111 600 Spectrometer operating at field strength of 14.1T; ¹H's resonate at 600 MHz. A sample can be placed in a 5 mm or 10 mm NMR tube and diluted with CDCl₃ or 0.1M Cr(AcAc)₃/CDCl₃ to a final concentration of 0.05M Cr(AcAc)₃. Inverse gated decoupling pulse sequence can be used with a pulse width of 45-degrees (¹³C: delay of ≥5 s, AQ of ≥1.65 s; ²⁹Si: delay of ≥10 s, AQ of ≥1.42 s). The ¹H NMR spectrum can be acquired using a standard pulse sequence with a 30-degrees pulse width and a delay of 212 s.

### C/ Dynamic Viscosity

The dynamic viscosity can be determined according to the following method.

Measurement conditions: 25±2° C., 1 atm., relative humidity 50±5%

Brookfield viscometer: this is a precise torque meter which is operated at a discrete rotational speed. The torque measuring system, which consists of a calibrated beryllium-copper spring that connects the drive mechanism to a rotating cone, detects the resistance to rotation caused by the presence of a fluid sample between the cone and a stationary flat plate. The resistance to rotation of the cone produces torque which is proportional to the shear stress in the fluid.

Size of the sample: 500 ml in a beaker.

Spindles used, based on the viscosity range to be measured:

Spindle no. 1: for viscosities of less than 950 mPa·s

Spindle no. 2: 950-3800 mPa·s

Spindle no. 3: 3800-4750 mPa·s

Spindle no. 4: 4750-9500 mPa·s

The viscosity measurement is started with spindle no. 1; if the viscosity is outside the range for this spindle, the next spindle will be tried, and so on until an absolute viscosity measurement is obtained. The value obtained after the spindle has been rotating for two minutes is taken as the viscosity value.

The examples that follow serve to illustrate the invention without, however, being limiting in nature. The amounts are indicated therein, unless otherwise indicated, as weight percentages of active material (% AM) relative to the total weight of the composition.

In the following examples, the amino silicone according to the invention corresponds to the formula:

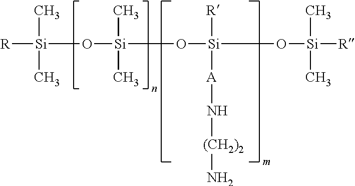

in which R═R′═R″=methyl and A=—CH₂—CH₂—CH₂—, with m=1,4-13, n=400-600 and n/m=45-300.

The amine number (AN) thereof is between 0.1 and 0.16 meq/g and the weight-average molecular mass (Mw) thereof is between 35 000 and 45 000.

### EXAMPLE 1

Composition A according to the invention and comparative composition A′ were prepared from the following ingredients (% AM):

TABLE 1

| | A | A′ |
|---|---|---|
| | (inven- | (compar- |
| Ingredients | tion) | ative) |
| BEHENTRIMONIUM CHLORIDE | 2.4 | 2.4 |
| CETEARYL ALCOHOL | 3 | 3 |
| CETYL ESTERS | 1 | 1 |
| GLYCEROL | 2 | 2 |
| AMODIMETHICONE (invention) | 0.2 | — |
| AMODIMETHICONE (AN = 0.625 meq/g and | — | 0, 2 |
| Mw = 25 000) | | |
| DIMETHICONE (XIAMETER PMX-200 | 0.1 | 0.1 |
| Silicone Fluid 60000 cst) | | |
| Preservatives | qs | qs |
| pH agent | qs pH 4.1 | qs pH 4.1 |
| Water | qs ad 100 | qs ad 100 |

Hair compositions are obtained which may be used as conditioners; the composition according to the invention is found to make it possible in particular to provide conditioning to the hair, especially a smooth feel, and coating.

On wet hair and dry hair, the sensory performance provided by composition A according to the invention is evaluated by comparison with that provided by comparative composition A′.

The evaluation is carried out on hair locks sensitized by a bleaching treatment. The locks are cleaned beforehand with a standard shampoo (DOP CAMOMILE) at a proportion of 0.3 g/g of hair and are rinsed and dried.

A shampoo is then applied to the locks (Total Repair 5 ELSEVE—L′OREAL PARIS) at a proportion of 0.3 g/g of hair and is massaged in and foamed up, and then the locks are rinsed with water for 15 seconds (flow rate=300 I/h, water temperature 35° C.).

To the washed locks, which are still damp, care composition A or A′ is applied at a proportion of 0.45 g/g of hair and is left in for 5 minutes, after which the locks are rinsed with water for 10 seconds (flow rate=300 l/h, temperature 35° C.). Evaluation is carried out on wet hair.

The locks are then dried using a hairdryer, and the evaluation is carried out on dry hair.

The following criteria are evaluated:
on wet hair: smooth feel and coating
on dry hair: smooth feel

The evaluation is performed blind, by 6 evaluators, who award a score ranging from 0 (no performance) to 5 (very good performance), in steps of 0.5, for the criterion tested.

Evaluation of the smooth feel: the evaluator takes a lock of hair at the root and slides it through the fingers over the entire length of the lock as far as the ends. The more the hair is uniform, homogeneous from the root to the end, the fewer bumps it exhibits and the less it catches on the fingers, the better the smooth feel.

Evaluation of the coating: the evaluator takes the lock of hair in the hand, starts from the root, and slides as far as the ends. The more a deposit is felt on the fibre, the better the coating.

The results obtained are as follows (average of the 6 scores):

TABLE 2

| | | A | A′ |
|---|---|---|---|
| Wet hair | Smooth feel | 4.1 ± 0.2 | 2.7 ± 0.3 |
| | Coating | 4.0 ± 0 | 2.9 ± 0.4 |
| Dry hair | Smooth feel | 4.1 ± 0.2 | 3.3 ± 0.4 |

The hair treated with the composition according to the invention is found to have significantly superior cosmetic performance by comparison with the hair treated with the comparative composition: in particular, with an improved smooth nature to the touch, both on wet hair and on dry hair, and with more present coating. The hair therefore appears to be more even, its bumps removed, and therefore repaired.

### EXAMPLE 2

Composition B according to the invention and comparative composition B′ were prepared from the following ingredients (% AM):

TABLE 3

| | B | B′ |
|---|---|---|
| | (inven- | (compar- |
| Ingredients | tion) | ative) |
| BEHENTRIMONIUM CHLORIDE | 2.4 | 2.4 |
| CETEARYL ALCOHOL | 3 | 3 |
| CETYL ESTERS | 1 | 1 |
| GLYCEROL | 2 | 2 |
| AMODIMETHICONE (invention) | 1 | — |
| AMODIMETHICONE (AN = 0.3 meq/g and | — | 1 |
| Mw = 10 000 − 20 000) | | |
| DIMETHICONE | 0.4 | 0.4 |
| (XIAMETER PMX-200 Silicone | | |
| Fluid 60000 cst) | | |
| Preservatives | qs | qs |
| pH agent | qs pH 4.1 | qs pH 4.1 |
| Water | qs ad 100 | qs ad 100 |

Hair compositions are obtained which may be used as conditioners; the composition according to the invention is found to make it possible in particular to provide the hair with conditioning, particularly a soft and smooth feel, and with regular coating, from the root to the ends.

According to a protocol identical to that of Example 1, the sensory performance provided by composition B by comparison with composition B′ is evaluated on dry hair.

The following criterion is evaluated:
on dry hair: the smooth feel

The evaluation is performed blind, by 4 evaluators, who award a score ranging from 0 (no performance) to 5 (very good performance), in steps of 0.5, for the criterion tested.

The results obtained are as follows:

TABLE 4

| | | B | B′ |
|---|---|---|---|
| Dry hair | Smooth feel | 4.0 ± 0.5 | 2.3 ± 0.6 |

The hair treated with the composition according to the invention is found to exhibit significantly superior cosmetic performance by comparison with the hair treated with the comparative composition, in particular with an improved smooth nature to the touch. The hair appears to be more uniform from the root to the end, with a softer feel result.

### EXAMPLE 3

Composition C according to the invention and comparative composition C′ were prepared from the following ingredients (% AM):

TABLE 5

| | C | C′ |
|---|---|---|
| | (inven- | (compar- |
| Ingredients | tion) | ative) |
| CETRIMONIUM CHLORIDE | 1.2 | 1.2 |
| CETEARYL ALCOHOL | 3.9 | 3.9 |
| GLYCEROL | 3 | 3 |
| AMODIMETHICONE (invention) | 0.5 | — |
| AMODIMETHICONE (AN = 0.625 meq/g and | — | 0.5 |
| Mw = 25 000) | | |
| DIMETHICONE (XIAMETER PMX-200 | 0.1 | 0.1 |
| Silicone Fluid 60000 cst) | | |
| Preservatives | qs | qs |
| Water | qs ad 100 | qs ad 100 |

Hair compositions are obtained which may be used as conditioners, for providing the hair with conditioning.

The compositions are tested on natural Caucasian hair locks, of 2.7 g and 27 cm in length.

In a first phase, the locks are cleaned using a silicone-free standard shampoo (DOP Camomile) at a proportion of 0.4 g/g of hair. The shampoo is massaged into the lock, causing it to foam up, before a leave-in time of 15 seconds, followed by a step of rinsing with water for 10 seconds (flow rate: 300 I/h, temperature: 35° C.).

In a second phase, the composition C or C′ to be tested is applied to wet hair at a proportion of 0.4 g/g of hair, before a leave-in time of 2 minutes, followed by rinsing with water for 15 seconds (flow rate: 300 I/h, temperature: 35° C.).

Lastly, the locks are dried for 30 minutes in an oven at 60° C.

The cycle of application of standard shampoo+composition C or C′+oven drying is repeated 4 more times.

At the end of the 5th cycle, the deposition of silicone on the hair is quantified by X-ray fluorescence spectrometry, via measurement of the mass concentration of elemental silicon on the hair fibre.

The amount of silicon naturally present in the untreated hair is subtracted from the measurement (i.e. deposition of silicon=amount measured on treated hair−amount measured on untreated hair).

The results obtained are as follows:

TABLE 6

| | C | C′ |
|---|---|---|
| Deposition of silicon on lock (μg/g) | 385 | 223 |
| Standard deviation | 30 | 2 |

The composition according to the invention is found to enable significantly greater deposition of silicone on the hair than that obtained with a comparative composition comprising a different amino silicone.

### EXAMPLE 4

Composition D according to the invention and comparative composition D′ were prepared from the following ingredients (% AM):

TABLE 7

| | D | D′ |
|---|---|---|
| | (inven- | (compar- |
| Ingredients | tion) | ative) |
| BEHENTRIMONIUM CHLORIDE | 3.2 | 3.2 |
| CETEARYL ALCOHOL | 7 | 7 |
| CETYL ESTERS | 1.5 | 1.5 |
| GLYCEROL | 3 | 3 |
| AMODIMETHICONE (invention) | 0.2 | — |
| AMODIMETHICONE (AN = 0.3 meq/g and | — | 0.2 |
| Mw = 10 000 − 20 000) | | |
| DIMETHICONE (XIAMETER PMX-200 | 0.1 | 0.1 |
| Silicone Fluid 60000 cst) | | |
| Preservatives | qs | qs |
| Water | qs ad 100 | qs ad 100 |

Hair compositions are obtained which may be used as hair masks; the composition according to the invention is found to make it possible in particular to provide the hair with a smooth feel and with coating.

On wet hair and dry hair, the sensory performance provided by composition D according to the invention is evaluated by comparison with that provided by comparative composition D′.

The evaluation is carried out on hair locks sensitized by a bleaching treatment and cleaned beforehand with a standard shampoo (DOP CAMOMILE), and rinsed and dried, similarly to Example 1.

A shampoo is then applied to the locks (Total Repair 5 ELSEVE—L′OREAL PARIS) at a proportion of 0.3 g/g of hair and is massaged in and foamed up, and then the locks are rinsed with water for 15 seconds (flow rate=300 I/h, water temperature 35° C.).

To the washed locks, which are still damp, care composition D or D′ is applied at a proportion of 0.45 g/g of hair and is left in for 5 minutes, after which the locks are rinsed with water for 10 seconds (flow rate=300 I/h, temperature 35° C.). Evaluation is carried out on wet hair.

The locks are then dried using a hairdryer, and the evaluation is carried out on dry hair.

The following criteria are evaluated:
on wet hair: coating
on dry hair: smooth feel and coating

The evaluation is performed blind, by 6 evaluators, who award a score ranging from 0 (no performance) to 5 (very good performance), in steps of 0.5, for the criterion tested.

The results obtained are as follows:

TABLE 8

| | | D | D′ |
|---|---|---|---|
| Wet hair | Coating | 4.0 ± 0 | 3.5 ± 0.3 |
| Dry hair | Smooth feel | 4.0 ± 0 | 3.0 ± 0.5 |
| | Coating | 4.0 ± 0 | 3.2 ± 0.4 |

The hair treated with the composition according to the invention is found to exhibit significantly superior cosmetic performance by comparison with the hair treated with the comparative composition, especially superior cosmetic repair benefits. The wet hair is more coated. After drying, moreover, the hair is treated with an improved level of smoothing and hairs that are more coated.

### EXAMPLE 5

Composition E according to the invention and comparative composition E′ were prepared from the following ingredients (% AM):

TABLE 9

| | E | E′ |
|---|---|---|
| | (inven- | (compar- |
| Ingredients | tion) | ative) |
| BEHENTRIMONIUM CHLORIDE | 3.2 | 3.2 |
| CETEARYL ALCOHOL | 7 | 7 |
| PEG-150/DECYL ALCOHOL/SMDI | 1.5 | 1.5 |
| COPOLYMER | | |
| GLYCEROL | 3 | 3 |
| AMODIMETHICONE (invention) | 1 | — |
| AMODIMETHICONE (AN = 0.625 meq/g and | — | 1 |
| Mw = 25 000) | | |
| DIMETHICONE (XIAMETER PMX-200 | 0.4 | 0.4 |
| Silicone Fluid 60000 cst) | | |
| Preservatives | qs | qs |
| Water | qs ad 100 | qs ad 100 |

Hair compositions are obtained which may be used as hair masks; the composition according to the invention is found to make it possible in particular to provide the hair with a smooth feel and with coating.

On wet hair and dry hair, the sensory performance provided by composition E according to the invention is evaluated by comparison with that provided by comparative composition E′.

The following criteria are evaluated:
on wet hair: smooth feel
on dry hair: coating

The evaluation is performed blind, by 4 evaluators, who award a score ranging from 0 (no performance) to 5 (very good performance), in steps of 0.5, for the criterion tested.

The results obtained are as follows:

TABLE 10

| | | E | E′ |
|---|---|---|---|
| Wet hair | Smooth feel | 4.75 ± 0.30 | 3.75 ± 0.65 |
| Dry hair | Coating | 4.5 ± 0.25 | 3.7 ± 0.4 |

The hair treated with the composition according to the invention is found to exhibit superior cosmetic performance in terms of smooth feel and coating by comparison with the hair treated with the comparative composition.

1. Cosmetic hair care composition, comprising:
at least one amino silicone of formula (I): 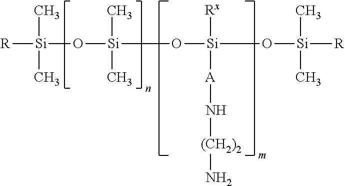
in which:
R, which is identical or different, represents a methyl group, a hydroxyl radical, or an alkoxy group comprising 1 to 4 carbon atoms;
R′ represents a methyl group or a hydroxyl radical;
A is a linear or branched divalent alkylene group comprising 3 or 4 carbon atoms;
the amino silicone having an amine number ranging from 0.1 to 0.29 meq/g and a weight-average molecular mass (Mw) ranging from 10 000 to 100 000; and
at least one non-silicone fatty substance.

2. Composition according to claim 1, in which, in the amino silicone(s) of formula (I):
R, which is identical or different, represents a methyl group and/or a hydroxyl radical; better still, the two radicals R are identical and represent a methyl group or a hydroxyl radical, even better still a methyl radical; and/or
R′ represents a methyl group; and/or
A is a linear divalent alkylene group comprising 3 or 4 carbon atoms; better still, a linear group comprising 3 carbon atoms (—CH₂CH₂CH₂—).

3. Composition according to claim 1, in which: the amino silicone(s) of formula (I) has (have) an amine number ranging from 0.1 to 0.25 meq/g, better still ranging from 0.1 to 0.19 meq/g, even better still ranging from 0.1 to 0.16 meq/g.

4. Composition according to claim 1, in which the amino silicone(s) of formula (I) has (have) a weight-average molecular mass (Mw) ranging from 20 000 to 70 000, better still from 25 000 to 50 000, even better still from 35 000 to 45000.

5. Composition according to claim 1, in which the amino silicone is of formula (I), in which m is between 1.4 and 13, better still between 1.4 and 9, even better still between 1.5 and 5; and the n/m ratio is between 45 and 330, better still between 100 and 300; and n is advantageously between 400 and 600.

6. Composition according to claim 1, in which the amino silicone is of formula (I), in which
R, which are identical, and R′, represent a methyl group,
A is a linear or branched divalent alkylene group comprising 3 or 4 carbon atoms; preferably a linear divalent alkylene group comprising 3 carbon atoms (—CH₂CH₂CH₂—),
the amino silicone has an amine number ranging from 0.1 to 0.19 meq/g and a weight-average molecular mass (Mw) ranging from 25 000 to 50 000;
preferably, m is between 1.4 and 13, the n/m ratio is between 45 and 300, and n can be between 400 and 600.

7. Composition according to claim 1, in which the amino silicone of formula (I) has a dynamic viscosity, measured at 25° C., 1 atm, ranging from 2 to 8 Pa·s (2000-8000 cps), better still from 3 to 6 Pa·s (3000-6000 cps), even better still from 3 to 5 Pa·s.

8. Composition according to claim 1, comprising the amino silicone(s) of formula (I) in a total content ranging from 0.001% to 10% by weight, in particular from 0.002% to 5% by weight, better still from 0.005% to 3% by weight, even better still from 0.01% to 2.5% by weight, preferentially from 0.02% to 2% by weight, better still from 0.05% to 1.5% by weight and even better still from 0.1% to 1.2% by weight, relative to the total weight of the composition.

9. Composition according to claim 1, in which the non-silicone fatty substance(s) are chosen from triglyceride oils of plant or synthetic origin, liquid fatty acid and/or fatty alcohol esters other than triglycerides, liquid C₆-C₁₈ hydrocarbons, solid fatty alcohols, liquid fatty alcohols, solid fatty acid and/or fatty alcohol esters, and mixtures thereof.

10. Composition according to claim 1, in which the non-silicone fatty substance(s) are present in a total content ranging from 0.1% to 20% by weight, better still from 1% to 18% by weight, preferentially from 2% to 15% by weight, even better still from 3% to 13% by weight relative to the total weight of the composition.

11. Composition according to claim 1, comprising at least one cationic surfactant, preferably chosen from the salts, such as the chlorides, the bromides or the methosulfates, of tetraalkylammonium, for instance the dialkyldimethylammonium or alkyltrimethylammonium salts in which the alkyl group comprises approximately from 12 to 22 carbon atoms, in particular the behenyltrimethylammonium, stearyltrimethylammonium, distearyldimethylammonium, cetyltrimethylammonium, dicetyldimethylammonium, benzyldimethylstearylammonium salts; the dipalmitoylethylhydroxyethylmethylammonium salts, such as dipalmitoylethylhydroxyethylmethylammonium methosulfate; and mixtures thereof; more preferentially from cetryltrimethylammonium chloride, behenyltrimethylammonium chloride, dipalmitoylethylhydroxyethylmethylammonium methosulfate, and mixtures thereof.

12. Composition according to claim 1, comprising at least one nonionic surfactant, preferably chosen from, alone or as a mixture, (C₆-C₂₄ alkyl)(poly)glycosides, and more particularly (C₈-C₁₈ alkyl)(poly)glycosides; and oxyethylenated sorbitan esters, in particular those derived from C₁₂-C₂₄ saturated fatty acids and comprising 4 to 20 ethylene oxide units.

13. Composition according to claim 1, comprising at least one additional silicone other than the amino silicones of formula (I), preferably chosen from, alone or as a mixture, non-volatile liquid non-amino silicones, and in particular polydialkylsiloxanes and more particularly polydimethylsiloxanes, especially polydimethylsiloxanes having trimethylsilyl end groups; and/or non-volatile liquid amino silicones, such as amodimethicones, aminopropyldimethicones and bisaminopropyldimethicones; and mixtures thereof.

14. Composition according to claim 1, further comprising:
at least one polyol, and/or
at least one associative polymer, preferably chosen from nonionic or cationic associative polymers; and/or
at least one thickener.

15. Cosmetic hair treatment process, comprising a step of applying the composition according to claim 1 to the hair; it being possible for said application step to be followed by a rinsing step and/or a step of drying the hair.
